(19) United States
(12) Patent Application Publication
(10) Pub. No.: US 2024/0158829 A1
(43) Pub. Date: May 16, 2024

(54) METHODS FOR BIOMOLECULE ANALYSIS EMPLOYING MULTI-COMPONENT DETECTION AGENT AND RELATED KITS

(71) Applicant: Encodia, Inc., San Diego, CA (US)

(72) Inventors: Mark S. CHEE, Encinitas, CA (US); Jason C. KLIMA, San Diego, CA (US)

(21) Appl. No.: 18/499,944

(22) Filed: Nov. 1, 2023

Related U.S. Application Data
(60) Provisional application No. 63/421,932, filed on Nov. 2, 2022.

(51) Int. Cl. C12Q 1/48; C12N 15/10

(52) U.S. Cl. CPC C12Q 1/485; C12N 15/1065

(57) ABSTRACT

The present disclosure relates to methods, systems, and kits for analysis of macromolecule analytes such as polypeptides by employing a set of multi-component detection agents. In some embodiments, the method is useful for identifying a partial sequence, or a terminal amino acid of a polypeptide analyte in a high-throughput manner. In some embodiments, the multi-component detection agent includes an RNAP portion A and an RNAP portion B which, when in proximity, can generate a detectable signal, such as specific RNA transcripts.

### CROSS-REFERENCE TO RELATED APPLICATION

This application claims priority to U.S. Provisional Application No. 63/421,932 filed Nov. 2, 2022, entitled “METHODS FOR BIOMOLECULE ANALYSIS EMPLOYING MULTI-COMPONENT DETECTION AGENT AND RELATED KITS,” which is herein incorporated by reference in its entirety for all purposes.

### REFERENCE TO AN ELECTRONIC SEQUENCE LISTING

The contents of the electronic sequence listing (776532004600SEQLIST.xml; Size: 83,855 bytes; and Date of Creation: Oct. 13, 2023) is herein incorporated by reference in its entirety.

### FIELD

This disclosure generally relates to biotechnology, and in some aspects, to highly parallel, high-throughput methods and systems for analysis of macromolecules, including peptides, polypeptides and proteins, employing multi-component detection agents, such as split such as split (bipartite) ribonucleic acid polymerase (RNAP) enzymes. In some embodiments, the methods are useful for identifying a partial sequence, or a terminal amino acid of polypeptide analytes. The disclosure finds utility in a variety of methods and related kits for high-throughput macromolecule characterization and/or identification with applications in various fields, e.g., biology and medicine.

### BACKGROUND

Proteomics is the study of the structure and function of proteins in biological systems and encompasses a wide range of applications, including protein expression profiling in healthy versus diseased states of an organism, analyzing the interaction of proteins in living organisms, and mapping of protein modifications and identification of how, when and where proteins are modified within a living cell. Despite significant advances, there remains a need in the art for improved techniques for the identification and quantification of proteins in biological samples. For example, although high-throughput techniques have been developed for sequencing and/or analyzing DNA and RNA within a biological sample, such advances are still needed at the protein level.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

Traditionally, mass spectrometry (MS) has been employed for proteomic characterization. However, MS suffers from a number of drawbacks, including the requirement for relatively large sample sizes and limitations associated with quantification and dynamic range. For example, since proteins ionize at different levels of efficiencies, relative amounts are difficult to compare between samples. More recently, advances have been made in the field of digital analysis of proteins by end sequencing (referred to as DAPES) as disclosed, for example, by Mitra and Tessler in PCT Publication No. WO2010/065531. In this method, surface bound peptides are directly sequenced using a modified Edman degradation step followed by detection, such as with a labeled antibody. More specifically, the N-terminal amino acid of an immobilized protein is first reacted with phenylisothiocyanate (PITC) to form a phenylthiocarbamoyl derivative (PTC-derivative). A labeled antibody which binds both the phenyl group of the PTC-derivative and the side chain of the N-terminal amino acid is then used for detection. After detection of the bound antibody, the antibody is stripped and the procedure repeated with antibodies that will detect other PTC-derivatives (i.e., other N-terminal amino acids). By repeating the above binding, detection and stripping steps using 20 unique antibodies that recognize each of the 20 PTC-derivatives (one for each of the 20 naturally occurring amino acids), the identity of all the N-terminal amino acids of the immobilized protein can be determined. The terminal amino acids of the immobilized proteins are then removed, and the procedure repeated for the newly exposed N-terminal amino acids.

A modification of DAPES was disclosed by Havranek and Borgo in U.S. patent U.S. Ser. No. 10/852,305 B2. In this method, single molecule sequencing of peptides is achieved by contacting the peptide with one or more fluorescently labelled N-terminal amino acid binding proteins (NAABs), detecting the fluorescence of a NAAB bound to the N-terminal amino acid, identifying the N-terminal amino acid based on the fluorescence detected, removing the NAAB from the peptide, and repeating with NAABs that bind to different N-terminal amino acids. Following such steps, the N-terminal amino acid is cleaved from the polypeptide by Edman degradation, and the procedure repeated for the newly-exposed N-terminal amino acids.

Other techniques for characterizing proteins include those disclosed in U.S. Patent Application Publications US2003/0138831, US2014/0349860, and US2015/0087526 and in PCT Application Publications WO2010/065322 and WO2013/112745, each of which is incorporated by reference in its entirety for all purposes.

However, current reagents and techniques are somewhat limited particularly in the context of detection of a single molecule immobilized on a solid support, including low signal-to-noise ratios, lacking the ability to control the binding reaction, as well as non-specific binding to the support (e.g., high background fluorescence). Despite the advances that have been made in this field, there remains a significant need for improved techniques relating to polypeptide sequencing and/or analysis, as well as to methods, systems, and kits for accomplishing the same. The present disclosure fulfills these and other needs, as evident in reference to the following disclosure.

These and other aspects of the invention will be apparent upon reference to the following detailed description. To this end, various references are set forth herein which describe in more detail certain background information, procedures, compounds and/or compositions, and are each hereby incorporated by reference in their entireties.

In some embodiments, provided is a method for analyzing a macromolecule analyte, comprising the steps of:
a) contacting the macromolecule analyte with a first plurality of binding agents,
wherein the macromolecule analyte is attached to a solid support and associated with (i) an RNA polymerase (RNAP) portion A, and (ii) a recording tag comprising a plurality of promoters,
wherein each binding agent comprises (i) an RNAP portion B, and (ii) a binding moiety, and wherein upon binding between a first motif of the macromolecule analyte and the binding moiety of a first binding agent of the first plurality of binding agents, the RNAP portion A associated with the macromolecule analyte and the RNAP portion B of the first binding agent are brought into proximity to associate and form a first functional RNAP enzyme, which initiates transcription from a corresponding first promoter of the plurality of promoters to generate a first RNA transcript;
b) collecting the first RNA transcript, and removing the first functional RNAP enzyme or a portion thereof from the recording tag associated with the macromolecule analyte;
c) contacting the macromolecule analyte with a second plurality of binding agents,
wherein upon binding between a second motif of the macromolecule analyte and the binding moiety of a second binding agent of the second plurality of binding agents, the RNAP portion A associated with the macromolecule analyte and the RNAP portion B of the second binding agent are brought into proximity to associate and form a second functional RNAP enzyme, which initiates transcription from a corresponding second promoter of the plurality of promoters to generate a second RNA transcript, and
d) analyzing the first and second RNA transcripts, thereby analyzing the first and second motifs of the macromolecule analyte.

In some embodiments, the first plurality of binding agents is the same or is essentially the same as the second plurality of binding agents. The first plurality of binding agents is essentially the same as the second plurality of binding agents when, for example, the plurality of binding agents is re-used at the next binding round(s). In some embodiments, during use in the first binding round, the plurality of binding agents may become partially depleted (if some binding agents are irreversibly bound to analytes or solid support), but can still be used in the second or higher order binding rounds.

In some embodiments, collecting the first RNA transcript, removing the first binding agent from the macromolecule analyte, and removing the first functional RNAP enzyme from the recording tag can be performed by one or more washing steps. For instance, the first RNA transcript and the first binding agent (comprising the RNAP portion B, such as RNAP_{C}) can be separated from the solid support and the macromolecule analyte and the recording tag associated therewith, whereas optionally the RNAP portion A (such as RNAPN) can remain associated with the macromolecule analyte and/or the recording tag. In some embodiments, the first RNA transcript can be collected by separating it from the first binding agent, for instance, by using a capture probe (e.g., comprising poly(dT)) to capture RNA transcripts having polyA tails.

In some embodiments, removing of the first or higher order (e.g., second, third, and so on) functional RNAP enzyme or a portion thereof from the recording tag associated with the macromolecule analyte is performed by disrupting the first or higher order functional RNAP formed by RNAP portion A and RNAP portion B brought into proximity. In other embodiments, removing of the first or higher order functional RNAP enzyme or a portion thereof from the recording tag associated with the macromolecule analyte is performed by disrupting the association of RNAP portion A with the solid support, while the first or higher order functional RNAP enzyme may or may not remain intact. In some embodiments, the first or higher order functional RNAP enzyme is removed from the system by washing away from the solid support. In some embodiments, the removing step comprise removing the first or higher order binding agent from the macromolecule analyte and the first or higher order functional RNAP enzyme from the recording tag associated with the macromolecule analyte.

Steps of collecting the first or higher order RNA transcript and removing the first or higher order functional RNAP enzyme or a portion thereof from the recording tag can be performed in any order, either sequentially or simultaneously (i.e., as a single step). In preferred embodiments, RNA transcripts are collected before removing the first or higher order functional RNAP enzyme or a portion thereof from the recording tag. Collection of RNA transcripts can be performed in a bulk manner (i.e., by separating a liquid fraction from the fraction that comprises solid support(s) with associated macromolecules), or in a specific manner (e.g., by employing a nucleic acid hybridization to bind a capture RNA transcripts). The latter can be performed by using capture probes (e.g., comprising poly(dT) or other sequences complementary to a specific region within RNA transcripts) to capture RNA transcripts.

In some embodiments, a cycle-specific barcode can be attached to the first RNA transcript prior to or after it is collected.

In some embodiments, provided is also a method for analyzing a macromolecule analyte, comprising the steps of:
a) providing the macromolecule analyte attached to a solid support, wherein the macromolecule analyte is associated with (i) an RNAP portion A, and (ii) a recording tag comprising a plurality of promoters, and optionally comprising an analyte-specific barcode;
b) contacting the macromolecule analyte with a plurality of binding agents capable of binding to the macromolecule analyte, wherein each binding agent of the plurality of binding agents is joined to an RNAP portion B, whereby binding between the macromolecule analyte and a first binding agent of the plurality of binding agents brings the RNAP portion A and an RNAP portion B joined to the first binding agent into sufficient proximity to interact with each other and form a first functional RNAP enzyme configured to initiate transcription from a first promoter of the plurality of promoters;
c) following binding of the first binding agent to the macromolecule analyte, generating a first RNA transcript by the first functional RNAP enzyme from the recording tag associated with the macromolecule analyte;
d) collecting the first RNA transcript, disrupting the first functional RNAP enzyme or washing the first functional RNAP enzyme away from the recording tag associated with the macromolecule analyte;
e) optionally, removing a portion of the macromolecule analyte;
f) repeating steps (b), (c), (d) and, optionally, (e) sequentially one or more times by replacing the first binding agent with a second or higher order binding agent capable of binding to the macromolecule analyte, wherein binding between the macromolecule analyte and a second or higher order binding agent of the plurality of binding agents brings the RNAP portion A and an RNAP portion B joined to the second or higher order binding agent into sufficient proximity to interact with each other and form a second or higher order functional RNAP enzyme configured to initiate transcription from a second or higher order promoter of the plurality of promoters, thereby generating and collecting a second and, optionally, higher order RNA transcript;
g) analyzing the first, the second and, optionally, the higher order RNA transcripts, which comprises identifying RNA transcript lengths and/or at least partial sequences of the first, the second and, optionally, the higher order RNA transcripts, thereby analyzing the macromolecule analyte.

In some embodiments, also provided herein is a system for analyzing a plurality of macromolecule analytes, comprising:
a) the plurality of macromolecule analytes attached to a solid support, wherein each macromolecule analyte from the plurality of macromolecule analytes is associated with (i) an RNA polymerase (RNAP) portion A, and (ii) a recording tag comprising a plurality of promoters comprising at least a first promoter and a second promoter;
b) a plurality of binding agents capable of binding to a macromolecule analyte of the plurality of macromolecule analytes and comprising at least a first binding agent and a second binding agent, wherein each binding agent of the plurality of binding agents comprises (i) an RNAP portion B, and (ii) a binding moiety; wherein upon binding between a first motif of the macromolecule analyte and the binding moiety of the first binding agent the RNAP portion A associated with the macromolecule analyte and the RNAP portion B of the first binding agent are brought into proximity to form a first functional RNAP, which initiates transcription from the first promoter to generate a first RNA transcript; and wherein
upon binding between a second motif of the macromolecule analyte and the binding moiety of the second binding agent, the RNAP portion A associated with the macromolecule analyte and the RNAP portion B of the second binding agent are brought into proximity to form a second functional RNAP, which initiates transcription from the second promoter to generate a second RNA transcript

In some embodiments, also provided herein is a system for analyzing a plurality of macromolecule analytes, comprising:
a) the plurality of macromolecule analytes attached to a solid support, wherein each macromolecule analyte from the plurality of macromolecule analytes is associated with (i) an RNAP portion A, and (ii) a recording tag comprising a plurality of promoters, and optionally comprising an analyte-specific barcode;
b) a plurality of binding agents capable of binding to the macromolecule analyte, wherein each binding agent of the plurality of binding agents is joined to an RNAP portion B, whereby binding between the macromolecule analyte and a first binding agent of the plurality of binding agents brings the RNAP portion A and an RNAP portion B joined to the first binding agent into sufficient proximity to interact with each other and form a first functional RNAP enzyme configured to initiate transcription from a first promoter of the plurality of promoters.

### BRIEF DESCRIPTION OF THE DRAWINGS

Non-limiting embodiments of the present invention will be described by way of example with reference to the accompanying figures, which are schematic and are not intended to be drawn to scale. For purposes of illustration, not every component is labeled in every figure, nor is every component of each embodiment of the invention shown where illustration is not necessary to allow those of ordinary skill in the art to understand the invention.

FIGS. 1A-1D. FIG. 1A illustrates an exemplary configuration of the disclosed system. Four different binding agents joined to a different RNAP portion B (e.g., a C-terminal portion of a specific RNAP enzyme, indicated as RNAPC) are allowed to contact an immobilized macromolecule analyte attached to a support (e.g., a bead or a plate), wherein the macromolecule analyte is associated with (i) an RNAP portion A (e.g., an N-terminal portion of an RNAP enzyme, indicated as RNAPN, which can form a functional RNAP when associating with any one of the indicated RNAPC fragments), and (ii) a recording tag comprising a plurality of promoters. FIG. 1B. When the binding moiety of one of the binding agents binds to the immobilized macromolecule analyte, the RNAP_{N} and RNAP_{C} are brought into proximity and bind each other, resulting in association and formation of a functional RNAP enzyme. In this example, RNAPN is configured to associate with each of four different RNAP_{C} (e.g., T3-RNAP_{C} for promoter P_{T3}, T7-RNAP_{C} for promoter P_{T7}, CTGA-RNAP_{C} for promoter PCTGA, and K1F-RNAP_{C} for promoter NO, thereby forming functional RNAP enzymes for promoter-specific transcription from the corresponding promoter in the recording tag. FIG. 1C. RNA transcripts produced in FIG. 1B are released into solution and collected, and the functional RNAP enzyme is disrupted and/or washed away. The system is now ready for repeating the steps of macromolecule analyte binding and RNA transcript generation. FIG. 1D. RNA transcripts generated by the functional RNAP enzyme from the recording tag have different lengths depending on which promoter is used by the functional RNAP enzyme.

FIG. 2 illustrates an exemplary alternative recording tag architecture. Four different DNA promoters are present in a single DNA molecule and conjugated to a single functional site on the solid support (bead), wherein one unique or up to four different analyte-specific barcodes (BC_{Peptide}) correspond to a single macromolecule, such as polypeptide, covalently linked to the recording tag or to a chemical linker fusing the bead to the recording tag. Herein and in other figures “P” refers to a promoter, and “BC” refers to a barcode. Resulting RNA transcripts are of the same length, and therefore macromolecule analyte motifs are analyzed by promoter-specific barcodes instead of RNA transcript lengths.

FIG. 3 illustrates another exemplary recording tag architecture. Each of four different DNA promoters is present on a different DNA molecule, and these DNA molecules are conjugated to a single functional site on the bead, and each comprises analyte-specific barcode (BC_{Peptide}) corresponding to one macromolecule covalently linked to the recording tag. Resulting RNA transcripts are of the same length, and therefore macromolecule analyte motifs are analyzed by promoter-specific barcodes instead of RNA transcript lengths.

FIG. 4 illustrates yet another exemplary alternative recording tag architecture. Four different DNA promoters are present as a single DNA-based structure, which folds into a multi-hairpin structure (e.g., DNA origami) that comprises a plurality of promoters and is conjugated to a single functional site on the bead. Four different analyte-specific barcodes (BC_{Pepticle}) correspond to one macromolecule covalently linked to the recording tag. Each promoter element and analyte-specific barcode have a downstream Poly(A) and transcription termination sequence. Resulting RNA transcripts are of the same length, and therefore macromolecule analyte motifs are analyzed by promoter-specific barcodes instead of RNA transcript lengths.

### DETAILED DESCRIPTION

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured. All publications, including patent documents, scientific articles and databases, referred to in this application are incorporated by reference in their entireties for all purposes. Citation of the publications or documents is not intended as an admission that any of them is pertinent prior art, nor does it constitute any admission as to the contents or date of these publications or documents.

The present invention provides novel methods, systems and compositions which utilize a two-component detection system, such as a bipartite split RNA polymerase (RNAP), for highly parallel; high-throughput macromolecule analysis, characterization, quantitation and/or sequencing. Split proteins have been used for the detection and/or quantification of protein interactions, such as protein-fragment complementation assays (Michnick et al., Nat Rev Drug Discov 6, 569-82 (2007); Remy & Michnick, Methods Mol Biol 1278, 467-81 (2015); U.S. Patent Application Publication No. US 2008/0248463), split protein complementation (Shekhawat & Ghosh, Curr Opin Chem Biol 15, 789-97 (2011)), or bimolecular fluorescence complementation (Miller et al., J Mol Biol 427, 2039-55 (2015); Kerppola, T. K., Chem Soc Rev 38, 2876-2886 (2009)). Split RNAP enzymes in particular have also been used as a biosensor platform to control several parallel biological circuits, to study protein-protein interactions or to express within a cell a guide-RNA for an RNA-guided nuclease system (Pu J, et al., 2017; Pu J, et al., J Am Chem Soc. 2017; Temme K, et al., 2012; Meyer A J, et al., 2015; U.S. Patent Application Publications No. US2020/0332371, US2015/0368625 and US2020/0199599 incorporated herein by reference).

In the methods disclosed herein, split RNAP enzymes are used to analyze a plurality of macromolecule analytes immobilized on a solid support by utilizing a plurality of binding agents capable of binding to at least portions of macromolecule analytes, wherein each binding agent of the plurality of binding agents is joined to an RNAP portion B (generating binding agent-RNAP portion B conjugates). An RNAP portion B can be any portion of a functional RNAP, for instance, a C-terminal portion of an RNAP (RNAP_{C}), such as T3-RNAP_{C} for promoter P_{T3}, T7-RNAP_{C} for promoter PT7, CTGA-RNAP_{C} for promoter P_{CTGA}, and K1F-RNAP_{C} for promoter P_{KIF} shown in the figures. Each analyzed macromolecule analyte is associated with (i) an RNAP portion A, and (ii) a recording tag comprising a plurality of promoters. An RNAP portion A can be any portion of a functional RNAP that can bind an RNAP portion B in order to form the functional RNAP. For instance, an RNAP portion A can be an N-terminal portion of an RNAP (RNAP_{N}), such as the RNAPN shown in the figures. Binding between a macromolecule analyte and a first binding agent of the plurality of binding agents brings the RNAP portion A and an RNAP portion B joined to the first binding agent into sufficient proximity to interact with each other and form a first functional RNAP enzyme configured to initiate transcription from a first promoter of the plurality of promoters. Thus, following binding of the first binding agent to the macromolecule analyte, a first RNA transcript is generated by the first functional RNAP enzyme from the recording tag associated with the macromolecule analyte; then, the first RNA transcript is collected, and the first functional RNAP enzyme is disrupted, inactivated, blocked from binding to the recording tag, and/or removed from the recording tag, from the macromolecule analyte, and from the solid support (e.g., by washing away the first functional RNAP enzyme or a portion thereof). In some embodiments, the first functional RNAP enzyme is disrupted by inactivating, blocking, and/or removing (e.g., by washing away) the first binding agent (which comprises the RNAP portion B), whereas the RNA portion A remains associated with the macromolecule analyte and/or the recording tag but is rendered unable to catalyze transcription until another binding agent comprising an RNAP portion B is brought into proximity and bind with the RNA portion A in a subsequent cycle. In some embodiments, the first functional RNAP enzyme is disrupted by dissociating the RNAP portion B from the binding moiety in the first binding agent, such that the dissociated RNA portion B can be removed, whereas the binding moiety can remain bound to the macromolecule analyte. Optionally, the first motif bound by the binding moiety of the first binding agent can be modified prior to and/or after binding of the first binding agent. Optionally, prior to a subsequent cycle, the first motif bound by the binding moiety of the first binding agent can be cleaved from the macromolecule analyte, e.g., to expose a motif due to the cleavage. In the subsequent cycle, the binding moiety of a second binding agent can bind to a second motif without cleaving the first motif, or alternatively, the binding moiety of the second binding agent can bind to a second motif which is exposed or generated after cleaving the first motif. The second motif can optionally be modified prior to binding to the binding moiety of a second binding agent.

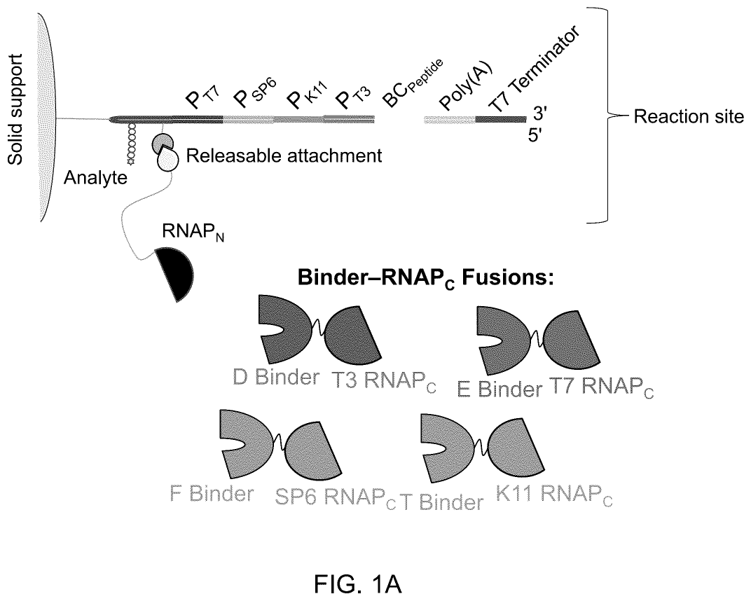

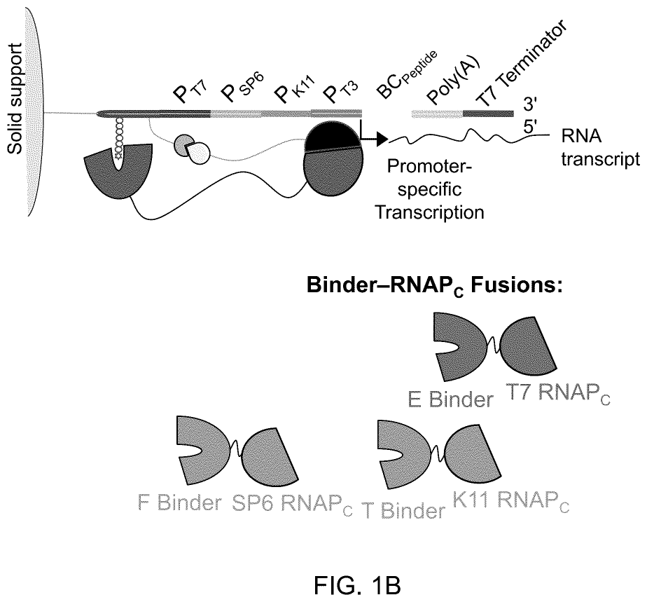

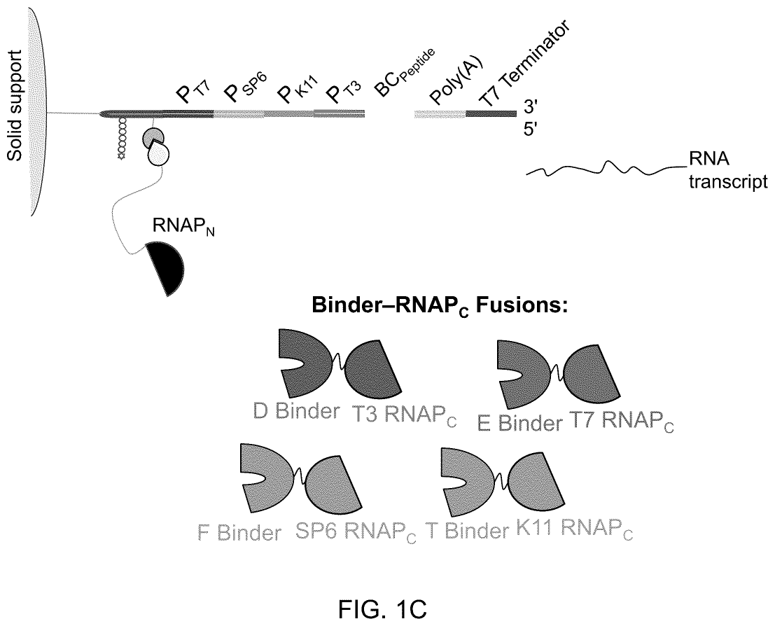

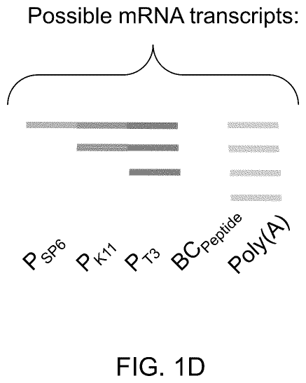

These steps can be repeated sequentially one or more times by replacing the first binding agent with a second or higher order binding agent capable of binding to the macromolecule analyte, wherein binding between the macromolecule analyte and a second or higher order binding agent brings the RNAP portion A and an RNAP portion B joined to the second or higher order binding agent into sufficient proximity to interact with each other and form a second or higher order functional RNAP enzyme configured to initiate transcription from a second or higher order promoter of the plurality of promoters, thereby generating and collecting a second and, optionally, higher order RNA transcript. The second or higher order promoter can be a promoter that is different from the first promoter. By way of example, in FIG. 1B, the first promoter is P_{T3} (bound by RNAP_{N}-T3-RNAP_{C}) and the second or higher order promoter can be promoter P_{T7}, promoter P_{CTGA}, or promoter P_{K1F}, depending on which binding moiety-RNAP_{C} fusion (the second or higher order binding agent) is brought into proximity with RNAP_{N} by the motif in the macromolecule analyte.

In preferred embodiments, the disclosed methods allow for at least partial identification of a component of each of the analyzed immobilized macromolecules based on information obtained from analyzed RNA transcripts. Identifying information regarding binding agents that were bound to the immobilized macromolecules at each binding cycle are incorporated into RNA transcripts produced at each cycle and can be decoded during the analysis of RNA transcript sequences and/or lengths.

The disclosed methods of macromolecule analysis offer significant advantages over methods known in the art. First, they offer very high multiplexity during analysis. In preferred embodiments, immobilization of macromolecule analysis from samples occurs in unbiased manner, and sometimes with preference to lower abundant macromolecules. All steps of the methods can be performed on multiple samples simultaneously. Sample identities can be barcoded in the recording tags associated with immobilized macromolecule analytes extracted from a particular sample, and thus, multiple samples can be combined together in a single assay. Further, binding agents capable of binding to macromolecule analytes may not be analyte-specific, but rather may recognize a component shared among multiple macromolecule analytes, such as an N-terminal amino acid (NTAA) residue, a functionalized NTAA residue, a terminal dipeptide, a functionalized terminal dipeptide, and so on, including disjointed regions of a macromolecule analyte if the analyte forms secondary structures and/or tertiary structures on the solid support. Second, an RNA transcript output produced during each binding cycle provides additional advantages. Unlike optical reporters, RNAP-based reporters are not limited by spectral overlap considerations, providing opportunity for utilizing multiple orthogonal RNAP enzymes. Sequences of produced RNA transcripts can be analyzed using well-established high-throughput complementary deoxyribonucleic acid (cDNA) and ribonucleic acid (RNA) sequencing tools. In addition, RNA signal can be measured in a variety of sensitive ways, including but not limited to fluorescence detection or nanoparticle detection, and can be amplified by reverse transcription polymerase chain reaction (RT-PCR) or other methods for even more sensitive detection.

In preferred embodiments, each binding agent of the plurality of binding agents is joined to a different (or modified) RNAP portion B that has specificity towards a different promoter from the plurality of promoters located on the recording tag associated with a macromolecule analyte. Therefore, by analyzing the first, the second and, optionally, the higher order RNA transcripts collected after several sequential steps can be analyzed to derive the identity of binding agents that were bound to the macromolecule analyte during the described process, since binding between the macromolecule analyte and a particular binding agent creates a unique output—that is, generation of a particular RNA transcript. In preferred embodiments, by collecting and analyzing RNA transcripts after each binding iteration, both identity and order of all binding events (collectively, a binding history) can be recreated or derived for each macromolecule analyte, and the process can be done in parallel for multiple (hundreds, thousands, or more) different macromolecule analytes. Such binding history of macromolecule analytes can be then used to obtain important structural information regarding these multiple different macromolecule analytes.

In some embodiments, the RNAP portion A and the RNAP portion B are truncated portions of the first functional RNAP enzyme.

In some embodiments, the RNAP portion A associated with each macromolecule analyte from the plurality of macromolecule analytes is identical or substantially identical.

In preferred embodiments, the RNAP portion A (e.g., RNAPN) and a set of orthogonal RNAP portion Bs (e.g., RNAP_{C} variants) are derived from T7 RNA polymerase, or a T7-related RNA polymerase, such as a bacteriophage RNA polymerase that comprises an amino acid sequence having at least 30%, at least 40%, at least 50%, at least 60%, at least 70%, at least 80%, or at least 90% sequence identity to any one of the amino acid sequences set forth in SEQ ID NO: 1-SEQ ID NO: 4. In preferred embodiments, the RNAP portion A comprises an amino acid sequence having at least 30%, at least 40%, at least 50%, at least 60%, at least 70%, at least 80%, or at least 90% sequence identity to any one of the amino acid sequences set forth in SEQ ID NO: 5-SEQ ID NO: 9. In preferred embodiments, each of the RNAP portion Bs used in the system comprises an amino acid sequence having at least 30%, at least 40%, at least 50%, at least 60%, at least 70%, at least 80%, or at least 90% sequence identity to any one of the amino acid sequences set forth in SEQ ID NO: 10-SEQ ID NO: 43, and each of the RNAP portion Bs drive transcription from unique DNA promoters located in the plurality of promoters upon complementation of the RNAP portion A and RNAP portion B and therefore functional RNAP enzyme assembly, thereby permitting the transcription of unique output RNA signals (creating a set of orthogonal RNAP portion Bs).

In some embodiments, a T7, T3, K11 or SP6 RNA polymerase, or a T7-related RNA polymerase is separated into an RNAP portion A and an RNAP portion B by selecting an amino acid boundary, junction, or split point that separates or decouples the promoter-specificity and polymerization domains from a domain that transitions a transcription initiation complex or activity to a transcription elongation complex or activity. In some particular embodiments, the RNAP portion A and one of the RNAP portion Bs are formed by splitting the T7 RNAP protein at position 179, whereas the RNAP portion A comprises SEQ ID NO: 5 and one of the RNAP portion Bs comprises SEQ ID NO: 10. In other embodiments, the RNAP portion A and one of the RNAP portion Bs are formed by splitting the T7 RNAP protein at a position other than position 179, for example, at position 178, 180, 181, 182, or any other position as disclosed in US20150368625 A1, incorporated herein by reference. Preferably, a split site in a T7-related RNA polymerase used to produce an RNAP portion A and/or an RNAP portion B is at least partially solvent exposed and not located in the DNA-binding region of RNAP.

The provided methods permit the detection, quantitation or analysis of a plurality of macromolecule analytes simultaneously, e.g., multiplexing. A plurality of macromolecule analytes that can be analyzed simultaneously can include 100 or more different macromolecules, 500 or more different macromolecules, 1000 or more different macromolecules, 5000 or more different macromolecules, 10,000 or more different macromolecules, 100,000 or more different macromolecules, or 1,000,000 or more different macromolecules. Such high level of multiplexing can be achieved by utilizing: i) efficient immobilization of different macromolecule analytes to a solid support; ii) a limited number of binding agents (for example, 4, 5, 6, 7, 8, 9, or 10 different binding agents) that are capable of binding to hundreds or thousands of different macromolecule analytes; iii) a nucleic acid sequencing readout of the methods, which permits utilizing highly parallel next-generation sequencing (NGS) approaches; and iv) bioinformatical decoding of the binding history of macromolecule analytes based on the produced RNA transcript sequences and/or lengths, which provides at least partial or probabilistic structural information regarding the analyzed macromolecule analytes.

In preferred embodiments, binding agents suitable to provide a very high level of multiplexing in the disclosed methods comprise binding agents capable of binding to an N-terminal amino acid (NTAA) of immobilized polypeptide analytes, or capable of binding to an N-terminal amino acid (NTAA) of immobilized polypeptide analytes functionalized with a chemical moiety. In these embodiments, identifying information regarding the NTAA residues of immobilized polypeptide analytes gets encoded in structures of the produced RNA transcripts, allowing for at least partial identification of polypeptide analyte sequences. In other embodiments, binding agents capable of binding to dipeptides, tripeptides, terminal dipeptides, or terminal tripeptides located within immobilized polypeptide analytes can be used (see, e.g., affinity reagent probes disclosed in US20200318101 A1 and US20220236282 A1, incorporated herein by reference).

In some embodiments, the disclosed methods further comprise the following step: modifying (functionalizing) the NTAA of the polypeptide analyte with a chemical moiety to produce a functionalized NTAA of the polypeptide analyte. In some embodiments, binding agents each conjugated to an RNAP portion B are capable of binding to a functionalized N-terminal amino acid (NTAA) residue of the polypeptide analyte. Functionalization of the NTAA residues of polypeptide analytes provides a “handle” for binding agents, and is used to achieve a better selectivity or affinity for engineered binding agents that are configured to specifically recognize and bind one or more functionalized NTAA residues of polypeptide analytes. In these embodiments, the identity of NTAA residues of the immobilized polypeptide analytes can be decoded by analyzing RNA transcripts after binding between the immobilized polypeptide analytes and the binding agents each conjugated to an RNAP portion B.

In some embodiments, a plurality of binding agents is used in the disclosed methods, wherein each binding agent of the plurality of binding agents is capable of binding to a particular functionalized NTAA residue, such as a functionalized N-terminal Ala, a functionalized N-terminal His, a functionalized N-terminal Trp and so on. In other embodiments, the plurality of binding agents used in the disclosed methods comprises binding agents that exhibit less selective binding, where a binding agent may bind with similar affinity to two or more different functionalized NTAA residues. For example, amino acid group-specific (i.e., class-specific) binding agents may be utilized, which are capable of binding to a few structurally similar functionalized NTAA residues (such as a functionalized N-terminal Val and a functionalized N-terminal Ile). In these embodiments, the disclosed sequencing method may provide only a partial identification of a particular N-terminal residue of the polypeptide analyte (such as the particular NTAA residue may be one from the amino acid group or class, which correspond to the selectivity of the binding agent bound to the NTAA residue in the current binding cycle). When such information can be assigned for several consecutive amino acid residues of a polypeptide analyte, the identity of the polypeptide analyte can be decoded with a certain probability based on comparison of the probabilities of the consecutive amino acid residues with theoretical polypeptide sequences extracted from a genomic or proteomic database.

In some embodiments, since the amount of RNA transcript produced from a given reaction site depends on binding agent concentration as well as binding agent affinity, the binding agent concentrations in the binding agent mix (the plurality of binding agents) is formulated so that each binding agent is fixed at a concentration equal to its on-target thermodynamic dissociation constant (Kd) for the macromolecule analyte NTAA or motif. Thus, stochastic but uniform RNA production rates at each reaction site in each cycle can be assumed, independent of P1 (NTAA) identity (assuming each binding agent is capable of binding to a particular NTAA of polypeptide analytes). Then, the amount of RNA transcripts produced from a given reaction site in a given cycle is dependent on the allocated time given to the reaction. After NGS of the collected RNA transcripts, the P1 (NTAA) probability of polypeptide analytes is bioinformatically analyzed from the ratio of RNA transcript lengths (or promoter-specific barcode sequences) with a given analyte-specific barcode (polypeptide barcode) in a given cycle from NGS data. In order to get approximately uniform sequence coverage over all of the cycles, RNA transcripts from each cycle are combined in equimolar ratios prior to NGS. In preferred embodiments, the allocated reaction time is long enough such that a few RNA transcripts are produced per reaction site per cycle, which can be achieved because of uniform distribution of binding agents over the solid supports (e.g., beads) and uniform binding probabilities for all binding agents because their concentrations are fixed at their on-target Kds.

In some embodiments, the disclosed methods further comprise a NTAA or functionalized NTAA cleavage step, where the NTAA or functionalized NTAA residues of the immobilized polypeptide analytes are cleaved chemically or enzymatically, generating a newly formed NTAA or functionalized NTAA residues on the same immobilized polypeptide analytes. Then, the steps of i) functionalizing NTAA residues of polypeptide analytes with a chemical moiety, and ii) binding between the immobilized polypeptide analytes and the binding agents each conjugated to an RNAP portion B are repeated, followed by analysis of newly synthesized RNA transcripts, which provide identifying information for the newly exposed (formed) NTAA residues of polypeptide analytes. During the above-described process, at least partial or incomplete sequence information regarding the immobilized polypeptide analytes can be obtained, which can result in identification of these polypeptide analytes.

Functionalizing reagent can be chosen to increase affinity, specificity and/or selectivity of binding agents towards particular terminal amino acid residues. Examples of such functionalizing reagents and binding agents that can bind to NTAA or functionalized NTAA with certain levels of specificity and selectivity are disclosed in the following patent publications, incorporated herein by reference: U.S. Pat. No. 9,435,810 B2, WO2010/065531 A1, US 2019/0145982 A1, US 2020/0348308 A1, US 2019/0145982 A1, U.S. Pat. No. 9,435,810 B2, US 2023/0220589 A1, US 2022/0283175 A1.

In some preferred embodiments, the N-terminal functionalizing reagent that is used to modify polypeptide analytes is selected from the group consisting of compounds of the following formula:

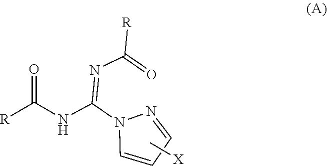

wherein R is CH₃, CF₃, OC(CH₃)₃, or OCH₂C₆H₅,
and X is H, CH₃, CF₃, CF₂H, or OCH₃;

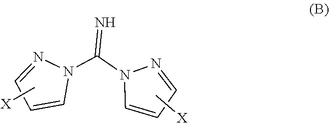

wherein X is H, CH₃, CF₃, CF₂H, OCH₃, or SO₂NH₂;

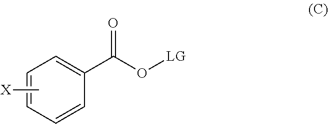

wherein X is H, F, Cl, OCH₃, OCF₃, CN, or SO₂NH₂,
and LG is succinimide, pentafluorophenyl, or tetrafluorophenyl;
and

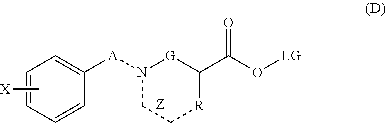

wherein X is H, F, Cl, NH₂, OCH₃, OCF₃, CN, or SO₂NH₂, A=CONH or SO₂, G=0 or 1 CH₂, R is any amino acid or unnatural amino acid, and Z ring=0 (not there), 1, 2, or 3 CH₂.

In some embodiments, the engineered binding agent binds to the N-terminally functionalized target peptide with a thermodynamic dissociation constant (Kd) of 200 nM or less. In some preferred embodiments, the engineered binding agent binds to the N-terminally functionalized target peptide with a thermodynamic dissociation constant (Kd) of 100 nM or less.

In preferred embodiments, the signal (e.g., length and/or sequence of RNA transcript transcribed from the recording tag using a specific promoter) can be generated only when the RNAP portion A and the RNAP portion B are in sufficient proximity; this solves the problem of unspecific attachment of the binding agent to the solid support that would result in background noise. Having the disclosed split RNAP components, output signal is not significantly generated unless the cognate binding agent recognizes the polypeptide and brings the first RNAP portion A and the RNAP portion Bs into sufficient proximity to generate a detectable output RNA transcript.

Provided herein are methods for analyzing a macromolecule, such as a polypeptide, comprising providing a polypeptide and an associated RNAP portion A attached to a solid support; contacting the polypeptide with a binding agent capable of binding to the polypeptide, wherein the binding agent is associated with an RNAP portion B, whereby binding between the polypeptide and the binding agent brings the RNAP portion A and the RNAP portion B into sufficient proximity to interact with each other and generate a functional RNAP enzyme; and detecting a generated signal (RNA transcript). In some embodiments, the contacting of the polypeptide with a binding agent capable of binding to the polypeptide and detecting the signal is repeated sequentially one or more times. In some embodiments, a plurality of binding agents is allowed to contact a plurality of polypeptides for analysis. The plurality of binding agents may include a mixture of binding agents, at least some of which are associated with an RNAP portion B. In preferred embodiments, the methods described herein are performed on polypeptide(s) immobilized on a surface, e.g., any suitable material, including porous and non-porous materials, a planar surface, etc.

In preferred embodiments, the provided methods allow control over the generation of the detectable signal (RNA transcripts). For example, a detectable signal is not generated until a particular component is added to the sample being analysed. In some embodiments, this particular component may be one required for functional RNAP enzyme to interact with a specific promoter and produce RNA transcript (such as a buffer component, an ATP molecule, or a nucleotide). In some embodiments, the methods described herein can be applied for identifying one or more binding events between a plurality of binding agents and a plurality of polypeptides immobilized on a solid support. Identifying one or more binding events by the methods described herein provides a higher signal-to-noise ratio than that generated by other methods known in the art, since utilizing the described two-component detection agents (i.e. bimolecular or split RNAP components) offers reduced non-specific background noise, since binding agents unspecifically bound to the solid support are unable to generate a detectable signal.

In some embodiments, the disclosed methods outline how to couple the binding of a target (e.g., an epitope or an N-terminal amino acid (NTAA) residue of a polypeptide (P1)) by binding agents to promoter-specific RNA transcription, followed by direct RNA sequencing or reverse transcription to achieve sequenceable cDNA for next-generation sequencing.

In some embodiments, the solid support used for immobilization of a polypeptide in the claimed methods does not comprise polypeptide(s). In some embodiments, solid support used for immobilization of a polypeptide in the claimed methods does not comprise polynucleotide(s).

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.”

The term “antibody” herein is used in the broadest sense and includes polyclonal and monoclonal antibodies, including intact antibodies and functional (antigen-binding) antibody fragments, including fragment antigen binding (Fab) fragments, F(ab)2 fragments, Fab′ fragments, FAT fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins. Unless otherwise stated, the term “antibody” should be understood to encompass functional antibody fragments thereof.

As used herein, the term “sample” refers to anything which may contain an analyte for which an analyte assay is desired. As used herein, a “sample” can be a solution, a suspension, liquid, powder, a paste, aqueous, non-aqueous or any combination thereof. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, the term “macromolecule” encompasses large molecules composed of smaller subunits. Examples of macromolecules include, but are not limited to peptides, polypeptides, proteins, nucleic acids, carbohydrates, lipids, macrocycles, or a combination or complex thereof. A macromolecule also includes a chimeric macromolecule composed of a combination of two or more types of macromolecules, covalently linked together (e.g., a peptide linked to a nucleic acid). A macromolecule may also include a “macromolecule assembly”, which is composed of non-covalent complexes of two or more macromolecules.

As used herein, the term “macromolecule” encompasses large molecules composed of smaller subunits. Examples of macromolecules include, but are not limited to peptides, polypeptides, proteins, nucleic acids, carbohydrates, lipids, macrocycles. A macromolecule also includes a chimeric macromolecule composed of a combination of two or more types of macromolecules, covalently linked together (e.g., a peptide linked to a nucleic acid). A macromolecule may also include a “macromolecule assembly”, which is composed of non-covalent complexes of two or more macromolecules. A macromolecule assembly may be composed of the same type of macromolecule (e.g., protein-protein) or of two more different types of macromolecules (e.g., protein-DNA).

Herein, the term “polypeptide” is used interchangeably with the term “peptide”, and encompasses peptides and proteins, referring to a molecule comprising a chain of three or more amino acids joined by peptide bonds. In some embodiments, a polypeptide comprises 4 to 50 amino acids. In some embodiments, a peptide does not comprise a secondary, tertiary, or higher structure. In some embodiments, the polypeptide is a protein. In some embodiments, a protein comprises more than 50 amino acids. In some embodiments, in addition to a primary structure, a protein comprises a secondary, tertiary, or higher structure. The amino acids of the polypeptides are most typically L-amino acids, but may also be D-amino acids, modified amino acids, amino acid analogs, amino acid mimetics, or any combination thereof. Polypeptides may be naturally occurring, synthetically produced, or recombinantly expressed. Polypeptides may also comprise additional groups modifying the amino acid chain, for example, functional groups added via post-translational modification. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a labeling component.

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, (3-amino acids, Homo-amino acids, Proline and Pyruvic acid derivatives, 3-substituted alanine derivatives, glycine derivatives, ring-substituted phenylalanine and tyrosine derivatives, linear core amino acids, N-methyl amino acids.

As used herein, the term “binding agent” refers to a nucleic acid molecule, a polypeptide, a carbohydrate, or a small molecule that binds to, associates, unites with, recognizes, or combines with a binding target, e.g., a macromolecule analyte or a component or feature of a macromolecule analyte. In some embodiments, a binding agent comprises a polypeptide. In some embodiments, a binding agent comprises an aptamer. In some embodiments, a binding agent does not comprise a polynucleotide. In some embodiments, a binding agent forms a covalent association with the macromolecule analyte or component or feature of a macromolecule analyte. In other embodiments, a binding agent form a non-covalent association with the macromolecule analyte or component or feature of a macromolecule analyte. A binding agent may also be a chimeric binding agent, composed of two or more types of molecules, such as a nucleic acid molecule-peptide chimeric binding agent or a carbohydrate-peptide chimeric binding agent. A binding agent may be a naturally occurring, synthetically produced, or recombinantly expressed molecule. A binding agent may bind to a linear molecule or a molecule having a three-dimensional structure (also referred to as conformation). For example, an antibody binding agent may bind to linear peptide, polypeptide, or protein, or bind to a conformational peptide. A binding agent may bind to an N-terminal peptide, a C-terminal peptide, or an intervening peptide of a peptide, polypeptide, or protein molecule. A binding agent may bind to an N-terminal amino acid, C-terminal amino acid, or an intervening amino acid of a peptide molecule. A binding agent may preferably bind to a chemically functionalized or modified amino acid (e.g., an amino acid that has been functionalized or modified by a functionalizing reagent) over a non-modified amino acid. For example, a binding agent may preferably bind to an amino acid that has been functionalized or modified over an amino acid that is unmodified. A binding agent may bind to a post-translational modification of a peptide molecule. A binding agent may exhibit selective binding to a component or feature of a polypeptide or modified polypeptide (e.g., a binding agent may selectively bind to one of the 20 possible natural amino acid residues and bind with very low affinity or not at all to the other 19 natural amino acid residues). A binding agent may exhibit less selective binding, where the binding agent is capable of binding or configured to bind to a plurality of components or features of a polypeptide (e.g., a binding agent may bind with similar affinity to two or more different amino acid residues).

The terms “specific binding” generally refers to an engineered binding agent that binds to a particular functionalized amino acid residue more readily than it would bind to a random functionalized amino acid residue (e.g., there is a detectable relative increase in the binding of the binding agent to a specific or group of functionalized amino acid residues). The term “specificity” is used herein to qualify the relative affinity by which an engineered binding agent binds to a cognate functionalized amino acid residue. Specific binding typically means that an engineered binding agent binds to a cognate functionalized amino acid residue at least twice more likely that to a random, non-cognate functionalized amino acid residue (a 2:1 ratio of specific to non-specific binding). Non-specific binding or unspecific binding refers to background binding, and is the amount of signal that is produced in a binding assay between an engineered binding agent and a non-cognate amino acid residue or residues immobilized on a solid support. In some embodiments, specific binding refers to binding between an engineered binding agent and a cognate functionalized amino acid residue with a thermodynamic dissociation constant (Kd) of 500 nM or less.

As used herein, the term “detectable label” refers to a substance which can indicate the presence of another substance when associated with it. The detectable label can be a substance that is linked to or incorporated into the substance to be detected. In some embodiments, a detectable label is suitable for allowing for detection and also quantification, for example, a detectable label that emits a detectable and measurable signal. Detectable labels include any labels that can be utilized and are compatible with the provided macromolecule analysis assay format and include, but are not limited to, a bioluminescent label, a RNA transcript, a biotin/avidin label, a chemiluminescent label, a chromophore, a coenzyme, a dye, an electro-active group, an electrochemiluminescent label, an enzymatic label (e.g., alkaline phosphatase, luciferase or horseradish peroxidase), a fluorescent label, a latex particle, a magnetic particle, a metal, a metal chelate, a phosphorescent dye, a protein label, a radioactive element or moiety, and a stable radical.

As used herein, the term “linker” refers to one or more of a nucleotide, a nucleotide analog, an amino acid, a peptide, a polypeptide, a polymer, or a non-nucleotide chemical moiety that is used to join two molecules. A linker may be used to associate an RNAP portion A with a polypeptide, a binding agent with an RNAP portion B, a polypeptide with a solid support, a detection agent with a solid support, etc. A linker may be used to join a DNA tag (e.g., a recording tag) with a polypeptide or a DNA tag with a solid support. In certain embodiments, a linker joins two molecules via enzymatic reaction or chemistry reaction (e.g., click chemistry).

The term “ligand” as used herein refers to any molecule or moiety connected to the compounds described herein. “Ligand” may refer to one or more ligands attached to a compound. In some embodiments, the ligand is a pendant group or binding site (e.g., the site to which the binding agent binds).

The terminal amino acid at one end of a peptide or polypeptide chain that has a free amino group is referred to herein as the “N-terminal amino acid” (NTAA or P1). The terminal amino acid at the other end of the chain that has a free carboxyl group is referred to herein as the “C-terminal amino acid” (CTAA). The amino acids making up a peptide may be numbered in order, with the peptide being “n” amino acids in length. As used herein, NTAA is considered the n^{th} amino acid (also referred to herein as the “n NTAA”). Using this nomenclature, the next amino acid is the n−1 amino acid, then the n−2 amino acid, and so on down the length of the peptide from the N-terminal end to C-terminal end. In certain embodiments, an NTAA, CTAA, or both may be modified or functionalized with a moiety or a chemical moiety.

As used herein, the term “barcode” refers to a molecule providing a unique identifier tag or origin information for a polypeptide, a binding agent, a sample polypeptides, a set of samples, polypeptides within a compartment (e.g., droplet, bead, or separated location), polypeptides within a set of compartments, a fraction of polypeptides, a set of polypeptide fractions, a spatial region or set of spatial regions, a library of polypeptides, or a library of binding agents. As an example, polypeptide (or peptide) analyte-specific barcode identifies and/or distinguishes a specific peptide analyte from other polypeptide (or peptide) analytes. A “nucleic acid barcode” refers to a nucleic acid molecule of about 3 to about 30 bases (e.g., 3, 4, 5, 6, 7, 8, 9, 10, 11, 12, 13, 14, 15, 16, 17, 18, 19, 20, 21, 22, 23, 24, 25, 26, 27, 28, 29 or 30 bases). A “peptide barcode” or “amino acid barcode” refers to a sequence of amino acids that can have a length of at least, for example, 2, 3, 4, 5, 6, 7, 8, 9, 10, 11, 12, 13, 14, 15, 16, 17, 18, 19, 20, 21, 22, 23, 24, 25, 30, 40, 50, 75, or 100 amino acids. A specific peptide barcode can be distinguished from other peptide barcodes by having a different length, sequence, or other physical property (for example, hydrophobicity). A barcode can be an artificial sequence or a naturally occurring sequence. In certain embodiments, each barcode within a population of barcodes is different. In other embodiments, a portion of barcodes in a population of barcodes is different, e.g., at least about 10%, 50%, 70%, 75%, 80%, 85%, 90%, 95%, 97%, or 99% of the barcodes in a population of barcodes is different. A population of barcodes may be randomly generated or non-randomly generated. In certain embodiments, a population of barcodes are error-correcting or error-tolerant barcodes. Barcodes can be used to computationally deconvolute the multiplexed sequencing data and identify sequence reads derived from an individual polypeptide, sample, library, etc.

As used herein, the term “primer extension”, also referred to as “polymerase extension”, refers to a reaction catalyzed by a nucleic acid polymerase (e.g., DNA polymerase) whereby a nucleic acid molecule (e.g., oligonucleotide primer, spacer sequence) that anneals to a complementary strand is extended by the polymerase, using the complementary strand as template.

As used herein, the term “unique molecular identifier” or “UMI” refers to a nucleic acid molecule of about 3 to about 40 bases in length providing a unique identifier tag for each macromolecule, polypeptide or binding agent to which the UMI is linked. A polypeptide UMI can be used to accurately count originating polypeptide molecules by collapsing NGS reads to unique UMIs. A binding agent UMI can be used to identify each individual molecular binding agent that binds to a particular polypeptide. For example, a UMI can be used to identify the number of individual binding events for a binding agent specific for a single amino acid that occurs for a particular peptide molecule. It is understood that when UMI and barcode are both referenced in the context of a binding agent or polypeptide, that the barcode refers to identifying information other that the UMI for the individual binding agent or polypeptide (e.g., sample barcode, compartment barcode, binding cycle barcode).

As used herein, the terms “solid support”, “solid surface”, and “support”, which are used interchangeably, refer to any solid material, including porous and non-porous materials, to which a macromolecule can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, a flow cell, a biochip including signal transducing electronics, a channel, a microtiter well, an ELISA plate, a polymer matrix, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), dextran, or any combination thereof. For example, when solid surface is a bead, the bead can include, but is not limited to, a ceramic bead, a polystyrene bead, a polymer bead, a polyacrylate bead, an agarose bead, a cellulose bead, a dextran bead, an acrylamide bead, a porous bead, or any combinations thereof. A bead may be spherical or an irregularly shaped. A bead or support may be porous. A bead's size may range from nanometers, e.g., 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 5, 10, or 20 μm in diameter. In certain embodiments, “a bead” solid support may refer to an individual bead or a plurality of beads. In some embodiments, the solid surface is a nanoparticle. In certain embodiments, the nanoparticles range in size from about 1 nm to about 500 nm in diameter. In some embodiments, the nanoparticles are less than about 200 nm in diameter.

As used herein, the term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), γPNAs, morpholino polynucleotides, locked nucleic acids (LNAs). In some embodiments, the nucleic acid molecule or oligonucleotide is a modified oligonucleotide. In some embodiments, the nucleic acid molecule or oligonucleotide is a DNA with pseudo-complementary bases, a DNA with protected bases, an RNA molecule, a BNA molecule, an XNA molecule, a LNA molecule, a PNA molecule, a γPNA molecule, or a morpholino DNA, or a combination thereof. In some embodiments, the nucleic acid molecule or oligonucleotide is backbone modified, sugar modified, or nucleobase modified.

As used herein, “nucleic acid sequencing” means the determination of the order of nucleotides in a nucleic acid molecule or a sample of nucleic acid molecules. Similarly, “polypeptide sequencing” means the determination of the identity and order of at least a portion of amino acids in the polypeptide molecule or in a sample of polypeptide molecules.

As used herein, “next generation sequencing” refers to high-throughput sequencing methods that allow the sequencing of millions to billions of molecules in parallel. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high-throughput nucleic acid sequencing technology include platforms provided by Illumina, BGI, Qiagen, Thermo-Fisher, and Roche.

As used herein, “analyzing” the macromolecule means to identify, detect, quantify, characterize, distinguish, or a combination thereof, all or a portion of the components of the macromolecule. For example, analyzing a peptide includes determining all or a portion of the amino acid sequence (contiguous or non-continuous) of the peptide. Analyzing a polypeptide also includes partial identification of a component of the polypeptide. For example, partial identification of amino acids in the polypeptide protein sequence can identify an amino acid in the protein as belonging to a subset of possible amino acids. Analysis typically begins with analysis of the n NTAA, and then proceeds to the next amino acid of the peptide (i.e., n−1, n−2, n−3, and so forth). This is accomplished by elimination of the n NTAA, thereby converting the n−1 amino acid of the peptide to an N-terminal amino acid (referred to herein as the “n−1 NTAA”). Analyzing the peptide may also include determining the presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

The term “unmodified” (also “wild-type” or “native”) as used herein is used in connection with biological materials such as nucleic acid molecules and proteins, refers to those which are found in nature and not modified by human intervention.

As used herein, a polynucleotide or polypeptide variant, mutant, homologue, or modified version include polynucleotides or polypeptides that share nucleic acid or amino acid sequence identity with a reference polynucleotide or polypeptide. For example, variant or modified polypeptide generally exhibits about 25%, 30%, 40%, 50%, 60%, 70%, 80%, 85%, 86%, 87%, 88%, 89%, 90%, 91%, 92%, 93%, 94%, 95%, 96%, 97%, 98%, 99% or more sequence identity to a corresponding wild-type or unmodified polypeptide. The term “modified” or “engineered” (or “variant” or mutant”) as used in reference to polynucleotides and polypeptides implies that such molecules are created by human intervention and/or they are non-naturally occurring. A variant, mutant or modified polypeptide is not limited to any variant, mutant or modified polypeptide made or generated by a particular method of making and includes, for example, a variant, mutant or modified polypeptide made or generated by genetic selection, protein engineering, directed evolution, de novo recombinant DNA techniques, or combinations thereof. A mutant, variant or modified polypeptide is altered in primary amino acid sequence by substitution, addition, or deletion of amino acid residues. In some embodiments, variants of a polypeptide (e.g., an RNAP portion B) displaying only non-substantial or negligible differences in structure can be generated by making conservative amino acid substitutions in the modified polypeptide. By doing this, modified polypeptide variants that comprise a sequence having at least 80% (at least 85%, 90%, 91%, 92%, 93%, 94%, 95%, 96%, 97%, 98%, or 99%) sequence identity with the corresponding unmodified polypeptide sequence can be generated, retaining at least one functional activity of the polypeptide. Examples of conservative amino acid changes are known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; leucine to valine or isoleucine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Examples of non-conservative amino acid changes that are likely to cause major changes in protein structure or function are those, for example, that cause substitution of a hydrophilic residue to a hydrophobic residue. Methods of making targeted amino acid substitutions, deletions, truncations, and insertions are generally known in the art. For example, amino acid sequence variants can be prepared by mutations in the DNA. Methods for polynucleotide alterations are well known in the art, for example, Kunkel et al. (1987) Methods in Enzymol. 154:367-382; U.S. Pat. No. 4,873,192 and the references cited therein.

The term “sequence identity” is a measure of identity between polypeptides at the amino acid level, and a measure of identity between nucleic acids at nucleotide level. The polypeptide sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. “Sequence identity” means the percentage of identical subunits at corresponding positions in two sequences when the two sequences are aligned to maximize subunit matching, i.e., taking into account gaps and insertions. For example, the BLAST algorithm (NCBI) calculates percent sequence identity and performs a statistical analysis of the similarity and identity between the two sequences. The software for performing BLAST analysis is publicly available through the National Center for Biotechnology Information (NCBI) website.

The terms “corresponding to position(s)” or “position(s) . . . with reference to position(s)” of or within a polypeptide or a polynucleotide, such as recitation that nucleotides or amino acid positions “correspond to” nucleotides or amino acid positions of a disclosed sequence, such sequence set forth in the Sequence Listing, refers to nucleotides or amino acid positions identified in the polynucleotide or in the polypeptide upon alignment with the disclosed sequence using a standard alignment algorithm, such as the BLAST algorithm (NCBI). One skilled in the art can identify any given amino acid residue in a given polypeptide at a position corresponding to a particular position of a reference sequence, such as set forth in the Sequence Listing, by performing alignment of the polypeptide sequence with the reference sequence (for example, by using BLASTP publicly available through the NCBI website), matching the corresponding position of the reference sequence with the position in polypeptide sequence and thus identifying the amino acid residue within the polypeptide.

The term “joining” or “attaching” one substance to another substance means connecting or linking these substances together utilizing one or more covalent bond(s) and/or non-covalent interactions. Some examples of non-covalent interactions include hydrogen bonding, hydrophobic binding, and Van der Waals forces. Joining can be direct or indirect, such as via a linker or via another moiety. In preferred embodiments, joining two or more substances together would not impair structure or functional activities of the joined substances. The term “associated with” (e.g., one substance is associated with to another substance) means bringing two substances together, so they can coordinately participate in the methods described herein. In preferred embodiments, association of two substances preserves their structures and functional activities. Association can be direct or indirect. When one substance is directly associated with another substance, it is equivalent to one substance being joined or attached to another substance. Indirect association means that two substances are brought together by means other than direct joining or attachment. For example, in some embodiments, the macromolecule may be associated with the RNAP portion A via a solid support and one or more linkers (both the macromolecule and the RNAP portion A are independently attached to the solid support). In some embodiments, indirect association implies that two substances are co-localized with each other, or located in a close proximity with each other.

As used herein, the term “macromolecule comprises a component” refers to a situation where the component is either a part of the macromolecule, or directly attached to the macromolecule by means of one or more covalent bond(s), which unite them into a single molecule. Instead, the term “macromolecule associated with a component” indicates that the component may or may not be directly attached to the macromolecule by means of one or more covalent bond(s), but instead can be associated, or co-localized, with the macromolecule by means of non-covalent interactions, or, alternatively, be associated indirectly through a solid support (for example, when the macromolecule is attached to the solid support, and the component is independently attached to the solid support in a proximity to the macromolecule. For example, “macromolecule is associated with a recording tag” encompasses various possible ways for association between the macromolecule and the recording tag (either direct, covalent or non-covalent association, or indirect association, such as association via a linker or via another object, such as via solid support). The terms “attaching” and “joining” are used interchangeably and refer to either covalent or non-covalent attachment.

As used herein, the term “releasably attached” refers to covalent or non-covalent attachment of a component (such as the RNAP portion A) to a solid support, wherein such attachment can be reversed under mild conditions, such as conditions that do not impair or compromise structure or functional activity of this component and other components of the system or complex.

As used herein, “RNA polymerase (RNAP) portion A” and “RNA polymerase (RNAP) portion B” are two components of a functional RNA polymerase (RNAP) enzyme, and refer to an RNAP enzyme that is assembled from at least two fragments, named an RNAP portion A and an RNAP portion B, to form a functional RNAP enzyme capable of binding to a DNA promoter and initiating transcription of a single RNA transcript from the DNA downstream of the promoter. In preferred embodiments, a RNAP portion A and a RNAP portion B being in proximity (i.e., they are sufficiently close to each other) spontaneously assemble to form a functional RNAP enzyme. In preferred embodiments, the functional RNAP enzyme is a single-subunit DNA-dependent RNA polymerase. In preferred embodiments, an individual RNAP portion A and an individual RNAP portion B are unfunctional and/or are enzymatically inactive, such as when an RNAP portion A and an RNAP portion B are not in sufficient proximity to interact with each other, these fragments are not capable of binding to a promoter and/or initiating transcription of a RNA transcript from the DNA downstream of the promoter. In preferred embodiments, formation of a functional RNAP enzyme from an RNAP portion A and an RNAP portion B is proximity-dependent.

Exemplary RNAP portion As, RNAP portion Bs, and functional RNAP enzymes are disclosed in the following patent publications: US2020/0332371, US2015/0368625 and US2020/0199599, incorporated herein by reference, as well as in the following publications: Pu J, et al., 2017; Pu J, et al., J Am Chem Soc. 2017; Temme K, et al., 2012; Meyer A J, et al., 2015.

As used herein, the term “transcription” refers to the process by which a functional RNAP enzyme produces an RNA copy (“RNA transcript”) of a piece of DNA molecule (copying a segment of a DNA molecule into a RNA molecule).

As used herein, the term “T7-related RNA polymerase” refers to a functional RNA polymerase enzyme capable of binding a DNA promoter and initiating synthesis of an RNA molecule (called “RNA transcript”) from the DNA promoter, wherein the RNA polymerase comprises an amino acid sequence having at least 30% sequence identity to any one of the amino acid sequences set forth in SEQ ID NO: 1-SEQ ID NO: 4.

As used herein, the term “orthogonal” refers to two or more RNAP portion Bs or to two or more functional RNAPs, wherein each of the RNAP portion Bs or functional RNAPs displays a robust activity on its target promoter, but at the same time displays a significantly reduced activity (e.g., less than 20% activity, less than 10% activity, less than 5% activity, or less than 1% activity) on the other (non-cognate) promoters that are cognate to one or more of the other RNAP portion Bs or the other functional RNAPs. For example, RNAPs having amino acid sequences set forth in SEQ ID NO: 1-SEQ ID NO: 4 are orthogonal, because each of the RNAPs initiates transcription from the cognate promoter (such as T7 promoter, T3 promoter, K11 promoter and SP6 promoter, correspondingly), but at the same time is unable to initiate (or initiates with much less efficiency) transcription from the non-cognate promoters (e.g., RNAPs having amino acid sequence set forth in SEQ ID NO: 1 is unable to initiate transcription from T3 promoter, K11 promoter, or SP6 promoter). Orthogonality can be achieved by engineering RNAP portion Bs of functional RNAPs to bind to different promoters and not cross-react. Exemplary sets of orthogonal RNAP portion Bs are recited in Examples 2-4. Similarly, “orthogonal pairs” refers to two or more pairs, wherein each pair consists of a functional RNAP and a cognate promoter, and functional RNAPs from different pairs are orthogonal.

As used herein, the term “recording tag” refers to a one or more nucleic acid molecules, such as one or more single stranded, double stranded (or combination of single stranded and double stranded) DNA molecules, associated with a macromolecule analyte and comprising a plurality of promoters. As used herein, “promoter” is a short region of a nucleic acid (e.g., 20-200 bp) to which functional RNA polymerase (RNAP) enzyme can bind to initiate transcription of a single RNA transcript from the nucleic acid (e.g., DNA, or modified DNA) downstream of the promoter. In some embodiments, recording tag also comprises an analyte-specific barcode or UMI, such as macromolecule analyte-specific barcode or UMI. In some embodiments, when at least 100, at least 500, at least 1000 or more different macromolecule analytes are analyzed simultaneously, each recording tag associated with one of the at least 100, at least 500, at least 1000 or more different macromolecule analytes comprises a different (unique) analyte-specific barcode or UMI. In some embodiments, recording tag associated with a macromolecule analyte consists of a single DNA molecule, which comprises the plurality of promoters. In other embodiments, recording tag associated with a macromolecule analyte comprise a plurality of DNA molecules attached to the solid support, wherein each DNA molecule of the plurality of DNA molecules comprises a different promoter from a plurality of promoters. A recording tag may further comprise other functional components, e.g., a universal priming site, an UMI, a barcode (e.g., a sample barcode, a fraction barcode, spatial barcode, a compartment tag, etc.), a spacer sequence or any combination thereof.

It is understood that aspects and embodiments of the invention described herein include “consisting of” and/or “consisting essentially of” aspects and embodiments.

Throughout this disclosure, various embodiments of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

Provided herein are methods for analyzing a macromolecule analyte, comprising the steps of:
(a) providing the macromolecule analyte attached to a solid support, wherein the macromolecule analyte is associated with (i) an RNAP portion A, and (ii) a recording tag comprising a plurality of promoters, and optionally comprising an analyte-specific barcode;
(b) contacting the macromolecule analyte with a plurality of binding agents capable of binding to the macromolecule analyte, wherein each binding agent of the plurality of binding agents is joined to an RNAP portion B, whereby binding between the macromolecule analyte and a first binding agent of the plurality of binding agents brings the RNAP portion A and an RNAP portion B joined to the first binding agent into sufficient proximity to interact with each other and form a first functional RNAP enzyme configured to initiate transcription from a first promoter of the plurality of promoters;
(c) following binding of the first binding agent to the macromolecule analyte, generating a first RNA transcript by the first functional RNAP enzyme from the recording tag associated with the macromolecule analyte;
(d) collecting the first RNA transcript, and disrupting the first functional RNAP enzyme or washing the first functional RNAP enzyme away from the recording tag associated with the macromolecule analyte;
(e) optionally, removing a portion of the macromolecule analyte;
(f) repeating steps (b), (c), (d) and, optionally, (e) sequentially one or more times by replacing the first binding agent with a second or higher order binding agent capable of binding to the macromolecule analyte, wherein binding between the macromolecule analyte and a second or higher order binding agent of the plurality of binding agents brings the RNAP portion A and an RNAP portion B joined to the second or higher order binding agent into sufficient proximity to interact with each other and form a second or higher order functional RNAP enzyme configured to initiate transcription from a second or higher order promoter of the plurality of promoters, thereby generating and collecting a second and, optionally, higher order RNA transcript; and
(g) analyzing the first, the second and, optionally, the higher order RNA transcripts, which comprises identifying RNA transcript lengths and/or at least partial sequences of the first, the second and, optionally, the higher order RNA transcripts, thereby analyzing the macromolecule analyte.

Also disclosed herein is a system for analyzing a plurality of macromolecule analytes, comprising:
a) the plurality of macromolecule analytes attached to a solid support, wherein each macromolecule analyte from the plurality of macromolecule analytes is associated with (i) an RNAP portion A, and (ii) a recording tag comprising a plurality of promoters, and optionally comprising an analyte-specific barcode; and
b) a plurality of binding agents capable of binding to the macromolecule analyte, wherein each binding agent of the plurality of binding agents is joined to an RNAP portion B, whereby binding between the macromolecule analyte and a first binding agent of the plurality of binding agents brings the RNAP portion A and an RNAP portion B joined to the first binding agent into sufficient proximity to interact with each other and form a first functional RNAP enzyme configured to initiate transcription from a first promoter of the plurality of promoters.

In preferred embodiments of the disclosed methods, the following steps are repeated sequentially one or more times: (i) contacting the macromolecule analyte with the plurality of binding agents and generating a functional RNAP enzyme, (ii) generating a RNA transcript by the functional RNAP enzyme from the recording tag associated with the macromolecule analyte, (iii) collecting the RNA transcript, and disrupting the functional RNAP enzyme or washing the functional RNAP enzyme away from the recording tag, and, optionally, (iv) removing a portion (such as a functionalized NTAA) of the macromolecule analyte. In some embodiments of the disclosed methods, these steps are repeated 2, 3, 4, 5, 6, 7, 8, 9, 10, 15, 20, 30 or more times. The steps (i)-(iii) (optionally, (i)-(iv)) form a binding cycle, where a macromolecule analyte is contacted with a single binding agent, which leads to formation of a functional RNAP enzyme, followed by generation of a specific RNA transcript, which is collected and may be analyzed or stored for the later analysis. After collecting the RNA transcripts, removing the RNAP enzyme away from the system, and, optionally, removing a portion of the macromolecule analyte, the next binding cycle starts by contacting the same macromolecule analyte (or the same macromolecule analyte without the removed portion) with a plurality of binding agents (step (i)) followed by the corresponding steps (ii), (iii), and optionally, (iv). At each binding cycle, a productive interaction between the macromolecule analyte and the binding agent results in formation of a functional RNAP enzyme, which generates a unique RNA transcript.

In preferred embodiments of the disclosed methods, at step (c) at least several copies of the first RNA transcript are generated. Similarly, in some embodiments, at least several copies of the second or higher order RNA transcript are generated. In some embodiments, at least 2, at least 5, at least 10, at least 15, or at least 20 copies of the first, the second or higher order RNA transcript are generated. Generating more copies of the first, second or higher order RNA transcripts provides a higher signal-to-noise ratio for detecting corresponding interactions between the macromolecule analyte and the binding agents. RNA transcripts can be further amplified during RT-PCR (e.g., when a cDNA Sequencing Kit is used to identify sequences of RNA transcripts), which further amplifies the detectable signal for the disclosed methods.

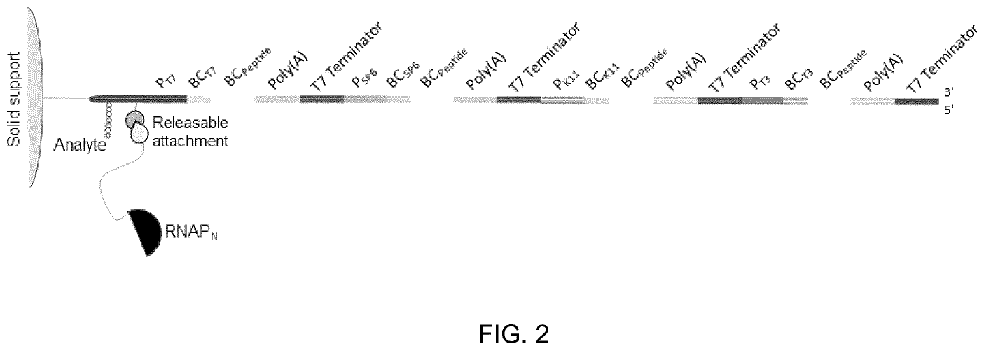

In some embodiments of the disclosed methods, during the analyzing step (g), RNA transcript lengths are evaluated, rather than sequences of generated RNA transcripts. For example, when the recording tag associated with the macromolecule analyte consists of a single DNA sequence, which comprises the plurality of promoters, RNA transcripts generated by a functional RNAP enzyme from the recording tag have different lengths depending on which promoter from the plurality of promoters is a binding substrate to the functional RNAP enzyme (see, e.g., FIG. 1D). Information regarding lengths of the RNA transcripts generated at each binding cycle can be used to provide the identity of the binding agents that were bound to the macromolecule analyte at each binding cycle. In other embodiments of the disclosed methods, information regarding nucleotide sequences of the RNA transcripts generated at each binding cycle can be used to provide identity of the binding agents that were bound to the macromolecule analyte at each binding cycle (see, e.g., FIGS. 2-4), in which a promoter-specific barcode is transcribed onto the synthesized RNA transcript

In some embodiments of the disclosed methods, binding agent-RNAPC fusion concentrations are fixed at their on-target P1 Kd (dissociation constant for the P1 residue of peptide analytes) enabling uniform encoding rates per reaction site.

In other embodiments of the disclosed methods, information regarding nucleotide sequences of the RNA transcripts generated at each binding cycle can be used to provide identity of the binding agents that were bound to the macromolecule analyte at each binding cycle. In some embodiments, in the recording tag, any one or more of the different promoters can be used to generate a transcript comprising a promoter-specific barcode, for instance, promoter-specific barcode BCT3 for promoter PT3, etc., as shown in FIGS. 2-4. The recording tag shown in FIGS. 1A-C may also comprise one or more promoter-specific barcodes, such that RNA transcript lengths and/or sequence can be evaluated to reveal which promoter in the plurality of promoters is used for transcription.

In some embodiments of the disclosed methods and systems, 100, 500, 1000, 10000, 100000 or more different macromolecule analytes are analyzed simultaneously, and each recording tag associated with one of the 100, 500, 1000, 10000, 100000 or more different macromolecule analytes comprises a different analyte-specific barcode.

In some embodiments, the plurality of promoters comprises at least 3, 4, 5, 6, 7, 8, 9, 10, or more different promoters. In some preferred embodiments, the plurality of promoters comprises at least four different promoters.

In some embodiments, at least four binding agents from the plurality of binding agents have different RNAP portion Bs, and at least four different functional RNAP enzymes are formed via interactions between the RNAP portion A and the four different RNAP portion Bs joined to the at least four binding agents, wherein each of the at least four different functional RNAP enzymes have different binding specificities to promoters from the plurality of promoters.

In some embodiments, the RNAP portion A is not covalently attached to the macromolecule analyte. In such a case, RNAP portion A may be removed in step (d) or (e), and added back before step (f), which may prevent the RNAP portion A from being chemically modified or enzymatically modified during optional NTAA functionalization or optional NTAA cleavage.

In some embodiments, the disclosed methods further comprise step (e): removing a portion of the macromolecule analyte, which is performed before repeating steps (b), (c), and (d). This allows for sequential sequence and/or structural analysis of the macromolecule analyte via two or more cycles. In some embodiments of the disclosed methods and systems, at least the first, the second and/or the higher order binding agents are capable of binding to an N-terminal amino acid (NTAA) of the polypeptide analyte, or is capable of binding to an N-terminal amino acid (NTAA) of the polypeptide analyte functionalized with a chemical moiety. In some of these embodiments, the NTAA identity is encoded in a sequence of RNA transcripts that are generated after binding of the first, the second and/or the higher order binding agents to the NTAA or functionalized NTAA. In some of these embodiments, the portion of the polypeptide analyte removed at step (e) comprises the NTAA, or the functionalized NTAA. After cleavage, a newly exposed NTAA of the polypeptide analyte is generated, and the steps of contacting, generating one or more RNA transcripts collecting the RNA transcripts, and optionally, removing the NTAA or functionalized NTAA are repeated one or more times. Where binding agents capable of binding to functionalized NTAAs are used, a step of contacting the immobilized polypeptide analyte with a functionalizing reagent capable of modifying an N-terminal amino acid (NTAA) residue of the polypeptide analyte to generate a functionalized NTAA residue is performed before the contacting step (b), and is repeated during step (f).

In some embodiments, the binding between the macromolecule, such as polypeptide, and the binding agent is reversible. For example, the binding agent may be released or removed from the macromolecule. In some embodiments, the NTAA is removed from the polypeptide after the signal (RNA transcript) is generated, thereby yielding a newly exposed NTAA, and the above steps are repeated on the newly exposed NTAA.

In preferred embodiments of the disclosed methods, upon binding between a first motif of the macromolecule analyte and the binding moiety of a first binding agent of the plurality of binding agents, the RNAP portion A associated with the macromolecule analyte and the RNAP portion B of the first binding agent are brought into proximity to associate and form a first functionally reconstituted RNAP enzyme, which initiates transcription specifically from a corresponding first promoter of the plurality of promoters to synthesize a first ribonucleic acid (RNA) transcript released from the support. Similarly, upon binding between a second motif of the macromolecule analyte and the binding moiety of a second binding agent of the plurality of binding agents, the RNAP portion A associated with the macromolecule analyte and the RNAP portion B of the second binding agent are brought into proximity to associate and form a second functionally reconstituted RNAP enzyme, which initiates transcription specifically from a corresponding second promoter of the plurality of promoters to generate a second RNA transcript released from the support.

In some embodiments, the methods further comprise a step of contacting the immobilized polypeptide analyte with a functionalizing reagent capable of modifying an N-terminal amino acid (NTAA) residue of the polypeptide analyte to generate a functionalized NTAA residue. In some of these embodiments, the functionalized NTAA residues of immobilized polypeptide analytes are removed at the removing step (e). In some embodiments, the portion of the polypeptide analyte removed at step (e) comprises the NTAA modified with the chemical moiety, and the NTAA modified with the chemical moiety is removed from the polypeptide analyte by a modified cleavase enzyme.

In some embodiments, the modified cleavase enzyme: (i) is configured to cleave a peptide bond between a terminally labeled amino acid residue and a penultimate terminal amino acid residue of a polypeptide; (ii) is derived from a dipeptidyl aminopeptidase, which removes an unlabeled terminal dipeptide from a polypeptide; (iii) comprises two or more amino acid substitutions in the residues corresponding to positions N191, W/F192, R196, N306, and D650 of SEQ ID NO: 60, wherein the dipeptide aminopeptidase comprises an amino acid sequence having at least 20% sequence identity to the amino acid sequence of SEQ ID NO: 60 and also comprising an asparagine residue at a position corresponding to position 191 of SEQ ID NO: 60, a tryptophan residue or phenylalanine residue at a position corresponding to position 192 of SEQ ID NO: 60, an arginine residue at a position corresponding to position 196 of SEQ ID NO: 60, an asparagine residue at a position corresponding to position 306 of SEQ ID NO: 60, and an aspartate residue at a position corresponding to position 650 of SEQ ID NO: 60.

In preferred embodiments, modified cleavase enzymes are used at step (e) to remove the functionalized NTAA residues of immobilized polypeptide analytes, and the modified cleavase enzymes are disclosed in U.S. Pat. No. 11,427,814, incorporated herein by reference.

In some embodiments, the functionalized NTAA residues of immobilized polypeptide analytes are removed at step (e) by chemical methods disclosed in U.S patent publication No. 2022/0227889 A1, incorporated herein by reference.

In some embodiments, at step (g), the first, the second and, optionally, higher order RNA transcripts are analyzed by RNA or cDNA sequencing.

In some embodiments of the disclosed methods and systems, identities of the analyzed polypeptide analytes can be obtained. In some embodiments, at step (g), at least a portion of an amino acid sequence of the polypeptide analyte is identified. As used herein, “identifying” a polypeptide analyte comprises predicting identity of the polypeptide analyte with a certain probability. It can be done by identifying a component (e.g., one or more amino acid residues) of the polypeptide analyte. It can also be done by predicting several amino acid residues of the polypeptide analyte and their positions with certain probability, thus creating a polypeptide signature, and then matching bioinformatically the resulted peptide signature with corresponding signatures of polypeptides that may be present in the sample (e.g., by matching the polypeptide signature with polypeptide sequences from a proteomic or genomic database). For example, in some embodiments, existing selectivity of a binding agent is not enough to determine the NTAA residue to which the binding agent is bound with certainty. In these cases, identity of the NTAA residue can be determined with certain probability (such as being D, E or H and not A, G, I or L). Subsequent similar determination of adjacent amino acid residues creates an array of possible variants for the polypeptide analyte based on variants in the assayed amino acid residues, and by matching this array of variants with theoretical possibilities determined from a proteomic or genomic database, it can be narrowed down to a particular sequence, if enough amino acid residues were assayed with a certain probability.

In some embodiments, during the analyzing step, an artificial intelligence (AI) model is applied to calculate probabilities of occurrence of specific types of amino acid residues in corresponding places in amino acid sequence of the analyzed polypeptides based on collected nucleotide sequences of RNA transcripts.

In some embodiments, RNA transcripts collected during each binding cycle are analyzed by nanopore sequencing. For example, in some embodiments, RNA transcripts are analyzed by Direct RNA Sequencing Kit (SQK-Oxford Nanopore Technologies; RNA002). Alternatively, in some embodiments, RNA transcripts can be reverse transcribed (by, without limitation, RT-PCR) and then analyzed by Direct cDNA Sequencing Kit (Oxford Nanopore Technologies, SQK-DCS109) or PCR-cDNA Sequencing Kit (Oxford Nanopore Technologies, SQK-PCS109). In other embodiments, Illumina's RNA-SEQ (next-generation sequencing (NGS) methods for RNA sequencing) methods can be applied for sequencing of RNA transcripts collected during each binding cycle. In yet other embodiments, RNA transcripts are analyzed by any one of available RNA sequencing methods, such as by a method disclosed in Stark R, Grzelak M, Hadfield J. RNA sequencing: the teenage years. Nat Rev Genet. 2019 November; 20(11):631-656.

In some embodiments, the RNAP portion A is non-covalently attached to the solid support while associated with the macromolecule analyte. In some of these embodiments, the RNAP portion A associated with the macromolecule analyte is releasably or reversibly attached to the solid support. This design provides certain advantages for overall efficiency of the described methods, since it allows for replacing the RNAP portion A when necessary, such as before cleavage of NTAA residues of immobilized polypeptide analytes. In some embodiments, the RNAP portion A is releasably or reversibly attached to the solid support via a selectively cleavable linkage, such as linkage that can be disrupted or cleaved without disrupting or cleaving other linkages in components of the system (such as in conjugates, macromolecule analytes, solid support, etc.). In one embodiment, a dethiobiotin-modified RNAP portion A is attached to a streptavidin covalently coupled to the solid support. A biotin molecule can be used to displace the RNAP portion A, which can be re-attached in the next binding cycle. Other known covalent and non-covalent interaction partners can be used to releasably or reversibly attach the RNAP portion A to the solid support.

In some embodiments of the disclosed methods and systems, the first binding agent, the second binding agent and/or the higher order binding agent form(s) a non-covalent association with the macromolecule analyte. In other embodiments, the first binding agent, the second binding agent and/or the higher order binding agent form a covalent association with the macromolecule analyte. Covalent association provides opportunity for more efficient generation of the functional RNAP enzyme through interaction between the RNAP portion A and the RNAP portion B joined to the binding agent, since the RNAP fragments can stay in sufficient proximity with each other for a longer period of time. At the same time, covalent association provides opportunity for more stringent washes, which are preferable for removal of the functional RNAP enzyme and binding agents at each binding cycle.

In some embodiments, the first binding agent, the second binding agent and/or the higher order binding agent form a covalent reversible or selectively cleavable linkage with the macromolecule analyte, so that the binding between the macromolecule analyte and the binding agent can be disrupted after RNA transcripts are generated by the functional RNAP enzyme from the recording tag associated with the macromolecule analyte. A reversible or selectively cleavable linkage is a linkage that can be disrupted or cleaved without disrupting or cleaving other linkages in components of the system (such as in conjugated binding agents, solid support, etc.). Some examples of covalent binding agents can be found in US20040121405 A1 or in Du J, et al., cBinderDB: a covalent binding agent database. Bioinformatics. 2017 Apr. 15; 33(8):1258-1260. In another example, an engineered S—S bond can be established after binding of a peptide binding agent to macromolecule analyte, which can be reversed under reducing conditions.

In some embodiments, the first binding agent, the second binding agent and/or the higher order binding agent comprise(s) a polypeptide, such as an engineered polypeptide. In some embodiments, the first binding agent, the second binding agent and/or the higher order binding agent comprise(s) an aptamer. In some embodiments, the first binding agent, the second binding agent and/or the higher order binding agent comprise(s) a small molecule. In some embodiments, the first binding agent, the second binding agent and the higher order binding agent do not comprise a nucleic acid molecule. In some embodiments, the first binding agent, the second binding agent and the higher order binding agent each have a molecular weight of at least 1 kDa, at least 5 kDa, at least 10 kDa, at least 15 kDa, or at least 20 kDa.

In some embodiments, the RNAP portion A associated with the macromolecule analyte is releasably or reversibly attached to the solid support.

In some embodiments, the recording tag associated with the macromolecule analyte further comprises at least one transcription termination element.

In some embodiments, the recording tag associated with the macromolecule analyte further comprises at least one Poly(A) signal sequence.

In some embodiments, the disclosed methods are cell-free methods (e.g., all steps of the methods are performed outside a cell, such as in vitro).

In some embodiments, the disclosed methods further comprise attaching the macromolecule analyte to the recording tag attached to the solid support before performing step (a).

In some embodiments of the disclosed methods and systems, each binding agent of the plurality of binding agents comprises a polypeptide or an aptamer. In some embodiments, at step (b), the binding between the macromolecule analyte and the first binding agent is a non-covalent association. In other embodiments, binding agents that form covalent bonds with the macromolecule analyte are employed.

In some embodiments of the disclosed methods and systems, the recording tag associated with the macromolecule analyte consists of a single DNA molecule, which comprises the plurality of promoters. In other embodiments, the recording tag associated with the macromolecule analyte comprises a plurality of DNA molecules attached directly or indirectly to the solid support, and wherein each DNA molecule of the plurality of DNA molecules comprises a different promoter from the plurality of promoters (see, e.g., FIGS. 2-4).

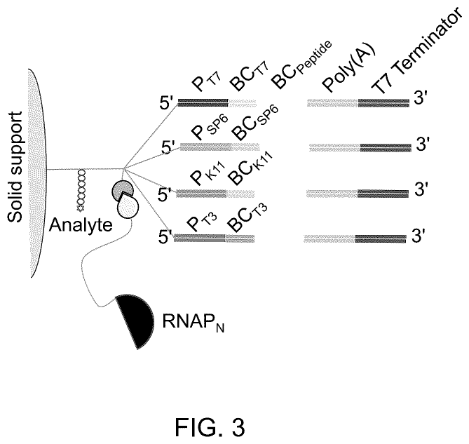

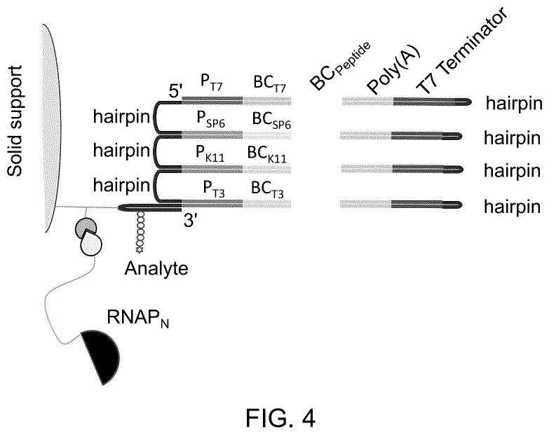

In some embodiments of the disclosed methods and systems, different recording tag architectures can be used. In one embodiment, four or more different DNA promoters are present on a single DNA molecule and conjugated to a single functional site on the solid support (i.e., bead), wherein one unique analyte-specific barcode (BCpepfide) (or multiple different analyte-specific barcodes) correspond to a single polypeptide covalently linked to the recording tag or a chemical linker fusing the bead to the recording tag (FIG. 1A and FIG. 2). In another embodiment, each of the different DNA promoters are present on a different DNA molecule, and these DNA molecules are conjugated to a single functional site on the solid support (i.e., bead) and each comprises an identical and unique analyte-specific barcode (BC_{Peptide}) (or multiple different analyte-specific barcodes) corresponding to the one polypeptide covalently linked to the recording tag (FIG. 3). In yet another embodiment, different DNA promoters are present on a single DNA molecule, which folds into a multi-hairpin structure that is conjugated to a single functional site on the solid support (i.e., bead), wherein one identical and unique analyte-specific barcode (BC_{Peptide}) (or multiple different analyte-specific barcodes) correspond to one polypeptide covalently linked to the recording tag (FIG. 4).

In preferred embodiments of the disclosed methods, RNA transcripts generated at each binding cycle are collected shortly after generation, and before repeating the contacting step (b) (contacting the macromolecule analyte with a plurality of binding agents). These RNA transcripts can be analyzed immediately, or stored until all binding cycles are completed for a given macromolecule analyte, followed by their analysis. Collecting RNA transcripts after each binding cycle greatly simplifies the analysis, since RNA transcript lengths and/or at least partial sequences can be used to identify the binding agent that was bound to the macromolecule analyte in a given binding cycle, which resulted in formation of the specific functional RNAP enzyme. When target binding specificity of binding agents is known, this information will result in knowledge of the identity of a specific portion or component of the macromolecule analyte to which the binding agent was bound in the current binding cycle. When RNA transcripts generated at each binding cycle are not collected efficiently, they may be present during the next binding cycle, complicating the analysis. In preferred embodiments of the disclosed methods, at least 50%, at least 60%, at least 70%, at least 80%, at least 90%, at least 95%, at least 99% of RNA transcripts generated at each binding cycle are collected and removed from the system to minimize their carry-over to the next binding cycle. In some embodiments, in each binding cycle, RNA transcript recovery rate is at least 50%, at least 60%, at least 70%, at least 80%, at least 90%, at least 95%, or at least 99%.

In some embodiments of the disclosed methods, to minimize or completely avoid cross-contamination (carry-over) of RNA transcripts between binding cycles, RNase enzyme is added to the system after RNA transcripts are collected. In one example, RNase enzyme can be added during the step of removing a portion of the macromolecule analyte (the cleavage step). Then, after completion of the removing step, RNase enzyme is washed away from the system before repeating steps of binding and generating RNA transcripts. In some embodiments, RNase inhibitors may be added during steps of binding and generating RNA transcripts to mitigate potential carry-over of RNase enzyme. In some embodiments, chemical degradation of RNA transcripts may be employed to avoid carry-over of RNA transcripts between binding cycles.

In some embodiments of the disclosed methods, at each binding cycle, the functional RNAP enzyme is disrupted after it initiated transcription from a cognate promoter of the plurality of promoters, and one or more copies of RNA transcripts are produced. In other embodiments, the functional RNAP enzyme is washed away from the recording tag associated with the macromolecule analyte. This can be done by disrupting interactions between the binding agent and the macromolecule analyte, as well as between the functional RNAP enzyme and its cognate promoter. In some embodiments, a washing buffer containing a detergent is applied to the system to disrupt the abovementioned interactions. Examples of detergents include, without limitation, 0.5-5% Tween 20, 0.5-5% NP-40 or NP-40 substitute, 0.5-2% CHAPS, 0.5-2% CHAPSO, 0.1-1% sarkosyl, and 0.1-0.5% sodium dodecyl sulfate. Alternatively, high salt buffers can be used for disruption of the abovementioned interactions. Efficient removal of the functional RNAP enzyme (together with removal of binding agents) from the system at each binding cycle increases specificity of the analysis, since carry-over of the functional RNAP enzyme to the next binding cycle may result in generation of erroneous signal, such as generation of RNA transcripts not related to interaction between the macromolecule analyte and the current binding agent(s). In preferred embodiments of the disclosed methods, at least 70%, at least 80%, at least 90%, at least 95%, at least 99% of the functional RNAP enzymes formed at each binding cycle (together with removal of binding agents) are removed (disrupted, inactivated, blocked from binding to the recording tag, and/or washed away from the recording tag) before repeating the steps of the next binding cycle.

Steps of collecting RNA transcripts generated at each binding cycle and disrupting the functional RNAP enzyme (or washing the functional RNAP enzyme away from the recording tag) can be performed in any order and independent of each other.

In preferred embodiments of the disclosed methods, the recording tag associated with the macromolecule analyte comprises an analyte-specific barcode. In some embodiments, the analyte-specific barcode is a nucleic acid and comprises 4, 5, 6, 7, 8, 9, 10, 15, 20, or more nucleotides. The analyte-specific barcode is particularly useful for high-throughput analysis of different macromolecule analytes, allowing multiplexing of analytes from different samples, such as biological samples.

In preferred embodiments of the disclosed methods and systems, the RNAP portion A and each of the RNAP portion Bs are fragments or modified fragments of a functional RNAP enzyme, and each of the RNAP portion Bs can form a functional RNAP enzyme together with the RNAP portion A when brought into sufficient proximity to interact with each other. Each of the functional RNAP enzymes is configured to initiate transcription from a specific promoter of the plurality of promoters associated with the analyzed macromolecule analyte. In some embodiments, the functional RNAP enzyme is an engineered version of an enzyme selected from the group consisting of T7 RNAP enzyme, T3 RNAP enzyme, K11 RNAP enzyme, and SP6 RNAP enzyme. In some embodiments, the functional RNAP enzyme comprises an amino acid sequence that is at least 30%, at least 35%, at least 40%, at least 45%, at least 50%, at least 60%, at least 70%, at least 75%, at least 80%, at least 85%, at least 90%, or at least 95% identical to any one of the amino acid sequences set forth in SEQ ID NO: 1-SEQ ID NO: 4.

In some embodiments, the RNAP portion A comprises an amino acid sequence that is at least 30%, at least 35%, at least 40%, at least 45%, at least 50%, at least 60%, at least 70%, at least 75%, at least 80%, at least 85%, at least 90%, or at least 95% identical to any one of the amino acid sequences set forth in SEQ ID NO: 5-SEQ ID NO: 9. In some embodiments, each of the RNAP portion Bs used in the system comprises an amino acid sequence that is at least 30%, at least 35%, at least 40%, at least 45%, at least 50%, at least 60%, at least 70%, at least 75%, at least 80%, at least 85%, at least 90%, or at least 95% identical to any one of the amino acid sequences set forth in SEQ ID NO: 10-SEQ ID NO: 43.

In preferred embodiments of the disclosed methods and systems, a set of orthogonal RNAP portion Bs is used, wherein each RNAP portion B from the set of the orthogonal RNAP portion Bs is: i) joined to a binding agent, and ii) is capable of forming a functional RNAP enzyme together with the RNAP portion A when brought into sufficient proximity to interact with each other, and wherein each of the functional RNAP enzymes formed is configured to initiate transcription from a different promoter of the plurality of promoters associated with the analyzed macromolecule analyte. Thus, different functional RNAP enzymes formed by the same RNAP portion A and any one of RNAP portion Bs from the set of the orthogonal RNAP portion Bs have orthogonal promoter specificity. Orthogonality can be achieved by engineering RNAP portion Bs to bind to different promoters and not cross-react. Several successful examples of orthogonal RNAP portion Bs are known in the art and disclosed in the following publications: US2020/0332371, US2015/0368625, US2020/0199599; Pu J, et al., 2017; Pu J, et al., J Am Chem Soc. 2017; Temme K, et al., 2012; Meyer A J, et al., 2015; Chelliserrykattil, et al., 2001 (see also Example 2, Example 3 and Example 4).

In one specific embodiment, the RNAP portion A has an amino acid sequence that is at least 80% identical to the amino acid sequence set forth in SEQ ID NO: 5, and a set of the RNAP portion Bs comprises four or more RNAP portion Bs, wherein each RNAP portion B comprises an amino acid sequence that is at least 80% identical to any one of the amino acid sequences set forth in SEQ ID NO: 10, SEQ ID NO: 14-SEQ ID NO: 20, and SEQ ID NO: 41-SEQ ID NO: 43.

In some embodiments, the RNAP portion A comprises 1, 2, 3, 4, 5, 6, or 7 amino acid substitutions that correspond to positions 32, 35, 63, 98, 107, 122, or 144 of SEQ ID NO: 5. In some specific embodiments, these amino acid substitutions are L32S, E35G, E63K, K98R, Q107K, T122S or A144T. In some specific embodiments, the RNAP portion A comprises sequence set forth in SEQ ID NO: 9. In some embodiments, the RNAP portion A comprises 1, 2, 3, 4, 5, 6, or 7 amino acid substitutions that correspond to positions 32, 35, 63, 98, 107, 122, or 144 of SEQ ID NO: 5, and has at least 80, 81, 82, 83, 84, 85, 86, 87, 88, 89, 90, 91, 92, 93, 94, 95, 96, 97, 98, or 99% sequence identity to amino acid sequence set forth in SEQ ID NO: 5.

In some embodiments, the RNAP portion A is engineered to achieve a proximity-dependent interaction with each of the RNAP portion Bs.

In another specific embodiment, the RNAP portion A has an amino acid sequence that is at least 80% identical to the amino acid sequence set forth in SEQ ID NO: 9, and a set of the RNAP portion Bs comprises four or more RNAP portion Bs, wherein each RNAP portion B comprises an amino acid sequence that is at least 80% identical to any one of the amino acid sequences set forth in SEQ ID NO: 10, SEQ ID NO: 14-SEQ ID NO: 20, and SEQ ID NO: 41-SEQ ID NO: 43.

In some embodiments, the plurality of promoters present in the recording tag associated with the macromolecule analyte comprises any one of the nucleotide sequences set forth in SEQ ID NO: 44-SEQ ID NO: 51. In some particular embodiments, the plurality of promoters present in the recording tag associated with the macromolecule analyte comprises two, three, four, five or more of the nucleotide sequences set forth in SEQ ID NO: 44-SEQ ID NO: 51. In some additional embodiments, each of the promoters set forth in the SEQ ID NO: 44-SEQ ID NO: 51 when present in the plurality of promoters further comprises a stabilization sequence GGGAGA located at 3′ terminus of each of the promoters, as disclosed in Temme K, et al., 2012.

In some embodiments, each binding agent of the plurality of binding agents comprising an RNAP portion B does not comprise a polynucleotide and consists essentially of polypeptide molecule(s) optionally joined by a chemical linker. The advantage of these embodiments is avoiding potentially unpredictable nucleic acid-nucleic acid interactions of the binding agent with the immobilized recording tags, which may partially compromise assay specificity and/or reproducibility.

In some embodiments, background interactions between the RNAP portion A and each of the RNAP portion Bs are reduced by introducing mutations to interaction surface(s) of the RNAP fragments (as disclosed in US2020/0332371 A1 and Pu et al., Nat Chem Biol, 2017).

In some embodiments, the RNAP portion A and each of the RNAP portion Bs of the plurality of binding agents are enzymatically inactive (e.g., do not initiate transcription of DNA to generate RNA transcripts) when not associated with each other.

In some embodiments, the RNAP portion A and each of the RNAP portion Bs of the plurality of binding agents have no RNA polymerase activity by themselves.

In some embodiments, the RNAP portion A has no ability to bind to a promoter of the plurality of promoters; and wherein each of the RNAP portion Bs of the plurality of binding agents has specificity for a promoter of the plurality of promoters, but does not initiate transcription of RNA without binding to the RNAP portion A.

In some embodiments, the interaction between the RNAP portion A and the RNAP portion B to form the first functional RNAP enzyme is increased relative to the interaction between the RNAP portion A and the RNAP portion B without joining to the first binding agent through the binding agent-macromolecule analyte interaction.

In some embodiments, a method for performing at least partial sequence identification of a polypeptide analyte is disclosed, comprising:
(a) providing the polypeptide analyte attached to a solid support, wherein the polypeptide analyte is associated with (i) an RNAP portion A, and (ii) a recording tag comprising a plurality of promoters, and optionally comprising an analyte-specific barcode;
(b) modifying an N-terminal amino acid (NTAA) residue of the polypeptide analyte with a chemical moiety to generate a functionalized NTAA residue, and contacting the polypeptide analyte with a plurality of binding agents capable of binding to functionalized NTAA residues of polypeptides, wherein each binding agent of the plurality of binding agents is joined to an RNAP portion B, whereby binding between the functionalized NTAA residue of the polypeptide analyte and a first binding agent of the plurality of binding agents brings the RNAP portion A and an RNAP portion B joined to the first binding agent into sufficient proximity to interact with each other and form a first functional RNAP enzyme configured to initiate transcription from a first promoter of the plurality of promoters;
(c) following binding of the first binding agent to the functionalized NTAA residue, generating a first RNA transcript by the first functional RNAP enzyme from the recording tag associated with the polypeptide analyte;
(d) collecting the first RNA transcript, and disrupting the first functional RNAP enzyme or washing the first functional RNAP enzyme away from the recording tag associated with the polypeptide analyte;
(e) removing the functionalized NTAA residue of the polypeptide analyte to generate a newly exposed NTAA of the polypeptide analyte;
(f) repeating steps (b), (c), (d) and (e) sequentially one or more times by replacing the NTAA residue with a newly exposed NTAA residue of the polypeptide analyte, and the first binding agent with a second or higher order binding agent capable of binding to newly functionalized NTAA residue(s) of the polypeptide analyte, wherein binding between the newly functionalized NTAA residue(s) of the polypeptide analyte and a second or higher order binding agent of the plurality of binding agents brings the RNAP portion A and an RNAP portion B joined to the second or higher order binding agent into sufficient proximity to interact with each other and form a second or higher order functional RNAP enzyme configured to initiate transcription from a second or higher order promoter of the plurality of promoters, thereby generating and collecting a second and, optionally, higher order RNA transcript;
(g) analyzing the first, the second and, optionally, the higher order RNA transcripts, which comprises identifying RNA transcript lengths and/or at least partial sequences of the first, the second and, optionally, the higher order RNA transcripts, thereby identifying the NTAA and one or more newly exposed NTAA residue(s) of the polypeptide analyte.

In some embodiments, the method further includes providing the plurality of polypeptides with an RNAP portion A. For example, if a sample is obtained, the sample is treated and processed to provide the polypeptides with an RNAP portion A. An attachment step may be performed to join a nucleic acid recording tag comprising a plurality of promoters or the RNAP portion A to the polypeptides. In some cases, each polypeptide or a majority of polypeptides are provided and associated with an RNAP portion A and a nucleic acid recording tag. In some embodiments, the plurality of polypeptides is provided with an RNAP portion A during, prior to, or after providing the polypeptide and the associated nucleic acid recording tag joined to a support. In some particular embodiments, the polypeptides are immobilized to the support after providing the polypeptides with the nucleic acid recording tag.

In multiple embodiments of the disclosed methods and systems, macromolecule analyte(s), a nucleic acid recording tag comprising a plurality of promoters, and an RNAP portion A can be joined to the support, directly or indirectly, by any means known in the art, including covalent and non-covalent interactions, or in any combination thereof. In some embodiments, macromolecule analyte, an associated RNAP portion A and an associated nucleic acid recording tag may be each independently joined to a solid support in a proximity to each other, so they are associated with each other indirectly, via the support; alternatively, a nucleic acid recording tag can be joined to a support, and the macromolecule analyte can be joined to the recording tag by direct joining or by first attaching a short polynucleotide to the macromolecule analyte, followed by nucleic acid hybridization between the short polynucleotide and the immobilized recording tag; alternatively, the RNAP portion A and the macromolecule analyte can be joined to a support via a linker, wherein the linker is a tri-functional linker that comprises: a moiety for attachment to the polypeptide, a moiety for attachment to the support, and a moiety for attachment to the RNAP portion A. In some embodiments, the RNAP portion A is directly or indirectly joined to the polypeptide. In some embodiments, the support can include an agent or coating to facilitate joining, either direct or indirectly, the macromolecule analyte, the RNAP portion A, or the recording tag, to the support. Any suitable molecule or materials may be employed for this purpose, including proteins, nucleic acids, carbohydrates and small molecules. In some embodiments, the polypeptide, and/or RNAP portion A, and/or recording tag, may be joined to the solid support or each other via ligation. In other embodiments, the polypeptide, and/or RNAP portion A, and/or recording tag, may be joined to the solid support or each other via affinity binding pairs (e.g., biotin and streptavidin). In some particular embodiments, the polypeptide is not directly connected to the RNAP portion A, but the two are in sufficient proximity of each other. The distance between the polypeptide and associated RNAP portion A may be adjusted based on the length of the linker and/or the distance between the binding agent and the RNAP portion B. Multiple other types of immobilization and association between a macromolecule analyte, an associated RNAP portion A and an associated nucleic acid recording tag are possible. Various configurations can be used for joining the polypeptides and the RNAP portion As associated or co-localized directly or indirectly with the polypeptide, to the support. In preferred embodiments, the described immobilization (attachment to the support) of these three components is configured so that after binding reaction, the RNAP portion A and the RNAP portion B can interact with each other and generate a functional RNAP enzyme configured to initiate transcription from a promoter of the plurality of promoters of the recording tag.

In some embodiments, the RNAP portion A is covalently conjugated to the solid support via a structured polypeptide linker capable of unfolding and refolding during the RNA polymerization reaction. For example, the structured polypeptide linker may be a helical bundle such as a designed helical repeat proteins (DHRs) (disclosed in Brunette T J et al., 2020) or monomeric coiled-coil proteins (see Boyken S E, et al., 2016 and Boyken S E, et al., 2019). In these embodiments, the structured polypeptide linker may be configured to uncoil and unfold during nucleotide triphosphate (NTP) hydrolysis by the reconstituted RNAP enzyme before RNA polymerization, permitting the covalently conjugated RNAP portion A to extend toward the 3′ terminus of the DNA recording tag conjugated to the solid support. Upon termination of the RNA polymerization at the 3′ terminus of the DNA recording tag, lack of NTP hydrolysis permits re-coiling and re-folding of the structured polypeptide linker, effectively restoring the reaction site to a state poised for binding agent-fused RNAP portion B binding to the polypeptide analyte and subsequent RNA polymerization again for the higher order RNA transcript production.

In another embodiment, flexible peptide linker or chemical linker that connects the RNAP portion A to the solid support is inducibly releasable, optionally by an inducible method.

In yet another embodiment, in order to decrease the background RNA polymerization in the absence of the RNAP portion B (RNAPC)-fused binding agent contacting the macromolecule analyte, the affinity of the RNAPN-RNAPC fragment interaction is decreased by mutations at the RNAPN-RNAPC interaction interface. The interface may be weakened via state-of-the-art computational protein design methodologies via selecting residues at the RNAPN-RNAPC interface and making amino acid substitutions that decrease the magnitude of the predicted binding energy of the RNAPN-RNAPC interaction. In some embodiments, for T7 RNAPN and T7 RNAPC fragments, the mutated residues are L32, E35, E63, K98, Q107, T122 and/or A144 corresponding to SEQ ID NO: 5. In one particular embodiment, the mutated residues in the RNAP portion A are L32S, E35G, E63K, K98R, Q107K, T122S and/or A144T corresponding to SEQ ID NO: 5 (SEQ ID NO: 9).

In some embodiments, a linker is used to join the RNAP portion A to the support, the macromolecule analyte to the support, the recording tag to the support, the recording tag to the polypeptide, or any combination thereof. Such linkers may join the polypeptide and the RNAP portion A to the solid support by covalent or non-covalent interactions, or a mixture thereof, and the linker may comprise multiple components. Such linkers are optional and direct attachment between the various components is within the scope of this disclosure. In some embodiments, the linker is a moiety for associating with the polypeptide and a moiety for associating with the RNAP portion A. For example, the joining uses a linker which comprises an azide group, which can react with an alkynyl group in another molecule to facilitate association or binding between the solid support and the other molecule. In some embodiments, the linker comprises a biotin. In some embodiments, the linker comprises alkyl, PEG, or PEO moiety with 2-18 chain lengths. In some cases, the RNAP portion A is configured to bind the biotin. In some embodiments, the RNAP portion A is associated with a hapten-binding group. For example, the hapten-binding group is streptavidin. In some examples, the hapten-binding group and the RNAP portion A are chemically or genetically attached. In some examples, the chemical attachment is a covalent attachment via a linker molecule.

In some embodiments, the linker is a tri-functional linker. For example, the tri-functional linker may include a moiety to associating with the polypeptide; a moiety for associating with the support; and a moiety for associating with the recording tag (or RNAP portion A). A linker can be any molecule (e.g., protein, nucleic acid, carbohydrate, small molecule, etc.) capable of associating or binding a polypeptide to a solid support.

In one embodiment, the linker used to join the polypeptide and the RNAP portion A to the solid support has the following structure (L-1):

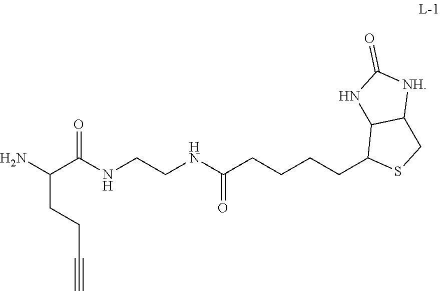 [SMILES: C#CCCC(N)C(=O)NCCNC(=O)CCCCC1SCC2NC(=O)NC21]

Linker L-1 contains an amine group which can bind the polypeptide by, for example, formation of an amide bond with the carboxylate of tryptic peptides using 1-ethyl-3-(3-dimethylaminopropyl) carbodiimide (EDC) coupling. Further, the alkynyl group provides for joining of L-1 to a solid support bearing an azide group through click chemistry. Lastly, L-1 also contains a biotin moiety, which can be bound by streptavidin linked to the RNAP portion A. As illustrated in this embodiment, L-1 serves to join both the peptide and the RNAP portion A to the solid support by both covalent (amide bond formation and click chemistry) and non-covalent binding (biotin-streptavidin interaction), both of which are encompassed within the practice of this disclosure.

The linker can have the following structure:

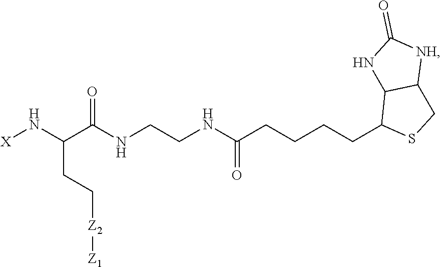

wherein:
   X is the polypeptide; and
   Z₁-Z₂ is C≡C and is capable of binding to the solid support.

The linker can be trifunctional, as it can (1) associate or bind to a solid support; (2) associate or bind to a polypeptide to be analyzed or sequenced (3) associate or bind to a hapten-binding protein (when the RNAP portion A comprises a hapten molecule). The association or binding can be covalent or non-covalent.

In certain embodiments, the linker joins two or three molecules via enzymatic reaction or chemistry reaction (e.g., a click chemistry reaction). In some embodiments, macromolecule analytes can first be derivatized with reactive groups such as click chemistry moieties. The activated macromolecule analytes can then be attached to a suitable solid support and then labeled with recording tags using the complementary click chemistry moiety. As an example, macromolecule analytes derivatized with alkyne and mTet moieties may be immobilized to beads derivatized with azide and TCO and attached to recording tags labeled with azide and TCO. It is understood that the methods provided herein for attaching macromolecule analytes to the solid support may also be used to attach recording tags to the solid support, attach RNAP portion A to the solid support, or attach recording tags to macromolecule analytes.

Exemplary click chemistry reactions useful to generate attachments of the disclosed components of the system to a solid support or to each other include, without limitation, the copper catalyzed reaction of an azide and alkyne to form a triazole (Huisgen 1, 3-dipolar cycloaddition), strain-promoted azide alkyne cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse electron demand Diels-Alder (IEDDA) reaction (e.g., m-tetrazine (mTet) or phenyl tetrazine (pTet) and trans-cyclooctene (TCO)); or pTet and an alkene), alkene and tetrazole photoreaction, Staudinger ligation of azides and phosphines, and various displacement reactions, such as displacement of a leaving group by nucleophilic attack on an electrophilic atom (Horisawa 2014, Knall, Hollauf et al. 2014). Exemplary displacement reactions include reaction of an amine with: an activated ester; an N-hydroxysuccinimide ester; an isocyanate; an isothioscyanate, an aldehyde, an epoxide, or the like. In some embodiments, iEDDA click chemistry is used for immobilizing polypeptides to a support since it is rapid and delivers high yields at low input concentrations. In another embodiment, m-tetrazine rather than tetrazine is used in an iEDDA click chemistry reaction, as m-tetrazine has improved bond stability. In another embodiment, phenyl tetrazine (pTet) is used in an iEDDA click chemistry reaction. In one case, a polypeptide is labeled with a bifunctional click chemistry reagent, such as alkyne-NHS ester (acetylene-PEG-NHS ester) reagent or alkyne-benzophenone to generate an alkyne-labeled polypeptide. In some embodiments, an alkyne can also be a strained alkyne, such as cyclooctynes including Dibenzocyclooctyl (DBCO), etc.

The linker may comprise an amine group, which can form an amide bond with the carboxylate of tryptic peptides via 1-Ethyl-3-(3-dimethylaminopropyl) carbodiimide (EDC) coupling. The alkyne group of the trifunctional reagent allows the association or binding of macromolecules, such as polypeptides, to a solid support bead coated with an azide group through bio-orthogonal click chemistry.

In some embodiments, the linker can be prepared by following a solid phase synthesis. For example, Biotin NovaTag resin (Millipore) is deprotected with 20% piperidine to remove the Fmoc group, it is then coupled with N-Fmoc-L-propargylglycine (Sigma) in the presence of HBTU (Sigma). After the Fmoc group is removed by 20% piperidine, the reagent is cleaved from the beads by 95% TFA and purified by HPLC.

In some embodiments, tri-functional linker is an amino acid-based linker, such as lysine-based tri-functional linker. Amino acids provide a unique molecular scaffold to derive “trifunctional” linkers through separate modification of the N-terminus, C-terminus, and sidechain (natural or unnatural). For example, amino acid side chains, may be functionalized with various attachment tags using standard amine modification chemistry or produced with a pre-installed attachment tag (e.g., biotin, desthiobiotin, mTET, photoreactive tags (diazirine, benzophenone, etc.)). C-terminal carboxylates can be converted into reactive esters through standard chemistries (CDI, EDC, etc.), provided the N-terminus is protected to prevent polymerization of the reagent.

The solid support can further include an agent (e.g., reacting agent) or coating to facilitate the direct or indirect binding of a macromolecule, such as polypeptide, or other component of the instant system, to the solid support. The reacting agent can be any molecule (e.g., protein, nucleic acid, carbohydrate, small molecule). The reacting agent can be an affinity molecule. The reacting agent can be an azide group. In embodiments where the reacting agent is an azide group, the azide group can react with an alkyne group in another molecule to facilitate association or binding between the solid support and the other molecule.

In some embodiments, the methods provided herein include forming a complex which can comprise a support, a linker (e.g., linking an RNAP portion A) and a macromolecule analyte. For example, the complex can be formed by reacting the RNAP portion A with a solid support to form a linker-solid support complex, and then reacting the linker-solid support complex with the macromolecule analyte joined to an associated recording tag. The complex can also be formed by reacting the linker with the macromolecule analyte to form a linker-macromolecule complex, and then reacting the linker-macromolecule complex with the solid support. The association or binding between the linker, support and macromolecule can be covalent or non-covalent. In some embodiments, the complex can be formed by reaction of an amine group in the first molecule and a carboxyl group in the polypeptide analyte. The first complex can be formed via a 1-ethyl-3-(3-dimethylaminopropyl)carbodiimide (EDC) coupling reaction.

Provided herein are methods for assaying a macromolecule, such as a peptide. The methods of the present invention also permit the detection, quantitation or analysis of a plurality of macromolecules, such as peptides (two or more peptides) simultaneously, e.g., multiplexing. Simultaneously as used herein refers to detection, quantitation or sequencing of a plurality of peptides in the same assay. The plurality of macromolecules detected, quantitated and/or analyzed can be present in the same sample, e.g., biological sample, or different samples. The plurality of macromolecules can be derived from the same subject or different subjects. In some embodiments, the method is performed on a plurality of isolated macromolecules from a sample. In some embodiments, the macromolecules are of unknown identity. The plurality of macromolecules, such as polypeptides, that are analyzed can be different polypeptides, or the same polypeptide derived from different samples. A plurality of macromolecule analytes includes 2 or more macromolecules, 5 or more macromolecules, 10 or more macromolecules, 50 or more macromolecules, 100 or more macromolecules, 500 or more macromolecules, 1000 or more macromolecules, 5,000 or more macromolecules, 10,000 or more macromolecules, 50,000 or more macromolecules, 100,000 or more macromolecules, 500,000 or more macromolecules, or 1,000,000 or more macromolecules.

Macromolecules for analysis (macromolecule analytes) using the provided methods may be obtained from a source and treated in various ways. In some cases, the provided methods are useful on macromolecules obtained from a sample and are of unknown identity. In some cases, the macromolecules are obtained from a mixture of macromolecules from a sample. In some embodiments, the proteins, polypeptides, or peptides are obtained from a sample that is a biological sample. In some embodiments, the sample comprises but is not limited to, mammalian or human cells, yeast cells, and/or bacterial cells. In some embodiments, the sample contains cells that are from a sample obtained from a multicellular organism. For example, the sample may be isolated from an individual. In some embodiments, the sample may comprise a single cell type or multiple cell types. In some embodiments, the sample may be obtained from a mammalian organism or a human, for example by puncture, or other collecting or sampling procedures. In some embodiments, samples may be derived from non-biological origin such as from an automated peptide synthesizer.

A polypeptide analyte may comprise L-amino acids, D-amino acids, or both. A polypeptide analyte or protein complex to be analyzed may comprise a standard, naturally occurring amino acid, a modified amino acid (e.g., post-translational modification), an amino acid analog, an amino acid mimetic, or any combination thereof. In some embodiments, a polypeptide analyte is naturally occurring, synthetically produced, or recombinantly expressed. In any of the aforementioned polypeptide analyte embodiments, a polypeptide analyte may further comprise a post-translational modification.

In certain embodiments, a polypeptide or protein can be fragmented before analyzing by the provided methods. Polypeptides or proteins can be fragmented by any means known in the art, including fragmentation by a protease or endopeptidase. In some embodiments, fragmentation of a peptide, polypeptide, or protein is targeted by use of a specific protease or endopeptidase. A specific protease or endopeptidase binds and cleaves at a specific consensus sequence (e.g., TEV protease). In other embodiments, fragmentation of a peptide, polypeptide, or protein is non-targeted or random by use of a non-specific protease or endopeptidase. Chemical reagents can also be used to digest proteins into peptide fragments. A chemical reagent may cleave at a specific amino acid residue (e.g., cyanogen bromide hydrolyzes peptide bonds at the C-terminus of methionine residues). Chemical reagents for fragmenting polypeptides or proteins into smaller peptides include cyanogen bromide (CNBr), hydroxylamine, hydrazine, formic acid, BNPS-skatole [2-(2-nitrophenylsulfenyl)-3-methylindole], iodosobenzoic acid, NTCB+Ni (2-nitro-5-thiocyanobenzoic acid), etc.

In certain embodiments, following enzymatic or chemical cleavage, the resulting polypeptide analytes have approximately similar desired length, e.g., from about 4 amino acids to about 70 amino acids. A cleavage reaction may be monitored, preferably in real time, by spiking the protein or polypeptide sample with a short test peptide capable of FRET (fluorescence resonance energy transfer) that comprises a peptide sequence containing a proteinase or endopeptidase cleavage site. In the intact FRET peptide, a fluorescent group and a quencher group are attached to either end of the peptide sequence containing the cleavage site, and FRET between the quencher (acceptor) and the fluorophore (donor) leads to low donor fluorescence intensity. Upon cleavage of the test peptide by a protease or endopeptidase, the quencher and fluorophore are separated giving a large increase in donor fluorescence intensity. A cleavage reaction can be stopped when a certain fluorescence intensity is achieved, allowing a reproducible cleavage endpoint to be achieved.

In some embodiments, the sample can undergo protein fractionation methods where the protein or polypeptide analytes are separated by one or more properties such as cellular location, molecular weight, hydrophobicity, isoelectric point, or protein enrichment methods. In some embodiments, a subset of macromolecules (e.g., proteins) within a sample is fractionated such that a subset of the macromolecules is sorted from the rest of the sample. For example, the sample may undergo fractionation methods prior to attachment to a support. Alternatively, or additionally, protein enrichment methods may be used to select for a specific protein or polypeptide (see, e.g., Whiteaker et al., 2007, Anal. Biochem. 362:44-54, incorporated by reference in its entirety) or to select for a particular post translational modification (see, e.g., Huang et al., 2014. J. Chromatogr. A 1372:1-17, incorporated by reference in its entirety). Alternatively, a particular class or classes of proteins such as immunoglobulins, or immunoglobulin (Ig) isotypes such as IgG, can be affinity enriched or selected for analysis. Overly abundant proteins can also be subtracted from the sample using standard immunoaffinity methods. Depletion of abundant proteins can be useful for plasma samples where over 80% of the protein constituent is albumin and immunoglobulins. Several commercial products are available for depletion of plasma samples of overly abundant proteins, including depletion spin columns that remove top 2-20 plasma proteins (Pierce, Agilent), or PROTIA and PROT20 (Sigma-Aldrich).

In certain embodiments, a protein sample dynamic range can be modulated by fractionating the protein sample using standard fractionation methods, including electrophoresis and liquid chromatography (Zhou et al., 2012, Anal Chem 84(2): 720-734), or partitioning the fractions into compartments (e.g., droplets) loaded with limited capacity protein binding beads/resin (e.g., hydroxylated silica particles) (McCormick, 1989, Anal Biochem 181(1): 66-74) and eluting bound protein. Excess protein in each compartmentalized fraction is washed away. Examples of electrophoretic methods include capillary electrophoresis (CE), capillary isoelectric focusing (CIEF), capillary isotachophoresis (CITP), free flow electrophoresis, gel-eluted liquid fraction entrapment electrophoresis (GELFrEE). Examples of liquid chromatography protein separation methods include reverse phase (RP), ion exchange (IE), size exclusion (SE), hydrophilic interaction, etc. Examples of compartment partitions include emulsions, droplets, microwells, physically separated regions on a flat substrate, etc. Exemplary protein binding beads/resins include silica nanoparticles derivatized with phenol groups or hydroxyl groups (e.g., StrataClean Resin from Agilent Technologies, RapidClean from LabTech, etc.). By limiting the binding capacity of the beads/resin, highly-abundant proteins eluting in a given fraction will only be partially bound to the beads, and excess proteins removed.

A polypeptide analyzed in accordance with this disclosure may be enriched prior to analysis. Methods for enriching polypeptides of interest can include removing and collecting the polypeptides of interest from a sample (direct enrichment) or removing or subtracting other polypeptides from the sample (indirect enrichment), or both. Enrichment can increase the efficiency of the disclosed methods, improve dynamic range and improve the ability to detect many low abundance proteins in a complex sample. The methods of enrichment can include, but are not limited to, removing abundant species, such as albumin; enrich/subtract specific targeting of particular proteins (e.g., by antibody capture); enrich/subtract by general properties of proteins (e.g., size, isoelectic point (pI), hydrophobicity, etc.); enrich/subtract by targeting classes of proteins (e.g., by modification, such as phosphorylated proteins and glycosylated proteins); by ability to bind certain molecules (e.g., DNA binding proteins); enrich/subtract by subcellular localization (e.g., nuclear, mitochondrial, Golgi/ER, etc.); enrich/subtract by cellular population (e.g., T-cells, B-cells, etc.) that can be identified & sorted or otherwise captured (e.g., via cell surface markers). Methods and techniques for enrichment include, but are not limited to, centrifugation, chromatography, electrophoresis, binding, filtration, precipitation and degradation.

In some embodiments, a sample of peptides, polypeptides, or proteins can be processed into a physical area or volume e.g., into a compartment. Various processing and/or labeling steps may be performed on the sample prior to performing the binding reaction. In some embodiments, the compartment separates or isolates a subset of macromolecules from a sample of macromolecules. In some examples, the compartment may be an aqueous compartment (e.g., microfluidic droplet), a solid compartment (e.g., picotiter well or microtiter well on a plate, tube, vial, bead), or a separated region on a surface. In some cases, a compartment may comprise one or more beads to which macromolecules may be immobilized. In some embodiments, macromolecules in a compartment are labeled with a barcode. For example, the macromolecules in one compartment can be labeled with the same barcode or macromolecules in multiple compartments can be labeled with the same barcode. See e.g., Valihrach et al., Int J Mol Sci. 2018 Mar. 11; 19(3). pii: E807.

In certain embodiments where multiple polypeptides are immobilized on the same support, the polypeptides can be spaced appropriately to accommodate methods of performing the binding reaction and any downstream detection and/or analysis steps to be used to assess the polypeptide. For example, it may be advantageous to space the molecules optimally to ensure specificity of the signal detection step. In some embodiments, the polypeptides are immobilized on a support and spaced at optically resolvable distances.

To control spacing of the reaction sites (or immobilized macromolecule analytes, recording tags, or RNAP portion As) on the support (see FIG. 1A), the density of functional coupling groups for attaching the recording tag, polypeptide (e.g., TCO or carboxyl groups (COOH)) and/or the RNAP portion A may be titrated on the substrate surface. In some embodiments, multiple molecules are spaced apart on the surface or within the volume (e.g., porous supports) of a support such that adjacent molecules are spaced apart at a distance of about 50 nm or more, about 50 nm to about 500 nm, or about 50 nm to about 100 nm. In some embodiments, multiple molecules are spaced apart on the surface of a support with an average distance of at least 50 nm. In some embodiments, appropriate spacing of the macromolecule analytes, recording tags and/or RNAP portion As on the support is accomplished by titrating the ratio of available attachment molecules on the substrate surface. In some examples, the substrate surface (e.g., bead surface) is functionalized with a carboxyl group (COOH) which is treated with an activating agent (e.g., activating agent is EDC and Sulfo-NHS). In some examples, the substrate surface (e.g., bead surface) comprises NHS moieties. In some embodiments, a mixture of mPEGₙ-NH₂ and NH₂-PEG-mTet is added to the activated beads (wherein n is any number, such as 1-100). The ratio between the mPEG₃-NH₂ (not available for coupling) and NH₂-PEG₂₄-mTet (available for coupling) is titrated to generate an appropriate density of functional moieties available to attach the polypeptides on the substrate surface. In certain embodiments, the mean spacing between coupling moieties (e.g., NH₂-PEG₄-mTet) on the solid surface is at least 50 nm, at least 100 nm, at least 250 nm, or at least 500 nm. In some embodiments, the spacing of the polypeptides on the support is achieved by controlling the concentration and/or number of available COOH or other functional groups on the support.

In some embodiments, the RNAP portion B is directly or indirectly joined to the binding agent. In some embodiments, a binding agent is joined to an RNAP portion B via SpyCatcher-SpyTag interaction. The SpyTag peptide forms an irreversible covalent bond to the SpyCatcher protein via a spontaneous isopeptide linkage, thereby offering a genetically encoded way to create peptide interactions that resist force and harsh conditions (Zakeri et al., 2012, Proc. Natl. Acad. Sci. 109:E690-697; Li et al., 2014, J. Mol. Biol. 426:309-317). A binding agent may be expressed as a fusion protein comprising the SpyCatcher protein. In other embodiments, a binding agent is joined to an RNAP portion B via SnoopTag-SnoopCatcher peptide-protein interaction. The SnoopTag peptide forms an isopeptide bond with the SnoopCatcher protein (Veggiani et al., Proc. Natl. Acad. Sci. USA, 2016, 113:1202-1207). A binding agent may be expressed as a fusion protein comprising the SnoopCatcher protein. In yet other embodiments, a binding agent is joined to an RNAP portion B via the HaloTag protein fusion tag and its chemical ligand (or similar protein fusion tag, such as SNAP-tag, or CLIP-tag). HaloTag is a modified haloalkane dehalogenase designed to covalently bind to synthetic ligands (HaloTag ligands) (Los et al., 2008, ACS Chem. Biol. 3:373-382). The synthetic ligands comprise a chloroalkane linker attached to a variety of useful molecules. A covalent bond forms between the HaloTag and the chloroalkane linker that is highly specific, occurs rapidly under physiological conditions, and is essentially irreversible. In some embodiments, a binding agent is joined to an RNAP portion B using a cysteine bioconjugation method. In some embodiments, a binding agent is joined to an RNAP portion B using 7r-clamp-mediated cysteine bioconjugation (See e.g., Zhang et al., Nat Chem. (2016) 8(2):120-128). In some cases, a binding agent is joined to an RNAP portion B using 3-arylpropiolonitriles (APN)-mediated tagging (e.g., Koniev et al., Bioconjug Chem. 2014; 25(2):202-206). In preferred embodiments, the RNAP portion Bs and binding agents are protein-based molecules that may be genetically encoded and expressed as a single molecule connected by, but not limited to, a flexible amino acid linker (i.e., lacking secondary structure), a rigid amino acid linker (i.e., harboring secondary structure), or a folded polypeptide (harboring secondary structure and tertiary structure) such as hyperstable constrained peptides (Bhardwaj (3, et al. Accurate de novo design of hyperstable constrained peptides. Nature. 2016; 538(7625):329-335).

The methods described herein use a binding agent capable of binding to macromolecule analytes. The binding reaction may be performed by contacting a plurality of binding agents with a single polypeptide, or contacting a plurality of binding agents to a plurality of polypeptides, wherein the binding agents are provided sequentially or simultaneously. In preferred embodiments, each binding agent is associated with an RNAP portion B which is capable of generating a different detectable signal upon binding of the binding agent to the polypeptide analyte. In some embodiments, each of the RNAP portion Bs of the plurality of binding agents, when brought into sufficient proximity with the RNAP portion A, form a different functional RNAP enzyme that generates a unique RNA transcript while initiating transcription from a cognate promoter of the plurality of promoters. The unique RNA transcript is dependent on the identity of the target of the binding agent.

In some embodiments, binding agents each conjugated to a RNAP portion B are immunoglobulin domains or fragments thereof recognizing particular epitopes in macromolecule analytes. Based on obtained binding history of macromolecule analytes, the presence of particular epitopes in macromolecule analytes can be predicted. In some examples, the binding agent comprises an antibody, an antigen-binding antibody fragment, a single-domain antibody (sdAb), a Fv, a linear antibody, a diabody, an aptamer, a peptide mimetic molecule, a fusion protein, a reactive or non-reactive small molecule, or a synthetic molecule such as a de novo designed miniprotein inhibitor (Cao L, et al. De novo design of picomolar SARS-CoV-2 miniprotein inhibitors. Science. 2020; 370(65153:426-43 I) or a de novo designed immunoglobulin-like domain (Chidyausiku T M, et at. De novo design of immunoglobulin-like domains. Nat Commun. 2022; 13(1):5661). In other embodiments, binding agents may be genetically-encoded native proteins; e.g., a pathogenic protein such as, but not limited to, botulinum toxin, α-cobratoxin, or SARS-CoV-2 Receptor Binding Domain (RBD). Such a system allows, but is not limited to, immobilizing Ig fragments from human serum samples onto a solid support and detecting the presence of neutralizing antibodies by binding to a pathogenic protein joined to RNAP portion B, resulting in the generation RNA transcript outputs.

In some embodiments, binding agents are used that have binding affinity to specific functionalized NTAA residues. In some embodiments, engineered binding agents specific for functionalized NTAA residues and use thereof in the methods disclosed herein can be derived from natural lipocalin scaffolds, as disclosed in US 2023/0220589 A1. In these embodiments, an engineered binding agent that specifically binds to an N-terminally modified target polypeptide modified by an N-terminal modifier agent is used, wherein:
(i) the N-terminally modified target polypeptide has a formula: M-P1-P2-polypeptide, wherein M is an N-terminal modification, P1-P2-polypeptide is a target polypeptide before modification with the N-terminal modifier agent, M-P1 is a modified N-terminal amino acid (NTAA) residue of the target polypeptide, and P2 is a penultimate terminal amino acid residue of the target polypeptide;
(ii) the engineered binding agent specifically binds to the N-terminally modified target polypeptide through interaction between the engineered binding agent and the M-P1(or M-P1-P2) of the N-terminally modified target polypeptide; and
(iii) the engineered binding agent comprises an amino acid sequence having at least about 80% , at least about 90%, or at least about 95% sequence identity to the amino acid sequence selected from the group consisting of SEQ ID NO: 52 and SEQ ID NO: 53. In some other preferred embodiments, the engineered binding agent comprises an amino acid sequence having at least about 80% or 90% sequence identity to the amino acid sequence selected from the group consisting of SEQ ID NO: 52 and SEQ ID NO: 53, or other engineered binding agent specific to particular functionalized NTAA residues, as disclosed in US 2023/0220589 A1.

In preferred embodiments, a set or a plurality of engineered binding agents specific for various functionalized NTAA residues and each conjugated to an RNAP portion B is used in the methods disclosed herein. Such binding agents may have different affinities towards different functionalized NTAA residues. In some preferred embodiments, a set of engineered binding agents specific for functionalized NTAA residues comprises at least 3, 4, 5, 6, 7, 8, 9, 10, 15, or 20 engineered binding agents, wherein engineered binding agents from the plurality of engineered binding agents are configured to bind specifically (i.e., with specificity) to different functionalized NTAA residues of target polypeptides modified with the same or different N-terminal functionalizing reagents.

In some embodiments, engineered binding agents specific for functionalized NTAA residues and use thereof in the methods disclosed herein can be derived from natural metalloprotein scaffolds, as disclosed in US patent publication US 2022/0283175 A1. In these embodiments, an engineered metalloprotein binding agent that specifically binds to an N-terminally functionalized target peptide modified by an N-terminal functionalizing reagent is used, wherein:
a) the N-terminally functionalized target peptide has a formula: Z-P1-P2-peptide, wherein Z is an N-terminal modification capable of coordinating or chelating a zinc metal cation M, P1-P2-peptide is a target peptide before modification with the N-terminal modifier agent, Z-P1 is a functionalized N-terminal amino acid (NTAA) residue of the target peptide, and P2 is a penultimate terminal amino acid residue of the target peptide;
b) the engineered metalloprotein binding agent specifically binds to the N-terminally functionalized target peptide through interaction between the engineered metalloprotein binding agent and the Z-P1 (or Z-P1-P2) of the N-terminally functionalized target peptide; and
c) the engineered metalloprotein binding agent comprises an amino acid sequence X1-C/H/D/E-X2-C/H/D/E-X3-C/H/D/E-X4, wherein C/H/D/E is any single amino acid residue independently selected from the group consisting of amino acid residues C (Cys), H (His), D (Asp) and E (Glu); X1, X2, X3 and X4 are each any amino acid sequence comprising between 0 and 200 amino acid residues in length, and wherein the amino acid sequence X1-C/H/D/E-X2-C/H/D/E-X3-C/H/D/E-X4 chelates a zinc metal cation M with a thermodynamic dissociation constant (Kd) of 0.5 nM or less.

In some embodiments, binding agents can be developed through directed evolution of scaffolds with 10 μM or lower affinity using phage display, as disclosed in US patent publications US 2022/0283175 A1 and US 2023/0220589 A1, incorporated herein by reference.

In some preferred embodiments, the engineered metalloprotein binding agent comprises an amino acid sequence having at least about 80%, 90% or 95% sequence homology to any one of the amino acid sequences set forth in SEQ ID NO: 54-SEQ ID NO: 59. In some other embodiments, the engineered metalloprotein binding agents are used that are disclosed in in US patent publication US 2022/0283175 A1, incorporated herein by reference.

In some embodiments, a plurality of binding agents is a plurality of aptamers, wherein each aptamer from the plurality of aptamers exhibits binding specificity toward at least one N-terminal amino acid residue of a polypeptide immobilized on a solid support. Generation of such aptamers are disclosed in US 20210079557 A1, incorporated herein by reference. In other embodiments, each aptamer from the plurality of aptamers exhibits binding specificity towards particular epitopes of a macromolecular analyte immobilized on a solid support.

In certain embodiments, a binding agent may be designed to bind covalently. Covalent binding can be designed to be conditional or favored upon binding specifically to a moiety of the macromolecule analyte. For example, a target and its cognate binding agent may each be modified with a reactive group such that once the target-specific binding agent is bound to the target, a coupling reaction is carried out to create a covalent linkage between the two. Non-specific binding of the binding agent to other locations that lack the cognate reactive group would not result in covalent attachment. In some embodiments, the macromolecule analyte can form a covalent bond to a binding agent. Covalent binding between a binding agent and its target may allow for more stringent washing to be used to remove binding agents that are non-specifically bound, thus increasing the specificity of the assay. In some embodiment, the method further includes performing one or more wash steps. In some embodiments, the method includes a wash step after contacting the binding agent to the polypeptides to remove non-specifically bound binding agents. The stringency of the wash step may be tuned depending on the affinity of the binding agent to the polypeptides.

In some embodiments, a binding agent may preferably bind to a chemically modified or functionalized amino acid. In certain embodiments, a binding agent may be a selective binding agent. As used herein, selective binding refers to the ability of the binding agent to preferentially bind to a specific ligand (e.g., amino acid or group/class of amino acids) relative to binding to a different ligand (e.g., amino acid or group/class of amino acids). Selectivity is commonly referred to as the equilibrium constant for the reaction of displacement of one ligand by another ligand in a complex with a binding agent. Typically, such selectivity is associated with the spatial geometry of the ligand and/or the manner and degree by which the ligand binds to a binding agent, such as by hydrogen bonding, hydrophobic binding, and Van der Waals forces (non-covalent interactions) or by reversible or non-reversible covalent attachment to the binding agent. It should also be understood that selectivity may be relative, and as opposed to absolute, and that different factors can affect the same binding agent, including ligand concentration. In some embodiments, the binding agent is partially specific or selective. In some embodiments, the binding agent preferentially binds one or more functionalized amino acid residues. In some examples, a binding agent may bind to or is capable of binding to two or more of the twenty functionalized canonical amino acid residues. For example, one binding agent of the plurality of binding agents selectively binds only to a single functionalized amino acid residue from the twenty functionalized amino acid residues, whereas another binding agent of the plurality of binding agents selectively binds to three different functionalized amino acid residues from the twenty functionalized amino acid residues.

In some embodiments, a binding agent may have a preference for one or more specific functionalized terminal amino acids and have a flexible preference for a target at the penultimate position. In some other examples, a binding agent may have a preference for one or more specific target amino acids in the penultimate amino acid position and have a flexible preference for a target at the terminal amino acid position. In some embodiments, a binding agent is selective for a target comprising a terminal amino acid and other components of a macromolecule. In some particular embodiments, a binding agent is selective for a target comprising a terminal amino acid and an amide peptide backbone. In some cases, the peptide backbone comprises a natural peptide backbone or a post-translational modification. In some embodiments, the binding agent exhibits allosteric binding.

In some embodiments, binding between the binding agent and polypeptide or portion thereof is sufficient for the provided methods as long as it allows the first RNAP portion A and RNAP portion Bs to be brought into sufficient proximity to generate a functional RNAP enzyme. In the practice of the methods disclosed herein, the ability of a cognate binding agent to selectively bind a particular NTAA need only be sufficient to generate a signal that is distinguishable from signals generated by other, non-cognate binding agents, such that it's sufficient to generate a functional RNAP enzyme that produces a specific RNA transcript. In certain embodiments, a binding agent has a Kd of about or less than 500 nM, less than 200 nM, less than 100 nM, less than 50 nM, less than 10 nM, less than 5 nM, or less than 1 nM. In a particular embodiment, the binding agent is added to the polypeptide at a concentration >1×, >5×, >10×, >100×, or >1000×its Kd to drive binding to saturation. For example, the binding kinetics of an antibody to a single protein molecule is described in Chang et al., J Immunol Methods (2012) 378(1-2): 102-115. In a particular embodiment, the provided methods for performing a binding reaction are compatible with a binding agent with medium to low affinity for the macromolecule analyte. In preferred embodiments, the binding affinity is higher than the background affinity of RNAP portion A binding to RNAP portion B, in order for signal to be above noise.

In certain embodiments, a lectin is used as a binding agent for detecting the glycosylation state of a protein, polypeptide, or peptide. Lectins are carbohydrate-binding proteins that can selectively recognize glycan epitopes of free carbohydrates or glycoproteins. In some embodiments, the binding agent may be a modified aminopeptidase that has been engineered to recognize a functionalized terminal amino acid residue (such as modified from aminopeptidases disclosed in WO1988005993 A2, incorporated herein by reference).

A binding agent can be made by modifying naturally-occurring or synthetically-produced proteins by genetic engineering to introduce one or more mutations in the amino acid sequence to produce engineered proteins that bind to a specific component or feature of a polypeptide (e.g., NTAA, C-terminal amino acid (CTAA), or post-translationally modified amino acid or a peptide). For example, exopeptidases (e.g., aminopeptidases, carboxypeptidases), exoproteases, mutated exoproteases, mutated anticalins, mutated ClpSs, antibodies, or tRNA synthetases can be modified to create a binding agent that selectively binds to a particular NTAA. Generation of protein-based specific NTAA binding agents are disclosed in U.S. Pat. No. 9,435,810 B2, U.S. Ser. No. 11/634,709 B2, US 2023/0279386, U.S. Ser. No. 11/427,814 B2, U.S. Ser. No. 11/788,080 B2, WO 2020/223000, and provisional U.S. application 63/085,977, each of which is incorporated by reference in its entirety for all purposes. In another example, carboxypeptidases can be modified to create a binding agent that selectively binds to a particular CTAA. A binding agent can also be designed or modified, and utilized, to specifically bind a functionalized NTAA or modified CTAA, for example one that has a post-translational modification (e.g., phosphorylated NTAA or phosphorylated CTAA) or one that has been modified with a label (e.g., a chemical reagent). Strategies for directed evolution of proteins are known in the art (e.g., Yuan et al., 2005, Microbiol. Mol. Biol. Rev. 69:373-392), and include, but are not limited to, phage display, ribosomal display, mRNA display, CIS display, CAD display, emulsions, cell surface display method, yeast surface display, bacterial surface display, phage-assisted continuous evolution, etc.

In some embodiments, a binding agent may bind to a native or unmodified or unlabeled terminal amino acid. Moreover, in some cases, these natural amino acid binding agents do not recognize N-terminal labels or chemical functionalizations. Directed evolution of aminoacyl tRNA synthetase (aaRS) scaffolds can be used to generate higher affinity, higher specificity binding agents that recognized the N-terminal amino acids in the context of an N-terminal label. In another example, Havranek et al. (U.S. Pat. No. 10,852,305 B2, incorporated herein by reference) describes engineering aminoacyl tRNA synthetases (aaRSs) as specific NTAA binding agents. The amino acid binding pocket of the aaRSs has an intrinsic ability to bind cognate amino acids, but generally exhibits poor binding affinity and specificity. Moreover, these natural amino acid binding agents do not recognize N-terminal labels. Directed evolution of aaRS scaffolds can be used to generate higher affinity, higher specificity binding agents that recognized the N-terminal amino acids in the context of an N-terminal label.

In some embodiments of the disclosed methods, no N-terminal functionalization is used before the binding reaction or binding step, and the binding agents could be N-terminal binding proteins, C-terminal binding proteins, or peptide-binding proteins (e.g., aminoacyl tRNA synthetases with their tRNA-binding domains truncated, carboxypeptidase T, PDZ domains, or a number of peptide fragment-binding and peptide terminus-binding proteins). During the NTAA cleavage step, N-terminus workflow chemistries such as Edman degradation or PMI chemistry could be utilized, since the RNAP portion A can be renewed between cycles. Under certain chemical conditions (such as acidic conditions), the nucleic acid recording tag on the bead could comprise a locked nucleic acid (LNA) (or peptide nucleic acid (PNA), or other modified nucleic acids) instead of DNA-based nucleic acid for improved chemical stability, as long as it can still act as a template for transcription of RNA transcripts by the functional RNA polymerase enzyme.

In some embodiments, low affinity of truncated aminoacyl tRNA synthetases for various NTAA residues may be allowable in the disclosed methods, since the ratio of RNA or cDNA with a certain length in a given binding cycle to all RNA or cDNA in a given cycle determines the predicted NTAA probability during the analysis. Thus, as long as the affinity of the truncated aminoacyl tRNA synthetases for various NTAA residues is higher than the RNAPN-RNAPC (RNAP portion A-RNAP portion B) interaction affinity, the signal-to-noise ratio of encoding events is biased towards the unique NTAA residue presented by the polypeptide on the bead. In some embodiments, any NTAA-binding or C-terminal-binding protein fused to RNAPC (RNAP portion B) variants could be a suitable binding agent in the disclosed methods as long as the binding agent affinity for the polypeptide N-terminus or C-terminus is higher than the RNAPN-RNAPC (RNAP portion A-RNAP portion B) interaction affinity.

In some embodiments, a macromolecule analyte can be modified/functionalized before the step of contacting the macromolecule analyte with the binding agent. In some cases, the polypeptide can be modified/functionalized after generating RNA transcripts, prior to repeating the step of contacting the polypeptide with another cycle of a binding agent or a plurality of binding agents. In some embodiments, a binding agent may bind to a chemically modified or enzymatically modified NTAA residue. In some embodiments, the polypeptide or a portion thereof is labeled with a reagent selected from the group consisting of a phenyl isothiocyanate (PITC), a nitro-PITC, a sulfo-PITC, a phenyl isocyanate (PIC), a nitro-PIC, a sulfo-PIC, benzyloxycarbonyl chloride or carbobenzoxy chloride (Cbz-Cl), N-(Benzyloxycarbonyloxy)succinimide (Cbz-OSu or Cbz-O-NHS), a carboxyl-activated amino-blocked amino acid (e.g., Cbz-amino acid-OSu), a 1-fluoro-2,4-dinitrobenzene (Sanger's reagent, DNFB), dansyl chloride (DNS-Cl, or 1-dimethylaminonaphthalene-5-sulfonyl chloride), 4-sulfonyl-2-nitrofluorobenzene (SNFB), an anhydride, 2-Pyridinecarboxaldehyde, 2-Formylphenylboronic acid, 2-Acetylphenylboronic acid, 1-Fluoro-2,4-dinitrobenzene, 4-Chloro-7-nitrobenzofurazan, Pentafluorophenylisothiocyanate, 4-(Trifluoromethoxy)-phenylisothiocyanate, 4-(Trifluoromethyl)-phenylisothiocyanate, 3-(Carboxylic acid)-phenylisothiocyanate, 3-(Trifluoromethyl)-phenylisothiocyanate, 1-Naphthylisothiocyanate, N-nitroimidazole-1-carboximidamide, N,N′-Bis(pivaloyl)-1H-pyrazole-1-carboxamidine, N,N′-Bis(benzyloxycarbonyl)-1H-pyrazole-1-carboxamidine, an acetylating reagent, a guanidinylation reagent, a thioacylation reagent, a thioacetylation reagent, a thiobenzylation reagent, a diheterocyclic methanimine reagent, or a derivative thereof. In some embodiments, the polypeptide is labeled with an anhydride or derivative thereof. In some examples, the binding agent binds to an amino acid labeled by contacting it with a reagent (such as a macromolecule or a small molecule) or using a method as described in U.S. Patent Publications No. US 20200348307 A1 or US 2022/0227889 A1, incorporated herein. In some particular embodiments, the binding agent binds an amino acid labeled by an amine modifying reagent.

Other potential scaffolds that can be engineered to generate binding agents for use in the methods described herein include: an anticalin, a lipocalin, an amino acid tRNA synthetase (aaRS), ClpS, an Affilin®, an Adnectin™, a T cell receptor, a zinc finger protein, a thioredoxin, GST A1-1, DARPin, an affimer, an affitin, an alphabody, an avimer, a monobody, an antibody, a single domain antibody, a nanobody, see e.g., El-Gebali et al., (2019) Nucleic Acids Research 47:D427—D432 and Finn et al., (2013) Nucleic Acids Res. 42 (Database issue):D222—D230. In some embodiments, a binding agent is derived from an enzyme which binds one or more amino acids (e.g., an aminopeptidase).

In some embodiments, binding specificity between an engineered binding agent and an N-terminally modified target peptide is predominantly or substantially determined by the interaction between the engineered binding agent and the modified NTAA residue of the N-terminally modified target peptide, which means that there is only minimal or no interaction between the engineered binding agent and the penultimate terminal amino acid residue (P2) of the target peptide, as well as other residues of the target peptide. In some embodiments, the engineered binding agent binds with at least 5-fold higher binding affinity to the modified NTAA residue of the target peptide than to any other region of the target peptide. In some embodiments, the engineered binding agent has a substrate binding pocket with certain size and/or geometry matching the size and/or geometry of the modified NTAA residue of the N-terminally modified target peptide, to which the engineered binding agent specifically binds. In such embodiments, the modified NTAA residue occupies a volume encompassing a substrate binding pocket of the engineered binding agent that effectively precludes the P2 residue of the target peptide from entering into the substrate binding pocket or interacting with affinity-determining residues of the engineered binding agent. In some embodiments, the engineered binding agent specifically binds to N-terminally modified target peptides, wherein the target peptides share the same modified NTAA residue that interacts with the engineered binding agent, but have different P2 residues. In some embodiments, the engineered binding agent is capable of specifically binding to each N-terminally modified target peptide from a plurality of N-terminally modified target peptides, wherein the plurality of N-terminally modified target peptides contains at least 3, at least 5, or at least 10 N-terminally modified target peptides that were modified with the same N-terminal modifier agent, have the same modified NTAA residue, and have different P2 residues. Thus, in preferred embodiments, the engineered binding agent possesses binding affinity towards the modified NTAA residue of the N-terminally modified target peptide, but has little or no affinity towards P2 or other residues of the target peptide.

The functional affinity (avidity) of a given monovalent binding agent may be increased by at least an order of magnitude by using a bivalent or higher order multimer of the monovalent binding agent (Vauquelin et al., 2013, Br J Pharmacol 168(8): 1771-1785. 2013). In some embodiments, the binding agent is linked, directly or indirectly, to a multimerization domain. Thus, monomeric, dimeric, and higher order (e.g., 3, 4, 5, or more) multimeric polypeptides comprising one or more binding agents are provided herein. In some specific embodiments, the binding agent is dimeric. In some examples, two polypeptides can be covalently or non-covalently attached to each other to form a dimer.

In certain embodiments, the concentration of the binding agents joined to an RNAP portion B in a solution is controlled to reduce background and/or false positive results of the assay. In some embodiments, the concentration of a binding agent joined to an RNAP portion B can be at any suitable concentration, e.g., at about 0.0001 nM, about 0.001 nM, about 0.01 nM, about 0.1 nM, about 1 nM, about 2 nM, about 5 nM, about 10 nM, about 20 nM, about 50 nM, about 100 nM, about 200 nM, about 500 nM, about 1,000 nM, about 10,000 nM, or between any of these values.

In some embodiments, the ratio between the soluble binding agent molecules and the immobilized polypeptides can be at any suitable range, e.g., at about 0.00001:1, about 0.0001:1, about 0.001:1, about 0.01:1, about 0.1:1, about 1:1, about 2:1, about 5:1, about 10:1, about 15:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, about 10³:1, about 10⁴:1, about 10⁵:1, about 10⁶:1, or any ratio in between the above listed ratios. Higher ratios between the soluble binding agent molecules and the immobilized polypeptide(s) and/or the nucleic acids can be used to drive the binding. This may be particularly useful for detecting and/or analyzing low abundance polypeptides in a sample.

In some embodiments, the functionalization of the NTAA residue of the immobilized polypeptide analyte and the cleaving of the functionalized NTAA residue are performed according to methods disclosed in the published patent publication No. US 2022/0227889 A1. Scheme I shows an exemplary functionalization of the peptide NTAA residue to form compounds of Formula (II), followed by inducing elimination of the functionalized NTAA under mild conditions at around pH 5-10.

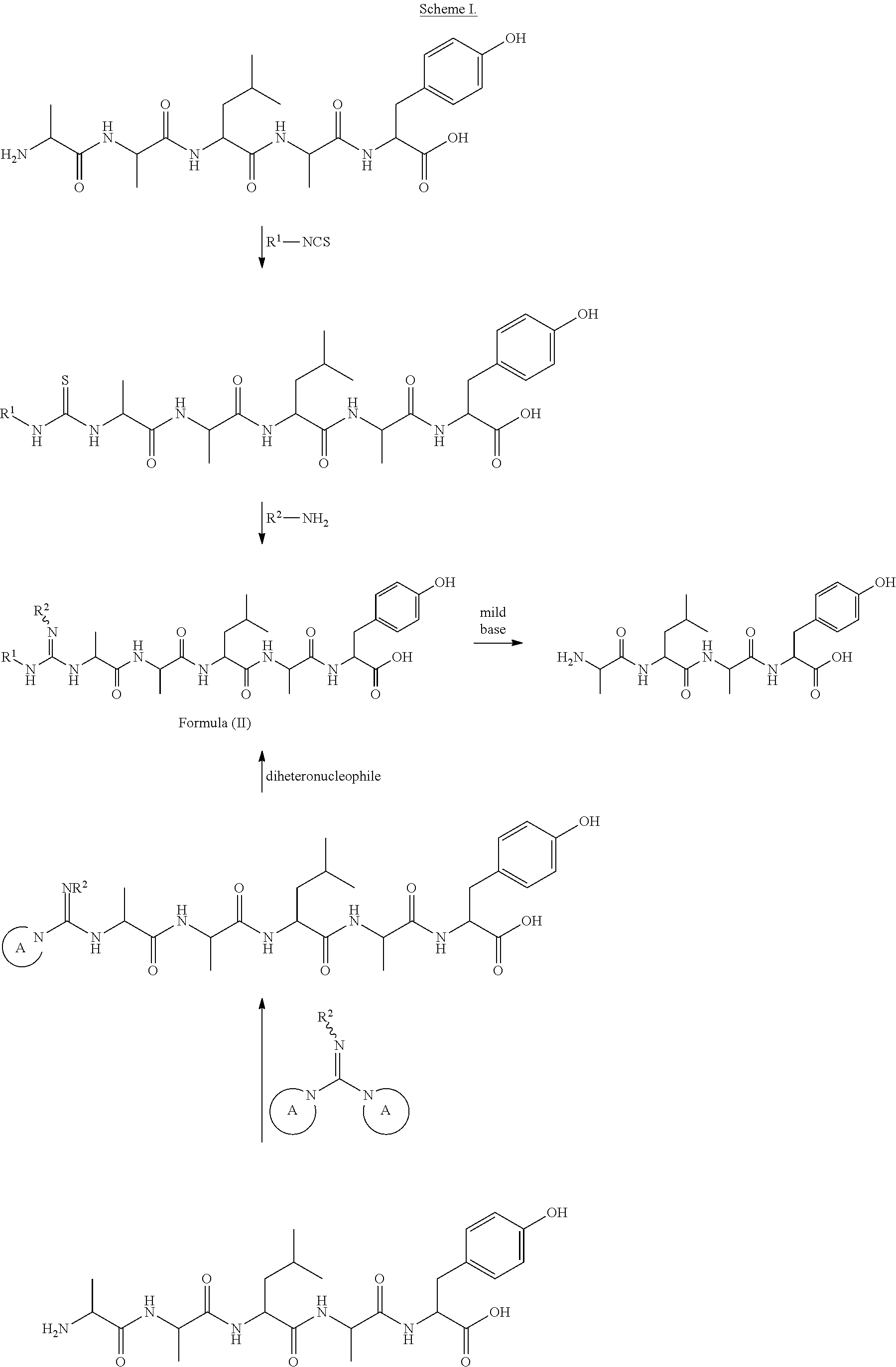

The reactions shown in Scheme I result in cleavage of the NTAA from a peptide under mild conditions, and thus enable a method for removal of the NTAA from a peptide. The described method can be used repeatedly, to remove one NTAA at a time from the immobilized peptide. The mild reaction conditions involved make it possible to perform these reactions in the presence of acid-sensitive moieties, such as nucleic acid recording tags. The nucleic acids are stable to the conditions used for functionalization and cleavage of the NTAA of a peptide as shown by data presented in the published patent application US 2022/0227889 A1, incorporated herein by reference.

In some embodiments, functionalization of the NTAA using a chemical reagent comprising a compound of Formula (AA) and the subsequent elimination are as depicted in the following scheme:

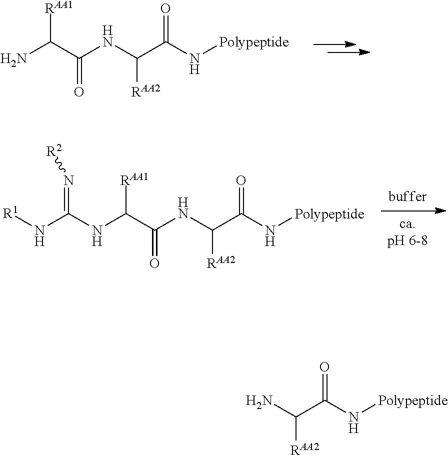

wherein R¹ and R² are as defined above and R^{AA1} is the side chain of the NTAA of a peptide.

In some embodiments, the functionalized NTAA is removed by a suitable reagent. The mixture is typically maintained at 25° C.-100° C. for 10-60 minutes in the medium to effect removal of the NTAA. An example of a suitable medium is water with phosphate, sodium chloride, Tween-20 (surfactant) at pH 5-10, and is heated at 25° C.-60° C. for 1 to 60 minutes containing a suitable reagent such as a diheteronucleophile. In some embodiments, the elimination is performed using an aqueous formulation that includes 0.1M to 2.0M sodium, potassium, cesium, or ammonium phosphate buffer or sodium, potassium, or ammonium carbonate buffer at a pH 5.5-9.5 at 50-100° C. for 5-60 minutes. In some embodiments, the suitable reagent for NTAA elimination comprises a hydroxide, ammonia, or a diheteronucleophile, typically at a concentration of 0.15 M-4.5 M.

In other embodiments, cleaving the modified NTAA residue of the peptide can be achieved by methods disclosed in US 2020/0348307 A1, incorporated herein by reference.

In yet other embodiments, cleaving the modified NTAA residue of the peptide is achieved by using an engineered enzyme, such as an engineered dipeptidyl aminopeptidase disclosed in the published patent application US 2021/0214701 A1, incorporated herein by reference.

In preferred embodiments of the disclosed methods and systems, cleaving the modified NTAA residue of the peptide is done by an engineered enzyme, such as a modified cleavase, which is configured to cleave a peptide bond between a terminally labeled amino acid residue and a penultimate terminal amino acid residue of a polypeptide, wherein the modified cleavase is derived from a dipeptidyl aminopeptidase, which removes an unlabeled terminal dipeptide from a polypeptide, wherein the dipeptide aminopeptidase comprises an amino acid sequence having at least 20% sequence identity to the amino acid sequence of SEQ ID NO: 60 and also comprising an asparagine residue at a position corresponding to position 191 of SEQ ID NO: 60, a tryptophan residue or phenylalanine residue at a position corresponding to position 192 of SEQ ID NO: 60, an arginine residue at a position corresponding to position 196 of SEQ ID NO: 60, an asparagine residue at a position corresponding to position 306 of SEQ ID NO: 60, and an aspartate residue at a position corresponding to position 650 of SEQ ID NO: 60; and wherein the modified cleavase comprises two or more amino acid substitutions in the residues corresponding to positions N191, W/F192, R196, N306, and D650 of SEQ ID NO: 60, as disclosed in the patent application US 2021/0214701 A1.

In some other preferred embodiments, a set of modified cleavases, comprising at least two different modified cleavases, is used to cleave the modified NTAA residue of the peptide, wherein:
(i) each of the modified cleavases from the set of modified cleavases is configured to cleave a peptide bond between a terminally labeled amino acid residue and a penultimate terminal amino acid residue of a polypeptide, wherein the modified cleavase is derived from a dipeptidyl aminopeptidase, which removes an unlabeled terminal dipeptide from a polypeptide, wherein the dipeptide aminopeptidase comprises an amino acid sequence having at least 20% sequence identity to the amino acid sequence of SEQ ID NO: 60 and also comprising an asparagine residue at a position corresponding to position 191 of SEQ ID NO: 60, a tryptophan residue or phenylalanine residue at a position corresponding to position 192 of SEQ ID NO: 60, an arginine residue at a position corresponding to position 196 of SEQ ID NO: 60, an asparagine residue at a position corresponding to position 306 of SEQ ID NO: 60, and an aspartate residue at a position corresponding to position 650 of SEQ ID NO: 60; and wherein the modified cleavase comprises two or more amino acid substitutions in the residues corresponding to positions N191, W/F192, R196, N306, and D650 of SEQ ID NO: 60; and
(ii) the modified cleavases from the set of modified cleavases have different specificities for terminally labeled amino acids, which the modified cleavases are configured to remove, as disclosed in the patent application US 2021/0214701 A1.

In preferred embodiments, the modified cleavase does not remove an unlabeled (unfunctionalized) terminal amino acid or dipeptide from the polypeptide.

In preferred embodiments, the modified cleavase comprises at least three amino acid substitutions in the residues corresponding to positions N191, W/F192, R196, N306, and D650 of SEQ ID NO: 60.

Multiple embodiments can be implemented for a specific arrangement and type of the nucleotide moiety used during the encoding process and representing the encoded structural information, such as modified nucleotides to produce a modified nucleic transcript output.

Monitoring of RNA transcripts generated by the disclosed methods can be performed on any number of commercially available devices. Moreover, existing devices may be modified or adapted for use in the methods of the present invention.

In some embodiments, the method further includes removing a portion of the polypeptide. In some embodiments, the method includes removing the terminal amino acid from the peptide, thereby yielding a newly exposed terminal amino acid, and contacting with a binding agent may be repeated on the newly exposed terminal amino acid. Removal of a portion of the polypeptide, e.g., a terminal amino acid such as a NTAA, may be accomplished by any number of known techniques, including chemical and enzymatic techniques. In some embodiments, the repeated steps for analyzing the newly exposed NTAA are substantially similar to the first cycle, including contacting with a binding agent capable of binding to the newly exposed NTAA and associated with an RNAP portion B, and detecting the signal generated by the detectable label formed when binding of the newly exposed NTAA by the binding agent brings the RNAP portion A and the RNAP portion B into sufficient proximity. In some cases, it may be beneficial to wash the polypeptide with, for example, a suitable buffer to remove and/or dissociate components between steps.

In embodiments relating to methods of analyzing target peptides or polypeptides using a degradation based approach, following contacting and binding of a first binding agent to an n NTAA of a peptide of n amino acids and detecting the signal generated, the n NTAA is eliminated. Removal of the n labeled NTAA by contacting with an enzyme or chemical reagents converts the n−1 amino acid of the peptide to an N-terminal amino acid, which is referred to herein as an n−1 NTAA. A second binding agent is contacted with the peptide and binds to the n−1 NTAA, and the signal generated is detected. In some embodiments, a signal or a lack of signal generated by the detectable label is observed and/or detected. Elimination of the n−1 labeled NTAA converts the n−2 amino acid of the peptide to an N-terminal amino acid, which is referred to herein as n−2 NTAA. Additional binding and detection can occur as described above up to n amino acids, wherein the observed signals over two or more cycles collectively represent the peptide. As used herein, an n “order” when used in reference to a binding agent refers to the n binding cycle. In some embodiments, one or more wash steps are performed before, within, or after each cycle. In some embodiments, steps including the NTAA in the described exemplary approach can be performed instead with a C terminal amino acid (CTAA).

Enzymatic cleavage of a NTAA may be accomplished by a peptidase, e.g., a carboxypeptidase, aminopeptidase, or dipeptidyl peptidase, dipeptidyl aminopeptidase, or variant, mutant, or modified protein thereof. Aminopeptidases naturally occur as monomeric and multimeric enzymes, and may be metal or ATP-dependent. Natural aminopeptidases have very limited specificity, and generically cleave N-terminal amino acids in a processive manner, cleaving one amino acid off after another. For the methods described here, aminopeptidases (e.g., metalloenzymatic aminopeptidase) may be engineered to possess specific binding or catalytic activity to the NTAA only when modified with an N-terminal label. For example, an aminopeptidase may be engineered such than it only cleaves an N-terminal amino acid if it is modified by a group such as PTC, modified-PTC, Cbz, DNP, SNP, acetyl, guanidinyl, diheterocyclic methanimine, etc. In this way, the aminopeptidase cleaves only a single amino acid at a time from the N-terminus, and allows control of the degradation cycle. In some embodiments, the modified aminopeptidase is non-selective as to amino acid residue identity while being selective for the N-terminal label. In other embodiments, the modified aminopeptidase is selective for both amino acid residue identity and the N-terminal label.

Engineered aminopeptidase mutants that bind to and cleave individual or small groups of labelled (biotinylated) NTAAs have been described (see, PCT Publication No. WO2010/065322, incorporated by reference in its entirety). Aminopeptidases are enzymes that cleave amino acids from the N-terminus of proteins or peptides. Natural aminopeptidases have very limited specificity, and generically eliminate N-terminal amino acids in a processive manner, cleaving one amino acid off after another (Kishor et al., 2015, Anal. Biochem. 488:6-8). However, residue specific aminopeptidases have been identified (Eriquez et al., J. Clin. Microbiol. 1980, 12:667-71; Wilce et al., 1998, Proc. Natl. Acad. Sci. USA 95:3472-3477; Liao et al., 2004, Prot. Sci. 13:1802-10). Aminopeptidases may be engineered to specifically bind to 20 different NTAAs representing the standard amino acids that are labeled with a specific moiety (e.g., PTC, DNP, SNP, etc.). Control of the stepwise degradation of the N-terminus of the peptide is achieved by using engineered aminopeptidases that are only active (e.g., binding activity or catalytic activity) in the presence of the label. In another example, Havranak et al. (U.S. Patent Publication No. US 2014/0273004) describes engineering aminoacyl tRNA synthetases (aaRSs) as specific NTAA binding agents. The amino acid binding pocket of the aaRSs has an intrinsic ability to bind cognate amino acids, but generally exhibits poor binding affinity and specificity. Moreover, these natural amino acid binding agents do not recognize N-terminal labels. Directed evolution of aaRS scaffolds can be used to generate higher affinity, higher specificity binding agents that recognized the N-terminal amino acids in the context of an N-terminal label.

For embodiments relating to CTAA binding agents, methods of cleaving CTAA from peptides are also known in the art. For example, U.S. Pat. No. 6,046,053 discloses a method of reacting the peptide or protein with an alkyl acid anhydride to convert the carboxy-terminus into oxazolone, liberating the C-terminal amino acid by reaction with acid and alcohol or with an ester. Enzymatic cleavage of a CTAA may also be accomplished by a carboxypeptidase. Several carboxypeptidases exhibit amino acid preferences, e.g., carboxypeptidase B preferentially cleaves at basic amino acids, such as arginine and lysine. As described above, carboxypeptidases may also be modified in the same fashion as aminopeptidases to engineer carboxypeptidases that specifically bind to CTAAs having a C-terminal label. In this way, the carboxypeptidase cleaves only a single amino acid at a time from the C-terminus, and allows control of the degradation cycle. In some embodiments, the modified carboxypeptidase is non-selective as to amino acid residue identity while being selective for the C-terminal label. In other embodiments, the modified carboxypeptidase is selective for both amino acid residue identity and the C-terminal label.

In some embodiments, the polypeptide is contacted with one or more additional enzymes to eliminate the NTAA (e.g., a proline aminopeptidase to remove an N-terminal proline, if present). In some embodiments, the enzyme eliminates an NTAA from the polypeptide that is a proline. In some specific examples, the enzyme is a proline aminopeptidase, a proline iminopeptidase (PIP), or a pyroglutamate aminopeptidase (pGAP). In some embodiments, the enzymes to treat the polypeptides can be used in combination with a chemical or enzymatic methods for removing/eliminating amino acids from the polypeptide. In some cases, enzymes can be provided in combinations or mixtures. PAP enzymes that cleave N-terminal prolines are also referred to as proline iminopeptidases (PIPs). Known monomeric PAPs include family members from B. coagulans, L. delbrueckii, N. gonorrhoeae, F. meningosepticum, S. marcescens, T. acidophilum, L. plantarum (MEROPS 533.001) Nakajima et al., J Bacteriol. (2006) 188(4):1599-606; Kitazono et al., Bacteriol (1992) 174(24):7919-7925). Known multimeric PAPs including D. hansenii (Bolumar et al., (2003) 86(1-2):141-151) and similar homologues from other species (Basten et al., Mol Genet Genomics (2005) 272(6):673-679). Either native or engineered variants/mutants of PAPs may be employed.

In some instances, the information from the provided methods can be stored, analyzed, and/or determined using a software tool. The software may utilize information about the binding characteristics of each binding agent. The software could also utilize a listing of some or all spatial locations in which each a signal was generated or not generated by the detectable label (a specific RNA transcript). In some embodiments, the software may comprise a database. The database may contain sequences of known proteins in the species from which the sample was obtained or also include related species (e.g., homologs). In some cases, if the species of the sample is unknown then a database of some or all protein sequences may be used. The database may also contain the characteristics and/or sequences of any known protein variants and mutant proteins thereof.

In some embodiments, the software may comprise one or more algorithms, such as a machine learning, deep learning, statistical learning, supervised learning, unsupervised learning, clustering, expectation maximization, maximum likelihood estimation, Bayesian inference, linear regression, logistic regression, binary classification, multinomial classification, or other pattern recognition algorithm. For example, the software may perform the one or more algorithms to analyze the information regarding (i) the binding characteristic of each binding agent used, (ii) information from the database of proteins, and/or (iii) a list of locations observed (including in different cycles), in order to generate or assign a probable identity to each signal detected (e.g., lengths and/or sequences of RNA transcripts) and/or a confidence (e.g., confidence level and/or confidence interval) for that information.

### EXEMPLARY EMBODIMENTS

Among the provided embodiments are:

1. A method for analyzing a macromolecule analyte, comprising:
a) contacting the macromolecule analyte with a plurality of binding agents,
wherein the macromolecule analyte is attached to a solid support and associated with (i) an RNA polymerase (RNAP) portion A, and (ii) a recording tag comprising a plurality of promoters,
wherein each binding agent comprises (i) an RNAP portion B, and (ii) a binding moiety, and wherein upon binding between a first motif of the macromolecule analyte and the binding moiety of a first binding agent of the plurality of binding agents, the RNAP portion A associated with the macromolecule analyte and the RNAP portion B of the first binding agent are brought into proximity to form a first functional RNAP, which initiates transcription from a corresponding first promoter of the plurality of promoters to generate a first RNA transcript;
b) collecting the first RNA transcript, and removing the first functional RNAP enzyme or a portion thereof from the recording tag associated with the macromolecule analyte;
c) contacting the macromolecule analyte with the plurality of binding agents,
wherein upon binding between a second motif of the macromolecule analyte and the binding moiety of a second binding agent of the plurality of binding agents, the RNAP portion A associated with the macromolecule analyte and the RNAP portion B of the second binding agent are brought into proximity to form a second functional RNAP, which initiates transcription from a corresponding second promoter of the plurality of promoters to generate a second RNA transcript; and
d) analyzing the first and second RNA transcripts, thereby analyzing the first and second motifs of the macromolecule analyte.
   2. The method of embodiment 1, wherein the plurality of promoters comprises at least four different promoters, and wherein the recording tag further comprises an analyte-specific barcode.
   3. The method of embodiment 2, wherein at least four binding agents from the plurality of binding agents have different RNAP portion Bs, and at least four different functional RNAP enzymes are formed via interactions between the RNAP portion A and each of the four different RNAP portion Bs joined to the at least four binding agents, wherein each of the at least four different functional RNAP enzymes is specific to a different promoter of the plurality of promoters.
   4. The method of any one of embodiments 1-3, wherein 100, 500, 1000 or more different macromolecule analytes are analyzed simultaneously, and each recording tag associated with one of the 100, 500, 1000 or more different macromolecule analytes comprises a different analyte-specific barcode.
   5. The method of any one of embodiments 1-4, wherein at step (d), the first RNA transcript and the second RNA transcript are analyzed by RNA or cDNA sequencing.
   6. The method of any one of embodiments 1-5, wherein the RNAP portion A associated with the macromolecule analyte is releasably or reversibly attached to the solid support, and/or
wherein the RNAP portion A associated with the macromolecule analyte is releasably or reversibly attached to the recording tag associated with the macromolecule analyte.
   7. The method of any one of embodiments 1-6, wherein the recording tag associated with the macromolecule analyte further comprises at least one transcription termination element.
   8. The method of any one of embodiments 1-7, wherein the recording tag associated with the macromolecule analyte further comprises at least one polyadenylation sequence.
   9. The method of any one of embodiments 1-8, wherein the binding moiety of each binding agent of the plurality of binding agents comprises a polypeptide or an aptamer.
   10. The method of any one of embodiments 1-9, further comprising attaching the macromolecule analyte to the recording tag and attaching the macromolecule analyte and/or the recording tag to the solid support before performing step (a).
   11. The method of any one of embodiments 1-10, wherein at step (a), the binding between the macromolecule analyte and the first binding agent is a non-covalent association.
   12. The method of any one of embodiments 1-11, wherein the recording tag associated with the macromolecule analyte consists of a single DNA molecule which comprises the plurality of promoters,
optionally wherein the single DNA molecule comprises one or more spacers between adjacent promoters, and
optionally wherein the recording tag associated with the macromolecule analyte comprises a plurality of hairpin structures, and each hairpin structure comprises a stem that comprises one or more of the plurality of promoters.
   13. The method of any one of embodiments 1-11, wherein the recording tag associated with the macromolecule analyte comprises a plurality of DNA molecules attached to the solid support, and wherein each DNA molecule of the plurality of DNA molecules comprises a different promoter of the plurality of promoters.
   14. The method of any one of embodiments 1-13, which is a cell-free method.
   15. The method of any one of embodiments 1-14, wherein the RNAP portion A is not covalently attached to the macromolecule analyte, optionally wherein the RNAP portion A is associated with the macromolecule analyte via a non-covalent binding pair, optionally wherein the non-covalent binding pair comprises a biotin or derivative or analog thereof and a streptavidin/avidin or derivative or analog thereof.
   16. The method of any one of embodiments 1-15, wherein the RNAP portion A comprises an amino acid sequence having at least 30% sequence identity to any one of the amino acid sequences set forth in SEQ ID NO: 5 to SEQ ID NO: 9, and the RNAP portion B comprises an amino acid sequence having at least 30% sequence identity to any one of the amino acid sequences set forth in SEQ ID NO: 10 to SEQ ID NO: 43.
   17. The method of any one of embodiments 1-16, wherein the RNAP portion A and each of the RNAP portion Bs of the plurality of binding agents are enzymatically inactive when not associated with each other, optionally wherein the RNAP portion A and each RNAP portion B, when not associated with each other, do not initiate transcription of a sequence in the recording tag.
   18. The method of any one of embodiments 1-17, wherein the RNAP portion A and each of the RNAP portion Bs of the plurality of binding agents have no RNA polymerase activity by itself.
   19. The method of any one of embodiments 1-18, wherein the RNAP portion A does not bind to a promoter of the plurality of promoters; and wherein each of the RNAP portion Bs of the plurality of binding agents is specific for a promoter of the plurality of promoters, but does not initiate transcription without binding to the RNAP portion A.
   20. The method of any one of embodiments 1-19, wherein the interaction between the RNAP portion A and the RNAP portion B to form the first or second functional RNAP enzyme is increased relative to the interaction between the RNAP portion A and the RNAP portion B without joining to the first or second binding agent, optionally wherein binding between the binding agent and the macromolecule analyte promotes and/or stabilizes the association between the RNAP portion A and the RNAP portion B.
   21. The method of any one of embodiments 1-20, wherein the first functional RNAP together with the first promoter and the second functional RNAP together with the second promoter form orthogonal pairs.
   22. The method of any one of embodiments 1-21, wherein the macromolecule analyte comprises a polypeptide analyte.
   23. The method of embodiment 22, wherein at step (d), at least a portion of an amino acid sequence of the polypeptide analyte is identified.
   24. The method of embodiment 22 or embodiment 23, wherein at least the first binding agent and the second binding agent are capable of binding to an N-terminal amino acid (NTAA) of the polypeptide analyte, or are capable of binding to an N-terminal amino acid (NTAA) of the polypeptide analyte functionalized with a chemical moiety.
   25. The method of embodiment 24, further comprising the following step: (0) modifying the NTAA of the polypeptide analyte with the chemical moiety to produce a functionalized NTAA of the polypeptide analyte, wherein the first binding agent is capable of binding to the modified NTAA of the polypeptide analyte, and step (0) is performed before step (a).
   26. The method of embodiment 24 or embodiment 25, further comprising the following step: (bc) removing a portion of the polypeptide analyte, wherein the removed portion of the polypeptide analyte comprises the NTAA, or the NTAA functionalized with the chemical moiety, thereby yielding a newly exposed NTAA of the polypeptide analyte, and wherein step (bc) is performed after step (b) and before step (c).
   27. The method of embodiment 26, wherein the portion of the polypeptide analyte removed at step (bc) comprises the NTAA functionalized with the chemical moiety, and the NTAA functionalized with the chemical moiety is removed from the polypeptide analyte by a modified cleavase enzyme.
   28. The method of embodiment 27, wherein the modified cleavase enzyme: (i) is configured to cleave a peptide bond between a terminally labeled amino acid residue and a penultimate terminal amino acid residue of a polypeptide; (ii) is derived from a dipeptidyl aminopeptidase, which removes an unlabeled terminal dipeptide from a polypeptide; (iii) comprises two or more amino acid substitutions in the residues corresponding to positions N191, W/F192, R196, N306, and D650 of SEQ ID NO: 60, wherein the dipeptide aminopeptidase comprises an amino acid sequence having at least 20% sequence identity to the amino acid sequence of SEQ ID NO: 60 and also comprising an asparagine residue at a position corresponding to position 191 of SEQ ID NO: 60, a tryptophan residue or phenylalanine residue at a position corresponding to position 192 of SEQ ID NO: 60, an arginine residue at a position corresponding to position 196 of SEQ ID NO: 60, an asparagine residue at a position corresponding to position 306 of SEQ ID NO: 60, and an aspartate residue at a position corresponding to position 650 of SEQ ID NO: 60.
   29. A system for analyzing a plurality of macromolecule analytes, comprising:
a) the plurality of macromolecule analytes attached to a solid support, wherein each macromolecule analyte from the plurality of macromolecule analytes is associated with (i) an RNA polymerase (RNAP) portion A, and (ii) a recording tag comprising a plurality of promoters comprising at least a first promoter and a second promoter; and
b) a plurality of binding agents capable of binding to a macromolecule analyte of the plurality of macromolecule analytes and comprising at least a first binding agent and a second binding agent, wherein each binding agent of the plurality of binding agents comprises (i) an RNAP portion B, and (ii) a binding moiety; wherein upon binding between a first motif of the macromolecule analyte and the binding moiety of the first binding agent, the RNAP portion A associated with the macromolecule analyte and the RNAP portion B of the first binding agent are brought into proximity to form a first functional RNAP, which initiates transcription from the first promoter to generate a first RNA transcript; and wherein
upon binding between a second motif of the macromolecule analyte and the binding moiety of the second binding agent, the RNAP portion A associated with the macromolecule analyte and the RNAP portion B of the second binding agent are brought into proximity to form a second functional RNAP, which initiates transcription from the second promoter to generate a second RNA transcript.
   30. The system of embodiment 29, wherein the RNAP portion B of the first binding agent is different from any other RNAP portion Bs of binding agents of the plurality of binding agents.
   31. The system of embodiment 29 or embodiment 30, wherein the binding moiety of the first binding agent is different from any other binding moieties of binding agents of the plurality of binding agents.
   32. The system of any one of embodiments 29-31, wherein the plurality of promoters comprises at least four different promoters, and wherein the recording tag comprises an analyte-specific barcode.
   33. The system of any one of embodiments 29-32, wherein the plurality of macromolecule analytes comprises at least 100, 1000, 10000 or more different macromolecule analytes, and each recording tag associated with one of the at least 100, 1000, 10000 or more different macromolecule analytes comprises a different analyte-specific barcode.
   34. The system of any one of embodiments 29-33, wherein the RNAP portion A comprises an amino acid sequence having at least 30% sequence identity to any one of the amino acid sequences set forth in SEQ ID NO: 5 to SEQ ID NO: 9, and the RNAP portion B comprises an amino acid sequence having at least 30% sequence identity to any one of the amino acid sequences set forth in SEQ ID NO: 10 to SEQ ID NO: 43.
   35. The system of any one of embodiments 29-34, wherein i) the plurality of binding agents comprises at least four different binding agents each comprising a different RNAP portion B; ii) each of the different RNAP portion Bs of the at least four different binding agents is specific to a different promoter of the plurality of promoters when each of the RNAP portion Bs is associated with the RNAP portion A to form a functional RNAP enzyme.
   36. The system of any one of embodiments 29-35, wherein the RNAP portion A associated with each macromolecule analyte from the plurality of macromolecule analytes is releasably or reversibly attached to the solid support, and/or wherein the RNAP portion A associated with the macromolecule analyte is releasably or reversibly attached to the recording tag associated with the macromolecule analyte.
   37. The system of any one of embodiments 29-36, wherein the recording tag associated with each macromolecule analyte from the plurality of macromolecule analytes further comprises at least one transcription termination element.
   38. The system of any one of embodiments 29-37, wherein the recording tag associated with each macromolecule analyte from the plurality of macromolecule analytes further comprises at least one polyadenylation sequence.
   39. The system of any one of embodiments 29-38, wherein the binding moiety of each binding agent of the plurality of binding agents comprises a polypeptide or an aptamer.
   40. The system of any one of embodiments 29-39, wherein the recording tag associated with each macromolecule analyte of the plurality of macromolecule analytes consists of a single DNA molecule comprising the plurality of promoters, optionally wherein the single recording tag molecule comprises one or more spacers between adjacent promoters.
   41. The system of any one of embodiments 29-39, wherein the recording tag associated with each macromolecule analyte of the plurality of macromolecule analytes comprises a plurality of DNA molecules attached to the solid support, and wherein each DNA molecule of the plurality of DNA molecules comprises a different promoter from the plurality of promoters.
   42. The system of any one of embodiments 29-41, wherein the RNAP portion A is not covalently attached to an associated macromolecule analyte, optionally wherein the RNAP portion A is associated with the macromolecule analyte via a non-covalent binding pair, optionally wherein the non-covalent binding pair comprises a biotin or derivative or analog thereof and a streptavidin/avidin or derivative or analog thereof.
   43. The system of any one of embodiments 29-42, wherein each of the RNAP portion As and each of the RNAP portion Bs of the plurality of binding agents are enzymatically inactive (e.g., do not initiate transcription of RNA) when not associated with each other, optionally wherein the RNAP portion A and each RNAP portion B, when not associated with each other, do not initiate transcription of a sequence in the recording tag.
   44. The system of any one of embodiments 29-43, wherein each of the RNAP portion As and each of the RNAP portion Bs have no RNA polymerase activity by itself.
   45. The system of any one of embodiments 29-44, wherein each of the RNAP portion As does not bind to a promoter of the plurality of promoters; and wherein each of the RNAP portion Bs of the plurality of binding agents is specific for a promoter of the plurality of promoters, but does not initiate transcription of RNA without binding to an RNAP portion A.
   46. The system of any one of embodiments 29-45, wherein the first functional RNAP together with the first promoter and the second functional RNAP together with the second promoter form orthogonal pairs.
   47. The system of any one of embodiments 29-46, wherein the plurality of macromolecule analytes comprises a plurality of polypeptide analytes.
   48. The system of embodiment 47, wherein each binding agent from the plurality of binding agents is capable of binding to an N-terminal amino acid (NTAA) of a polypeptide analyte from the plurality of polypeptide analytes, or is capable of binding to the NTAA functionalized with a chemical moiety.
   49. The system of embodiment 47 or embodiment 48, further comprising a functionalizing reagent capable of modifying an N-terminal amino acid (NTAA) residue of a polypeptide analyte to generate a functionalized NTAA residue of the polypeptide analyte, wherein one or more binding agents of the plurality of binding agents are capable of binding to the functionalized NTAA residue of the polypeptide analyte.
   50. The system of embodiment 48 or embodiment 49, further comprising an eliminating reagent for removing the NTAA residue or the functionalized NTAA residue of the polypeptide analyte.
   51. The system of any one of embodiments 47-50, further comprising an instruction for using the system in high-throughput polypeptide analysis or polypeptide sequencing.
   52. A method for analyzing a macromolecule analyte, comprising:
a) contacting the macromolecule analyte with a first binding agent,
wherein the macromolecule analyte is attached to a solid support and associated with (i) an RNA polymerase (RNAP) portion A, and (ii) a recording tag comprising a plurality of promoters,
wherein the first binding agent comprises (i) an RNAP portion B, and (ii) a binding moiety, and wherein upon binding between a first motif of the macromolecule analyte and the binding moiety of the first binding agent, the RNAP portion A associated with the macromolecule analyte and the RNAP portion B of the first binding agent are brought into proximity to form a first functional RNAP, which initiates transcription from a corresponding first promoter of the plurality of promoters to generate a first RNA transcript;
b) collecting the first RNA transcript;
c) removing the first binding agent from the macromolecule analyte, thereby removing the RNAP portion B from the recording tag and disrupting the first functional RNAP;
d) contacting the macromolecule analyte with a second binding agent,
wherein the second binding agent comprises (i) an RNAP portion B, and (ii) a binding moiety, and wherein upon binding between a second motif of the macromolecule analyte and the binding moiety of the second binding agent, the RNAP portion A associated with the macromolecule analyte and the RNAP portion B of the second binding agent are brought into proximity to form a second functional RNAP, which initiates transcription from a corresponding second promoter of the plurality of promoters to generate a second RNA transcript;
e) collecting the second RNA transcript; and
f) analyzing the first and second RNA transcripts, thereby analyzing the first and second motifs of the macromolecule analyte.
   53. The method of embodiment 52, wherein in step (a) the macromolecule analyte is contacted with a first plurality of binding agents comprising the first binding agent, and in step (d) the macromolecule analyte is contacted with a second plurality of binding agents comprising the second binding agent.
   54. The method of embodiment 53, wherein the first plurality of binding agents and the second plurality of binding agents are identical.
   55. The method of embodiment 53, wherein there is at least one different binding agent between the first plurality of binding agents and the second plurality of binding agents, and the binding agents differ in the identity of the binding moiety and/or the RNAP portion B.
   56. The method of any one of embodiments 52-55, wherein the first binding agent and the second binding agent are identical or different, optionally wherein the binding agents differ in the identity of the binding moiety and/or the RNAP portion B.
   57. The method of any one of embodiments 52-56, wherein the first motif and the second motif are identical.
   58. The method of any one of embodiments 52-56, wherein the first motif and the second motif are different.
   59. The method of any one of embodiments 52-57, wherein the second motif is available for binding by the second binding agent without cleaving or modifying the first motif.
   60. The method of any one of embodiments 52-57, wherein the second motif is generated or rendered available for binding by the second binding agent by cleaving or modifying the first motif.
   61. The method of any one of embodiments 52-60, wherein the first motif and/or the second motif comprise one or more terminal amino acid residues.
   62. The method of any one of embodiments 52-61, wherein the first functional RNAP and the second functional RNAP comprise identical RNAP portion A, and identical or different RNAP portion B.
   63. The method of any one of embodiments 52-62, wherein each of the first promoter and the second promoter is associated with a promoter-specific barcode.
   64. The method of any one of embodiments 52-63, wherein the first RNA transcript and the second RNA transcript are the same or different in length and/or sequence.

65. The method of any one of embodiments 52-64, wherein the first RNA transcript and the second RNA transcript comprise the same analyte-specific barcode and/or the same cycle-specific barcode.

### EXAMPLES

The following examples are offered to illustrate but not to limit the methods, compositions, and uses provided herein. Certain aspects of the present invention including, but not limited to, embodiments for the Proteocode™ peptide sequencing assay, methods of making nucleic acid-polypeptide conjugates, methods for attachment of nucleic acid-polypeptide conjugates to a solid support, methods of generating barcodes, methods of generating specific binding agents recognizing functionalized NTAA residues of a peptide, reagents and methods for modifying and/or removing NTAA residues from a peptide, methods for analyzing generated RNA transcripts were disclosed in the following published patent applications: US 2019/0145982 A1, US 2020/0348308 A1, US 2020/0348307 A1, US 2021/0208150 A1, US 2022/0049246 A1, U.S. Ser. No. 11/427,814, US 2022/0227889 A1, US 2022/0283175 A1, the contents of which are incorporated herein by reference in its entirety.

### Example 1. Peptide Analyte Immobilization Using Nucleic Acid Hybridization and Joining to a Solid Support

This example describes exemplary methods for joining (immobilizing) nucleic acid-peptide conjugates, such as conjugates of a peptide analyte with recording tag, to a solid support. These methods are the same as described in US 2022/0049246 A1. Brief description of the methods is provided below.

In a hybridization-based method of immobilization, nucleic acid-peptide conjugates were hybridized and ligated to hairpin capture DNAs that were chemically immobilized on magnetic beads. The capture nucleic acids were conjugated to the beads using trans-cyclooctene (TCO) and methyltetrazine (mTet)-based click chemistry. TCO-modified short hairpin capture nucleic acids (16 base pair stem, 5 base loop, 24 base 5′ overhang) were reacted with mTet-coated magnetic beads. Phosphorylated nucleic acid-peptide conjugates (10 nM) were annealed to the hairpin DNAs attached to beads in 5×SSC, 0.02% SDS, and incubated for 30 minutes at 37° C. The beads were washed once with PBST and resuspended in lx Quick ligation solution (New England Biolabs, USA) with T4 DNA ligase. After a 30-minute incubation at 25° C., the beads were washed twice with PBST and resuspended in the 50 μL of PBST.

Peptide analyte-recording tag conjugates immobilized on a solid support can be further associated with an RNAP portion A by introducing a modified nucleotide in the recording tag associated with the peptide analyte. A variety of CLICK-functionalized nucleotides for DNA labeling is known and commercially available, including C8-alkyne-dCTP, 5-DBCO-PEG4-dUTP, 5-azidomethyl-dUTP, C8-alkyne-dUTP, 5-TCO-PEG4-dUTP, and others. Other modified nucleotides comprising a handle for a click chemistry reaction are described in Fantoni N Z, et al., A Hitchhiker's Guide to Click-Chemistry with Nucleic Acids. Chem Rev. 2021 Jun. 23; 121(12):7122-7154, and Perrone D, et al., Modified Nucleosides, Nucleotides and Nucleic Acids via Click Azide-Alkyne Cycloaddition for Pharmacological Applications. Molecules. 2021 May 22; 26(11):3100, incorporated herein by reference. After immobilization of a peptide analyte on a recording tag attached to a solid support and having a modified nucleotide with a first reactive handle (e.g., azide, mTet, etc.), an RNAP portion A that comprises a second reactive handle complementary to the first reactive handle is introduced to the solid support (such as beads) and allowed to be attached to the recording tag. Any bioorthogonal reaction between the first reactive handle and the second reactive handle can be utilized, as long as the reaction conditions do not compromise integrity of the system components, such as they do not compromise integrity of the peptide analyte and nucleic acid recording tag. Accordingly, the peptide analyte attached to the solid support and associated with (i) the RNAP portion A, and (ii) the recording tag, is generated.

Alternatively, the RNAP portion A can be reversible attached to the recording tag associated with the peptide analyte. For example, both the RNAP portion A and the recording tag can be attached to affinity binding pairs (e.g., biotin-streptavidin binding pair, or a receptor-ligand binding pair), which creates a reversible attachment. The advantage of this approach is that the RNAP portion A can be replaced after one or several binding cycles (e.g., due to loss of integrity; the chemically modified or damaged RNAP portion A is washed away, while a new RNAP portion A is attached to the recording tag).

### Example 2. Generating a Set of Functional RNAP Enzymes Comprising RNAP Portion Bs with Orthogonal Promoter Specificity—Approach I

As described in Temme K, et al., 2012, homologues of T7 RNAP (SEQ ID NO:1) were identified and their DNA-binding preferences were computationally determined. 43 T7 RNAP homologues were identified from NCBI via a protein-protein BLAST against nonredundant protein sequences. A multiple sequence alignment of the RNAP amino acid sequences was performed using ClustalW to identify and extract the b-hairpin in each RNAP corresponding to T7 amino acids G732-P780. A second multiple sequence alignment was performed with only the b-hairpin sequences, and 13 RNAP subfamilies were identified (distance between members <0.1 in the ClustalW guide tree). Putative promoters were identified from each phage genome using PHIRE, software that scans genomes for regulatory elements by identifying conserved sequences with a limited number of user-defined degeneracies. WebLogo was used to determine the consensus sequence for each phage RNAP. For each b-hairpin subfamily, the binding region of the consensus promoter was identical (Temme K, et al., 2012). Novel RNAPs were generated by swapping the b-hairpin from T7 RNAP (Q744 to 1761) with the equivalent region from each subfamily. The corresponding binding region of the T7 promoter (−12C to −7C) was replaced with the promoter subfamily consensus sequences. The resulting RNAPs were screened for activity against their predicted promoters. Four RNAPs exhibited strong activity (42-, 12-, 17- and 40-fold induction by T7*, T7*(T3), T7*(K1F) and T7*(N4), respectively, which correspond to RNAP portion B sequences set forth in SEQ ID NOs: 10, 19, 41, and 42, respectively) (Temme K, et al., 2012). The activity of these RNAPs against non-cognate promoters was then characterized. Each RNAP is highly orthogonal, even after significant mutations to both the specificity loop and promoter. The data showed in Temme K, et al., 2012 indicates that the following RNAP fragments can be used in the disclosed methods: RNAP portion A—SEQ ID NO: 5; set of RNAP portion Bs—SEQ ID NOs: 10, 19, 41, and 42, wherein each of RNAP portion Bs is attached to a unique binding agent capable of binding to a portion of the macromolecule analyte. Binding between the RNAP portion A and each of the RNAP portion Bs produces four different functional RNAP enzymes configured to initiate transcription from the corresponding specific promoters comprising the following sequences: SEQ ID NO: 44, SEQ ID NO: 48, SEQ ID NO: 46, and SEQ ID NO: 51, which together comprise the plurality of promoters present on the recording tag.

### Example 3. Generating a Set of Functional RNAP Enzymes Comprising RNAP Portion Bs with Orthogonal Promoter Specificity—Approach II

A series of orthogonal RNAP portion Bs (RNAPC variants/mutants) can be generated as described in Pu J, et al., J Am Chem Soc. 2017. In the described split RNAP system, orthogonality comes from the RNAPC fragments that each drive transcription from unique DNA promoters located in the plurality of promoters upon RNAP enzyme assembly, thereby permitting the transcription of unique output RNA signals. Mutations within RNAPC fragments of enzymes that are homologous to T7 RNAP and that alter its DNA promoter specificity were analyzed (Segall-Shapiro T H, et al., A ‘resource allocator’ for transcription based on a highly fragmented T7 RNA polymerase. Mol Syst Biol. 2014 Jul. 30; 10(7):742). These variants were cloned into the E. coli luciferase reporter system (a reporter vector that produces luciferase in response to transcription from the T7 promoter) to assay their ability to function as proximity-dependent split RNAP reporters. The orthogonality was tested by measuring a series of eight putative RNAPC variants on a panel of five different DNA promoters, T7, CGG, K1F, CTGA, and T3. All of the variants displayed robust activity on their target promoter, but the variants differed in terms of overall activity and off-target activity on the other promoters (Pu J, et al., J Am Chem Soc. 2017). Based on overall activity and selectivity, a series of four, five, six or seven orthogonal RNAPC variants can be selected, along with their respective promoter sequences, for utilizing in the high-throughput polypeptide analysis methods disclosed herein.

In addition to Escherichia phage T7 (Bacteriophage T7) RNA polymerase (RNAP), herein referred to as (T7)RNAP, three additional RNAP homologs were selected that may be bisected (i.e. split) and used for the high-throughput polypeptide analysis technology: Enterobacteria phage T3 RNA polymerase, herein referred to as (T3)RNAP; Enterobacteria phage K11 RNA polymerase, herein referred to as (K11)RNAP; and Enterobacteria phage SP6 RNA polymerase, herein referred to as (SP6)RNAP (see SEQ ID NOs: 1-4). These four RNAP enzymes are capable of synthesizing RNA molecules from a DNA template molecule, and have been previously used in the art of biotechnology and molecular engineering. For selecting the split point (bisection point) in RNAP homologs, ClustalOmega software was used to produce a multiple sequence alignment of (T7)RNAP against (T3)RNAP, (K11)RNAP, and (SP6)RNAP. For each RNAP homolog, the split point was selected at the position corresponding to the (T7)RNAP split point position that was experimentally validated in Pu J, et al., J Am Chem Soc. 2017, namely: the RNAP portion A (N-terminal fragment), herein referred to as (T7)RNAPN, comprises residues 1-179 of (T7)RNAP, and the RNAP portion B (C-terminal fragment), herein referred to as (T7)RNAPC, comprises residues 180-883 of (T7)RNAP (SEQ ID NO: 5 and SEQ ID NO: 10, respectively). For (T3)RNAP, the RNAP portion A, herein referred to as (T3)RNAPN, comprises residues 1-180 of (T3)RNAP, and the RNAP portion B, herein referred to as (T3)RNAPC, comprises residues 181-884 of (T3)RNAP (SEQ ID NO: 6 and SEQ ID NO: 11, respectively). For (K11)RNAP, the RNAP portion A, herein referred to as (K11)RNAPN, comprises residues 1-199 of (K11)RNAP, and the RNAP portion B, herein referred to as (K11)RNAPC, comprises residues 200-906 of (K11)RNAP (SEQ ID NO: 7 and SEQ ID NO: 12, respectively). For (SP6)RNAP, the RNAP portion A, herein referred to as (SP6)RNAPN, comprises residues 1-148 of (SP6)RNAP, and RNAP portion B, herein referred to as (SP6)RNAPC, comprises residues 149-874 of (SP6)RNAP (SEQ ID NO: 8 and SEQ ID NO: 13, respectively).

By introducing mutations in DNA-recognition regions of RNAP portion Bs, a series of orthogonal RNAP portion Bs can be generated each recognizing only one of specific/unique promoters (promoter sequences are disclosed in SEQ ID NO: 44-SEQ ID NO: 51), and each configured to be associated with the same RNAP portion A. For example, one set of RNAP portion Bs comprises: T7-RNAP_{C}, (T7)CGG-RNAP_{C}, (T7)K1F-a-RNAP_{C}, (T7)K1F-b-RNAP_{C}, (T7)K1F-c-RNAP_{C}, (T7)CTGA-RNAP_{C}, (T7)T3-RNAP_{C}, and (T7)T3-R-RNAP_{C} (see SEQ ID NO: 10 and SEQ ID NO: 14-SEQ ID NO: 20, respectively). After proximity-driven assembly with the unmodified (T7)RNAPN (SEQ ID NO: 5) or the proximity-dependent (T7)RNAP_{N}* (see Pu J, et al., Nat Chem Biol. 2017, and SEQ ID NO: 9), these RNAP portion Bs enable orthogonal RNA transcription from the specific DNA promoters: (T7)RNAP_{C} assembled with (T7)RNAPN (or (T7)RNAP_{N}*) transcribes specifically from promoter PT7 (SEQ ID NO: 44); (T7)CGG-RNAP_{C} assembled with (T7)RNAPN (or (T7)RNAP_{N}*) transcribes specifically from promoter PCGG (SEQ ID NO: 45); (T7)K1F-a-RNAP_{C} assembled with (T7)RNAPN (or (T7)RNAP_{N}*) transcribes specifically from promoter PK1F (SEQ ID NO: 46); (T7)K1F-b-RNAP_{C} assembled with (T7)RNAPN (or (T7)RNAP_{N}*) transcribes specifically from promoter PK1F (SEQ ID NO: 46); (T7)K1F-c-RNAP_{C} assembled with (T7)RNAPN (or (T7)RNAP_{N}*) transcribes specifically from promoter PK1F (SEQ ID NO: 46); (T7)CTGA-RNAP_{C} assembled with (T7)RNAP_{N} (or (T7)RNAP_{N}*) transcribes specifically from promoter P_{CTGA} (SEQ ID NO: 47); (T7)T3-RNAP_{C} assembled with (T7)RNAP_{N} (or (T7)RNAP_{N}*) transcribes specifically from promoter PT3 (SEQ ID NO: 48); and (T7)T3-R-RNAP_{C} assembled with (T7)RNAPN (or (T7)RNAP_{N}*) transcribes specifically from promoter PT3 (SEQ ID NO: 48).

Furthermore, because of the homologous sequences and functions amongst (T7)RNAP, (T3)RNAP, (K11)RNAP, and (SP6)RNAP enzymes, the mutational variants experimentally validated in (T7)RNAP_{C} can be generated at homologous positions in (T3)RNAP_{C}, (K11)RNAP_{C}, and (SP6)RNAP_{C} for orthogonal DNA-promoter specific RNA transcription, thus expanding the repertoire of proximity-dependent split RNA polymerases for the polypeptide analysis applications described herein. Transferring amino acid identities of DNA-recognition sites from a reference protein to a structurally and functionally homologous protein at homologous positions will transfer the DNA-recognition function of the reference protein to the homologous protein.

Thus, (T3)RNAPC (SEQ ID NO: 11) assembled with (T3)RNAPN (SEQ ID NO: 6) transcribes specifically from promoter PT3 (SEQ ID NO: 48); (T3)CGG-RNAPC (SEQ ID NO: 21) assembled with (T3)RNAPN transcribes specifically from promoter P_{CGG} (SEQ ID NO: 45); (T3)K1F-a-RNAPC (SEQ ID NO: 22) assembled with (T3)RNAPN transcribes specifically from promoter P_{K1F} (SEQ ID NO: 46); and (T3)CTGA-RNAPC (SEQ ID NO: 25) assembled with (T3)RNAPN transcribes specifically from promoter P_{CTGA} (SEQ ID NO: 47). Similarly, (K11)RNAPC (SEQ ID NO: 12) assembled with (K11)RNAPN (SEQ ID NO: 7) transcribes specifically from promoter P_{K11} (SEQ ID NO: 50); (K11)CGG-RNAPC (SEQ ID NO: 27) assembled with (K11)RNAPN transcribes specifically from promoter P_{CGG} (SEQ ID NO: 45); (K11)K1F-a-RNAPC (SEQ ID NO: 28) assembled with (K11)RNAPN transcribes specifically from promoter P_{K1F} (SEQ ID NO: 46); (K11)CTGA-RNAPC (SEQ ID NO: 31) assembled with (K11)RNAPN transcribes specifically from promoter P_{CTGA} (SEQ ID NO: 47); (K11)T3-RNAPC (SEQ ID NO: 32) assembled with (K11)RNAPN transcribes specifically from promoter P_{T3} (SEQ ID NO: 48). Additionally, (SP6)RNAPC (SEQ ID NO: 13) assembled with (SP6)RNAPN (SEQ ID NO: 8) transcribes specifically from promoter P_{SP6} (SEQ ID NO: 49); (SP6)CGG-RNAPC (SEQ ID NO: 34) assembled with (SP6)RNAPN transcribes specifically from promoter P_{CGG} (SEQ ID NO: 45); (SP6)K1F-a-RNAPC (SEQ ID NO: 35) assembled with (SP6)RNAPN transcribes specifically from promoter P_{K1F} (SEQ ID NO: 46); (SP6)CTGA-RNAPC (SEQ ID NO: 38) assembled with (SP6)RNAPN transcribes specifically from promoter P_{CTGA} (SEQ ID NO: 47); (SP6)T3-RNAPC (SEQ ID NO: 39) assembled with (SP6)RNAP_{N} transcribes specifically from promoter P_{T3} (SEQ ID NO: 48).

Pairwise sequence alignment of the RNAP portion As (SEQ ID NO: 5-SEQ ID NO: 9) from the Sequence Listing that can be utilized for the high-throughput polypeptide analysis methods indicates about 33% or higher sequence identity.

Pairwise sequence alignment of the RNAP portion Bs (SEQ ID NO: 10-SEQ ID NO: 43) from the Sequence Listing that can be utilized for the high-throughput polypeptide analysis methods indicates about 32% or higher sequence identify.

Overall, considering availability of eight distinct promoters (see SEQ ID NO: 44-SEQ ID NO: 51) and the disclosed sequences (see SEQ ID NO: 5-SEQ ID NO: 43), up to eight functional orthogonal RNAP enzymes based on the same RNAPN variant and different RNAP_{C} variants can be utilized for the high-throughput polypeptide analysis methods disclosed herein.

### Example 4. Generating a Set of Functional RNAP Enzymes Comprising RNAP Portion Bs with Orthogonal Promoter Specificity—Approach III

As described in Meyer A J, et al., 2015, to increase engineered T7-related RNAP enzymes activity and specificity, a compartmentalized partnered replication (CPR) approach was utilized to select for T7 RNAP mutants capable of specifically recognizing six orthogonal promoters (disclosed in SEQ ID NO: 44-SEQ ID NO: 48, SEQ ID NO: 50, and SEQ ID NO: 51). The selected mutants display comparable activity to the wild-type enzyme (T7 RNAP) with limited cross-reactivity in both in vivo and in vitro transcription assays (Meyer A J, et al., 2015).

In order to generate specificity variants of T7 RNAP, a strain of E. coli was generated in which the production of Taq DNA polymerase (DNAP) was dependent on the use of a synthetic T7 promoter. Individual cells were compartmentalized in a water-in-oil emulsion with 5′-biotinylated primers for PCR amplification of the T7 RNAP specificity determining region. T7 RNAP variants capable of specifically recognizing the synthetic promoters will selectively produce Taq DNAP protein and be amplified by the Taq DNAP during PCR.

A library of T7 RNAP variants was generated in which a region encompassing amino acid residues 663-793 of SEQ ID NO: 1 was subjected to error-prone PCR (at a rate of two mutations per kb) and to PCR optimization. After six further rounds of selection (with error-prone PCR at rounds 8, 10, 11, and 13) 288 clones from the library were assayed for in vivo GFP expression from P_{CTGA} (SEQ ID NO: 47). Active clones (judged by fluorescence readings) were sequenced, and several mutations were found to occur frequently in the population (I681L, Q744K, H772R, E775V, and to a lesser extent V725A, T748S, and L749I). Different combinations of the selected mutations were added to the original library, which contained randomized variants at positions (R746, L747, N748, R756, L757, and Q758), which are most responsible for promoter recognition (Meyer A J, et al., 2015), and then assayed for activity in the P_{CTGA}-GFP strain. The resulting RNAP variants for each promoter were screened not only for their ability to drive GFP from their cognate promoters, but also for their cross-reactivities to related promoters (SEQ ID NO: 44-SEQ ID NO: 51).

The following list of orthogonal RNAP portion Bs have been identified and shown in the next paragraph, wherein after complementation with the RNAP portion A (SEQ ID NO: 5), the resulting engineered RNAP enzymes showed specific initiation of transcription from the corresponding promoters (Meyer A J, et al., 2015).

The functional RNAP enzyme consisting of the RNAP portion A (SEQ ID NO: 5) and the RNAP portion B (SEQ ID NO: 10) is 100% active on its cognate promoter (wildtype, SEQ ID NO: 44) and had at least a 75-fold selectivity. The functional RNAP enzyme consisting of the RNAP portion A (SEQ ID NO: 5) and the RNAP portion B (SEQ ID NO: 14; Q744K, L747V, N748H, L749I, R756E, L757M, H772R compared to SEQ ID NO: 10) was 31% active on its cognate promoter (SEQ ID NO: 45) and had at least a 34.3-fold selectivity. The functional RNAP enzyme consisting of the RNAP portion A (SEQ ID NO: 5) and the RNAP portion B (SEQ ID NO: 18; V725A, Q744K, L747I, N748S, L749I, R756T, Q758K, H772R, E775V compared to SEQ ID NO: 10) was 27% active on its cognate promoter (SEQ ID NO: 47) and had at least a 33.8-fold selectivity. The functional RNAP enzyme consisting of the RNAP portion A (SEQ ID NO: 5) and the RNAP portion B (SEQ ID NO: 20; T745K, N748D, L749M, M750I, H772R, E775V compared to SEQ ID NO: 10) was 40% active on its cognate promoter (SEQ ID NO: 48) and had at least a 46-fold selectivity. The functional RNAP enzyme consisting of the RNAP portion A (SEQ ID NO: 5) and the RNAP portion B (SEQ ID NO: 17; L749I, Q754S, R756N, I761V, H772R, Q786H compared to SEQ ID NO: 10) was 20% active on its cognate promoter (SEQ ID NO: 46) and had at least a 9.5-fold selectivity. The functional RNAP enzyme consisting of the RNAP portion A (SEQ ID NO: 5) and the RNAP portion B (SEQ ID NO: 43; L747I, N748D, L749C, M750V, F751I, Q754T, F755R, L757M, Q758A, P759L, D770N, H772R, E775V compared to SEQ ID NO: 10) was 15% active on its cognate promoter (SEQ ID NO: 51) and had at least a 9.8-fold selectivity.

The orthogonal RNAP fragments shown in the previous paragraph can be used in the disclosed methods, wherein each of the RNAP portion Bs is attached to a unique binding agent capable of binding to a portion of the macromolecule analyte. Binding between the RNAP portion A and each of the RNAP portion Bs produces six different functional RNAP enzymes configured to initiate transcription from the corresponding specific promoters comprising the following sequences: SEQ ID NO: 44-SEQ ID NO: 48 and SEQ ID NO: 51, which together comprise the plurality of promoters present on the recording tag.

### Example 5. Approaches to Engineer Novel Orthogonal RNAPC Fragments with DNA Promoter Specificity

Several approaches may be employed to generate a more robust system with a higher number of orthogonal RNAPC fragments that bind specifically to naturally occurring or engineered DNA promoters. RNAPC fragment variants may be generated experimentally using phage-assisted continuous evolution (PACE) (see Pu J, et al., Nat Chem Biol. 2017). Alternatively, computationally designed RNAPC fragments may be engineered from co-crystal structures of RNAP enzymes bound to DNA promoters from the Research Collaboratory for Structural Bioinformatics (RCSB) Protein Data Bank (PDB). The protein could be bisected (split) into two separate chains at the split points indicated above. Using PyRosetta macromolecular modeling and design software (S. Chaudhury, et al., 2010), novel RNAPC fragment variants and DNA promoter sequences may be computationally designed using Markov Chain Monte Carlo (MCMC) sampling of sidechain rotamers and nucleobase rotamers within a 12 Å distance (as measured by RNAPC residue C_{α} atom to DNA promoter pentose sugar C1′ atom distances) of residues at the interface of the RNAPC and the DNA promoter, respectively. Multiple iterations of MCMC rotamer sampling followed by global minimization of the co-complex backbone and sidechain conformations in a Rosetta energy function for flexible backbone design (Loshbaugh A L, Kortemme T. 2020) enables adequate sampling of sequence-structure space to generate RNAPC fragment libraries and DNA promoter libraries for experimental screening and selection. Further improved RNAPC fragment variants and DNA promoter sequences may be generated with the use of chemical principles by modeling the co-crystal structures of RNAP enzymes bound to DNA promoters and rationally designing (Gordon S R, et al. 2012) the amino acid residues and nucleobases at the interface between RNAPC and DNA promoters. Computationally designed RNAPC variants and DNA promoters can be synthesized for experimental screening using a luciferase-based transcription assay and selected for orthogonal RNAPC variants with DNA promoter specificity.

The aforementioned luciferase-based transcription assay of orthogonal C-terminal RNAP variants in E. coli, as described in Pu J, et al., J Am Chem Soc., 2017, enables selection of orthogonal RNAPC variants with DNA promoter specificity. Briefly, S1030 cells are transformed by electroporation with three plasmids: (i) an expression vector for an RNAPN fragment fused to the ZA protein, (ii) an expression vector for a computationally designed or experimentally mutated RNAPC fragment variant fused to the ZB protein to be tested, and (iii) a reporter vector that encodes a luciferase enzyme under control of a target DNA promoter sequence. The S1030 cell transformants are plated, single colonies grown to saturation overnight at 37° C., then cultures re-inoculated into 96-well deep-well plates in 0.6 mL final volumes of LB broth with antibiotics and 10 mM arabinose, and shaken at 37° C. for 3 hours. Subsequently, 150 μL of each culture is transferred to a 96-well black-walled, clear-bottom plate, and luminescence and OD600 is measured on a multimodal plate reader. Data may be analyzed by dividing the luminescence values by the background-corrected OD600 value, and then subtracting out the background luminescence from a well with cells expressing the reporter vector only. Novel RNAPC fragment variants and novel DNA promoters may then be analyzed for orthogonality. Finally, to quantify the proximity-dependence of RNAPN fragments and engineered RNAP_{C} fragments for transcription initiation, the same assay may be used by testing each RNAP_{C} fragment variant with its on-target DNA promoter, and comparing data from the RNAPN fused to ZA construct with a construct that is lacking the ZA fusion (see Pu J, et al., J Am Chem Soc. 2017). The engineered orthogonal proximity-dependent split RNA polymerases may be further assayed using an in vivo split GFP assay, and a dual reporter protein-protein interaction in vivo detection assay in S1030 cells (Pu J, et al., Nat Chem Biol. 2017).

### Example 6. Sequencing Produced RNA Transcripts

A variety of approaches can be used to collect identifying information from RNA transcripts produced in the disclosed polypeptide analysis methods.

In one embodiment, RNA transcripts can be sequenced directly using the Direct RNA Sequencing Kit (SQK-RNA002) by Oxford Nanopore Technologies according to the manufacturer's instructions. In this embodiment, sequencing RNA transcripts can be performed via a protocol in which RNA is prepared with a 3′ polyadenylation (poly-A) tail for RNA sequencing on an Oxford Nanopore sequencing device or similar nanopore-based sequencing device capable of reading and recording RNA sequences. In such a nucleic acid sequencing device, RNA molecules pass through a nanopore sensor, and the output read length reflects the lengths of the RNA molecules recovered in each binding cycle of the disclosed methods. In another embodiment, at least partial sequences of the RNA transcripts may be deciphered using the nanopore sequencing device. Several additional preliminary experimental steps may be required in preparing RNA transcripts for nanopore sequencing, which involve primer annealing, ligation, and attachment of a sequencing adaptor to directly or indirectly contact the nanopore protein prior to sequencing.

In another embodiment, instead of nanopore-based sequencing, Illumina sequencing technologies (or similar sequencing technologies) can be utilized to sequence produced RNA transcripts. RNA transcripts can be first reverse-transcribed into complementary DNA (cDNA) molecules using a reverse transcriptase enzyme. The cDNA may then be amplified by polymerase chain reaction (PCR) using Illumina adaptor primers in order to anneal the cDNA of interest to a sequencer flow cell.

In yet another embodiment, other sequencing technologies known in the art can be utilized to sequence produced RNA transcripts or corresponding cDNA molecules.

### Example 7. Exemplary Encoding of a Polypeptide Sequence by Proximity-Dependent Split RNA Polymerases

In this example, each macromolecule analyte from a plurality of different macromolecule analytes present in a biological sample is immobilized on a porous bead (solid support) by nucleic acid hybridization methods through an associated DNA recording tag as disclosed in Example 1 and in US 2022/0049246 A1. The recording tag associated with a macromolecule analyte is covalently linked through DNA ligation to a capture DNA hairpin attached to the bead. The recording tag associated with a polypeptide comprises a concatenation of different RNA polymerase (RNAP) promoters located sequentially, followed by the polypeptide-specific barcode, an optional short spacer region to reach the high-quality portion of Nanopore/Illumina sequencing reads, a poly-A tail to enable use of existing Oxford Nanopore Direct cDNA Sequencing kit, and a T7 terminator sequence to terminate RNA transcription (see FIG. 1A). The nucleic acid spacer region sequence, the poly-A tail sequence, and the T7 terminator sequence are optional elements which may not be present in other embodiments.

The polypeptide is covalently conjugated to the DNA hairpin on the bead, and the RNAP portion A (RNAPN) is non-covalently attached to the DNA hairpin on the bead but proximal to the promoter sequences (FIG. 1A). This recording tag architecture relies on only one polypeptide barcode, allowing poly-N sequences to be synthesized such that one polypeptide barcode corresponds to a single macromolecule analyte (i.e., polypeptide) attached to the bead, enabling bioinformatic analysis with a single randomized poly-N nucleic acid barcode per polypeptide.

To enable sequencing of the immobilized polypeptide, NTAA binding agents (engineered metalloproteins derived from human carbonic anhydrase II as disclosed in US 2022/0283175 A1) are covalently attached to orthogonal, promoter-specific C-terminal fragments of RNAP (RNAPC; also named RNAP portion Bs herein). Development of a set of orthogonal RNAP portion Bs are disclosed in Examples 2-4 above. Each NTAA binding agent is fused to a different orthogonal, promoter-specific RNAPC (FIG. 1A).

M64 N-terminal modification (NTM) that coordinates a zinc ion (Zn(II)) was installed on target polypeptides to provide more binding surface and achieve better specificity during binding agent engineering. M64 structure and installation method are shown below. In other embodiments, different N-terminal modifications can be utilized, such as those disclosed in US 2022/0283175 A1. In yet another embodiment, no N-terminal modifications are necessary, and binding agents used are configured to recognize unmodified NTAAs (or other epitopes) of immobilized polypeptide analytes.

Exemplary method of installing M64 onto the N-terminal amino acid of a polypeptide, shown as NTAA-PP:

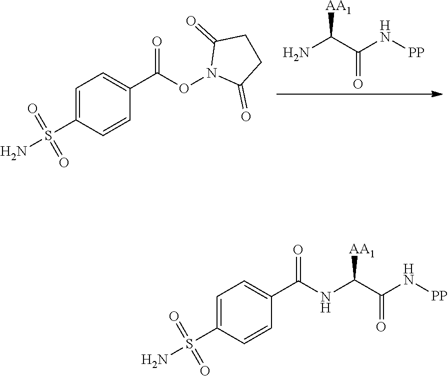

Polypeptides, in solution or on a solid support, were dissolved in 25 μL of 0.4 M MOPS buffer, pH=7.6 and 25 μL of acetonitrile (ACN). Separately, the active ester reagent was prepared from M64 and dissolved in 25 μL DMA and 25 μL ACN to a concentration of 0.05 M stock solution. Then, 50 μL of the active ester stock solution was added to the polypeptide-ACN:MOPS solution and incubated at 65° C. for 60 minutes. Upon completion, the polypeptides were functionalized with the respective modification as shown in the above schemes.

Alternatively, a surfactant-aqueous coupled system can be employed to install NTM (M64) onto the N-terminal amino acid of polypeptides. Using a 10 mM solution of 5% DMSO in 2% TGPS-750-M in water containing 1% 2,6-lutidine, the polypeptides are modified to completion in 20 minutes at 40° C.

Specific binding agents were successfully selected against M64-modified D, F, E, T and hydrophobic NTAA residues as disclosed in US 2022/0283175 A1 (sequences of binding agents are set forth in SEQ ID NO: 55-SEQ ID NO: 59, respectively). Optionally, concentrations of binding agents are fixed at their on-target NTAA Kd, enabling uniform encoding rates per reaction site.

A mix of binding agent-RNAPC fusion proteins is prepared and aliquoted onto the beads containing immobilized polypeptide analytes associated with (i) an RNAP portion A (RNAPN; the sequence is set forth in SEQ ID NO: 5), and (ii) a recording tag comprising an analyte-specific barcode and a plurality of corresponding promoters cognate for the RNAP_{C} proteins used in the fusions with binding agents (FIG. 1A). When one of the binding agents binds to the M64-modified NTAA of the immobilized polypeptide analyte, the RNAPN and RNAPC fragments are brought into close proximity resulting in formation of a functional RNAP enzyme and promoter-specific transcription from the recording tag on the bead (FIG. 1B). The exemplary reaction conditions for transcription are as follows: 40 mM Tris-HCl (pH 7.9), 10 mM NaCl, 6 mM MgCl₂, 10 mM DTT, 2 mM spermidine, 0.05% Tween®-20, 0.5 mM each of rATP, rGTP, rCTP and rUTP for 10 min at 37° C.

The resulting RNA transcripts are released into solution. The process of binding and DNA transcription proceeds for a period of time allowing for at least several RNA transcripts to be produced, each RNA transcript corresponding to the binding of a specific binding agent to M64-modified NTAAs of polypeptide analytes immobilized on the beads. Because the promoters are joined contiguously, the length of the RNA transcript encodes the binding agent identity (FIG. 1B). In the next step, the generated RNA transcripts (and binding agents) are collected for further analysis and aliquoted into a separate compartment for reverse transcription polymerase chain reaction (RT-PCR). The polypeptide analytes on the beads are then subjected to the N-terminal cleavage (NTC) produced by the engineered Cleavase enzyme as disclosed in U.S. Ser. No. 11/427,814 B2, incorporated herein by reference. A 1 μM solution of a mixture of 7 engineered cleavase enzymes configured to cleave all M64-modified NTAA residues from immobilized polypeptide analytes (each of the engineered cleavase enzymes has a sequence having at least 30% sequence identity to SEQ ID NO: 60) is added to beads with immobilized polypeptide analytes in 0.05M MES (2-(N-morpholino)ethanesulfonic acid) with 0.1% Tween 20 at pH 6.4, and incubated for 30 min at 65° C. The progress of the cleavage event can be monitored by taking aliquots of the reaction and injecting on the LC-MS. Completion of the cleavage indicates the end of the first binding cycle, where identifying information regarding the NTAA residues of immobilized polypeptide analytes was encoded in structures of the produced RNA transcripts.

Alternatively, instead of performing an enzymatic cleavage, NTAA residues of immobilized polypeptide analytes can be removed by mild chemical cleavage reagents as disclosed in US 2022/0227889 A1, incorporated herein by reference.

After completion of the NTAA cleavage, the next binding cycle starts with N-terminal M64 functionalization (NTF) on newly formed NTAA residues of immobilized polypeptide analytes, as it was described above. To avoid any carry-over of RNA transcripts from the previous cycle, the functionalization can be optionally performed in the presence of RNase to degrade any remaining trace RNA transcripts produced in the previous cycle. Then, the steps of addition of binding agent-RNAPC fusion proteins followed by RNA transcript generation are repeated, optionally in the presence of RNase inhibitors (FIG. 1C). As described above, when one of the binding agents binds to the M64-modified new NTAA of the immobilized polypeptide analyte, the RNAPN and RNAPC fragments are brought into a close proximity resulting in formation of a functional RNAP enzyme and promoter-specific transcription from the recording tag on the bead (FIG. 1B). The steps of NTAA functionalization, binding, RNA transcript generation followed by collection, and, optionally, NTAA cleavage are repeated sequentially one or more times until most amino acid residues of immobilized polypeptide analytes are cleaved off, or until enough information regarding identities of NTAA residues are collected (partial identity for a desired number of amino acid residues were determined).

The RNA transcripts generated during each cycle are collected and prepared for next-generation sequencing in a separate compartment. Each pool of RNA transcripts from one cycle gets barcoded with a cycle-specific barcode (BC_{Cycle}) during RT-PCR to produce complementary DNA (cDNA) for next-generation sequencing with Oxford Nanopore Technologies (ONT) technology or Illumina technology. To be compatible with Illumina next-generation sequencing platforms, full-length cDNA may be produced; also, strategies for non-templated poly-C extension and strand switching by reverse transcriptase or ligation of a unique DNA fragment to act as a template for forward priming may be used. Alternatively, to be compatible with Oxford Nanopore next-generation sequencing platforms, full-length cDNA may be produced with the Direct cDNA Sequencing Kit (ONT). RNA transcripts may also be directly sequenced using the Direct RNA Sequencing Kit (ONT).

Each library of cycle-specific cDNA or RNA is combined in approximately equimolar ratios and loaded onto a single Nanopore flow cell or Illumina flow cell for sequencing, followed by bioinformatic analysis of the sequencing data. To obtain polypeptide sequences, all polypeptide barcodes from a single bead functional site (BC_{Peptide} X) for each cycle barcode (BC_{Cycle} Y) is bioinformatically analyzed to count the ratios of read lengths per cycle to estimate or predict NTAA probability. To obtain polypeptide analyte quantities, the ratio of the sum of polypeptide barcode BC_{Peptide} X counts for all cycles to the sum of all polypeptide barcode counts for all cycles is bioinformatically analyzed, which should be roughly proportional to the polypeptide analyte quantities. It is noteworthy that while absolute polypeptide analyte quantities are not obtained, the ratio of the analyzed polypeptide analyte to all other polypeptide analytes in the sample can be obtained.

### Example 8. Analysis of Macromolecules Other than Polypeptides by Proximity-Dependent Split RNA Polymerases

The approaches described in the previous Examples can be adopted for other types of macromolecules, such as lipid, carbohydrate or macrocycle. To perform encoding assay, such macromolecules need to be immobilized on a solid support (such as beads) and associated with (i) an RNA polymerase (RNAP) portion A, and (ii) a recording tag comprising a plurality of promoters. The encoding steps remain the same regardless of the type of the macromolecule immobilized. The association with the recording tag and the RNAP portion A can be direct (such as covalent attachment) or indirect (such as association through a solid support). In the latter case, the recording tag should co-localize or be in a close proximity with the macromolecule during the encoding assay. Binding agents can be chosen to comprise binding moieties that bind specifically to a component of the macromolecule. Each binding agent needs to be conjugated to the RNAP portion B. The binding cycle can be repeated multiple times using different binding agents interacting with the macromolecule, either separately, or in a mixture. Below, representative methods known in the art are disclosed that can be utilized for adaptation of the disclosed encoding assay for macromolecules of different types, such as a carbohydrate, a lipid or a macrocycle.

First, exemplary binding moieties of binding agents that can specifically bind to components of a carbohydrate, a lipid or a macrocycle are known. For example, lectins are carbohydrate-binding proteins that can selectively recognize glycan epitopes of free carbohydrates or glycoproteins, and can be utilized as specific binding moieties for macromolecules that contain carbohydrates. Importantly, there are known lectins that recognize different components of carbohydrates, such as mannose-binding lectins, galactose/N-acetyl glucosamine-binding lectins, sialic acid/N-acetyl glucosamine-binding lectins, fucose-binding lectins (disclosed for example, in WO 2012/049285 A1). Also, lipid-binding proteins are well-known and can be utilized as binding moieties (see, for example, Bernlohr D A, et al., Intracellular lipid-binding proteins and their genes. Annu Rev Nutr. 1997; 17:277-303). Lipid-binding antibodies are commonly known and their antigen-binding fragments can be utilized as binding moieties for macromolecules that contain lipids (see, for example, Alving C R. Antibodies to lipids and liposomes: immunology and safety. J Liposome Res. 2006; 16(3):157-66). Furthermore, proteins that specifically bind to macrocycles are also known (see, for example, Villar E A, et al., How proteins bind macrocycles. Nat Chem Biol. 2014 September; 10(9):723-31; Hunter T M, et al., Protein recognition of macrocycles: binding of anti-HIV metallocyclams to lysozyme. Proc Natl Acad Sci USA. 2005 Feb. 15; 102(7):2288-92).

Second, an exemplary carbohydrate detection encoding assay can be performed as follows, utilizing methods known in the art.

Approach I. Reductive amination (based on Yang S J, Zhang H. Glycan analysis by reversible reaction to hydrazide beads and mass spectrometry. Anal Chem. 2012; 84(5):2232-2238).
(a) Generate an immobilized recording tag-attached carbohydrate conjugate. Oxidize carbohydrates with sodium periodate to generate an aldehyde. Conjugate amine terminated DNA recording tag and reduce the resulting imine using sodium cyanoborohydride to generate a carbohydrate-recording tag conjugate. Preferably, hydrazide, alkoxyamine, or similarly reactive DNA may be employed to generate more stable reaction products (e.g., hydrazones) that do not require reducing agents. Immobilize DNA-coupled carbohydrate to a solid support via the DNA recording tag through DNA hairpins having the RNAP portion A as described in Example 7.
(b) Generate lectin-RNAP portion B conjugates by utilizing SpyCatcher-concanavalin A (ConA) fusion. RNAP portion B fused to SpyTag is expressed separately and is reacted with SpyCatcher-concanavalin A to form the conjugates.
(c) Perform binding and encoding reaction as described in Example 7, thus analyzing whether the carbohydrate contains a component that binds to ConA.

Third, an exemplary lipid detection encoding assay can be performed as follows, utilizing methods known in the art.

Approach L Fatty acids (based on Hiroshi Miwa, High-performance liquid chromatographic determination of free fatty acids and esterified fatty acids in biological materials as their 2-nitrophenylhydrazides, Analytica Chimica Acta, Volume 465, Issues 1-2, 2002, Pages 237-255, ISSN 0003-2670).

Extract fatty acids from a biological source and activate carboxylic acid with EDC/CDI chemistry. Couple amine- or hydrazide-terminated DNA recording tag to generate a recording tag-attached lipid conjugate. Immobilize DNA-coupled lipid to a solid support via the DNA recording tag through DNA hairpins having the RNAP portion A as described in Example 7.

Approach IL Reactive lipids (based on X. Wei & H. Yin (2015) Covalent modification of DNA by a, (3-unsaturated aldehydes derived from lipid peroxidation: Recent progress and challenges, Free Radical Research, 49:7, 905-917).

Obtain a reactive lipid substrate such as malondialdehyde (MDA) or 4-hydroxynonenal (HNE); couple hydrazide-terminated DNA recording tag to reactive lipid species. Alternatively, couple amine-terminated DNA recording tag to aldehyde on reactive lipid and reduce resulting imine with sodium cyanoborohydride.

In the next step for both approaches, generate a binding agent-RNAP portion B conjugate by utilizing SpyCatcher-binding agent fusion as described earlier. Fatty acid-binding protein (FABP), other lipid binding proteins or lipid binding antibodies can be used as a binding agent. Finally, perform binding and encoding reaction as described in Example 7 of the present application, thus analyzing whether the lipid contains a component that binds to the binding agent.

Forth, an exemplary macrocycle (microcystin) detection encoding assay can be performed as follows, utilizing methods known in the art, based on McElhiney J, et al., Rapid isolation of a single-chain antibody against the cyanobacterial toxin microcystin-LR by phage display and its use in the immunoaffinity concentration of microcystins from water. Appl Environ Microbiol. 2002 November; 68(11):5288-95.

Generate DNA recording tag-coupled microcystin by reacting dehydroalanine of microcystin with 2-mercaptoethylamine to generate a primary amine, followed by coupling DNA recording tag to primary amine using an amine reactive DNA recording tag (e.g., NHS-DNA derivative).

Generate single chain antibody-SpyCatcher binding agent that recognizes microcystin. Single chain antibody production is described in McElhiney J, et al. 2002. Couple RNAP portion B to SpyTag, followed by reacting with SpyCatcher to generate the binding agent-RNAP portion B conjugate as described earlier.

Perform binding and encoding reaction as described in Example 7, thus analyzing whether the macromolecule contains microcystin.

The present disclosure is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES INCORPORATED BY REFERENCE FOR ALL PURPOSES

US 2019/0145982 A1, US 2020/0348308 A1, US 2020/0348307 A1, US 2021/0208150 A1, US 2022/0049246 A1, U.S. Ser. No. 11/427,814, US 2022/0227889 A1, US 2022/0283175 A1, US 2020/0332371 A1, US 2015/0368625 A1, US 2020/0199599 A1.
Pu J, et al., RNA Polymerase Tags To Monitor Multidimensional Protein-Protein Interactions Reveal Pharmacological Engagement of Bcl-2 Proteins. J Am Chem Soc. 2017; 139(34):11964-11972.
Pu J, et al., Evolution of a split RNA polymerase as a versatile biosensor platform. Nat Chem Biol. 2017 April; 13(4):432-438.
Temme K, et al., Modular control of multiple pathways using engineered orthogonal T7 polymerases. Nucleic Acids Res. 2012 Sep. 1; 40(17):8773-81;
Meyer A J, et al., Directed Evolution of a Panel of Orthogonal T7 RNA Polymerase Variants for in Vivo or in Vitro Synthetic Circuitry. ACS Synth Biol. 2015 Oct. 16; 4(10):1070-6.
Chelliserrykattil, J., Cai, G. and Ellington, A. D. (2001) A combined in vitro/in vivo selection for polymerases with novel promoter specificities. BMC Biotechnol., 1, 13.
Lee J H, Daugharthy E R, Scheiman J, Kalhor R, Yang J L, Ferrante T C, Terry R, Jeanty S S, Li C, Amamoto R, Peters D T, Turczyk B M, Marblestone A H, Inverso S A, Bernard A, Mali P, Rios X, Aach J, Church G M. Highly multiplexed subcellular RNA sequencing in situ. Science. 2014 Mar. 21; 343(6177):1360-3.
Segall-Shapiro T H, Meyer A J, Ellington A D, Sontag E D, Voigt C A. A ‘resource allocator’ for transcription based on a highly fragmented T7 RNA polymerase. Mol Syst Biol. 2014 Jul. 30; 10(7):742.
S. Chaudhury, S. Lyskov & J. J. Gray, “PyRosetta: a script-based interface for implementing molecular modeling algorithms using Rosetta,” Bioinformatics, 26(5), 689-691 (2010).
Gordon S R, Stanley E J, Wolf S, et al. Computational design of an a-gliadin peptidase. J Am Chem Soc. 2012; 134(50):20513-20520.
Loshbaugh A L, Kortemme T. Comparison of Rosetta flexible-backbone computational protein design methods on binding interactions. Proteins. 2020 January; 88(1):206-226. Epub 2019 Aug. 10. PMID: 31344278.
Brunette T J, Bick M J, Hansen J M, Chow C M, Kollman J M, Baker D. Modular repeat protein sculpting using rigid helical junctions. Proc Natl Acad Sci USA. 2020; 117(16):8870-8875.
Boyken S E, Chen Z, Groves B, Langan R A, Oberdorfer G, Ford A, Gilmore J M, Xu C, DiMaio F, Pereira JI-1, Sankaran B, Seelig G, Zwart P H, Baker D. De novo design of protein homo-oligomers with modular hydrogen-bond network-mediated specificity. Science. 2016 May 6; 352(6286):680-7.
Boyken S E, Benhaim M A, Busch F, et al. De novo design of tunable, pH-driven conformational changes. Science. 2019; 364(6441):658-664.

1. A method for analyzing a macromolecule analyte, comprising:
a) contacting the macromolecule analyte with a plurality of binding agents,
wherein the macromolecule analyte is attached to a solid support and associated with (i) an RNA polymerase (RNAP) portion A, and (ii) a recording tag comprising a plurality of promoters,
wherein each binding agent comprises (i) an RNAP portion B, and (ii) a binding moiety, and wherein upon binding between a first motif of the macromolecule analyte and the binding moiety of a first binding agent of the plurality of binding agents, the RNAP portion A associated with the macromolecule analyte and the RNAP portion B of the first binding agent are brought into proximity to form a first functional RNAP, which initiates transcription from a corresponding first promoter of the plurality of promoters to generate a first RNA transcript;
b) collecting the first RNA transcript, and removing the first functional RNAP enzyme or a portion thereof from the recording tag associated with the macromolecule analyte;
c) contacting the macromolecule analyte with the plurality of binding agents,
wherein upon binding between a second motif of the macromolecule analyte and the binding moiety of a second binding agent of the plurality of binding agents, the RNAP portion A associated with the macromolecule analyte and the RNAP portion B of the second binding agent are brought into proximity to form a second functional RNAP, which initiates transcription from a corresponding second promoter of the plurality of promoters to generate a second RNA transcript; and
d) analyzing the first and second RNA transcripts, thereby analyzing the first and second motifs of the macromolecule analyte.

2. The method of claim 1, wherein the plurality of promoters comprises at least four different promoters, and wherein the recording tag further comprises an analyte-specific barcode.

3. The method of claim 2, wherein at least four binding agents from the plurality of binding agents have different RNAP portion Bs, and at least four different functional RNAP enzymes are formed via interactions between the RNAP portion A and each of the four different RNAP portion Bs joined to the at least four binding agents, wherein each of the at least four different functional RNAP enzymes is specific to a different promoter of the plurality of promoters.

4. The method of claim 1, wherein 500 or more different macromolecule analytes are analyzed simultaneously, and each recording tag associated with one of the 500 or more different macromolecule analytes comprises a different analyte-specific barcode.

5. The method of claim 1, wherein the RNAP portion A associated with the macromolecule analyte is releasably or reversibly attached to the solid support, and/or
wherein the RNAP portion A associated with the macromolecule analyte is releasably or reversibly attached to the recording tag associated with the macromolecule analyte.

6. The method of claim 1, further comprising attaching the macromolecule analyte to the recording tag and attaching the macromolecule analyte and/or the recording tag to the solid support before performing step (a).

7. The method of claim 1, wherein the recording tag associated with the macromolecule analyte consists of a single DNA molecule which comprises the plurality of promoters,
optionally wherein the single DNA molecule comprises one or more spacers between adjacent promoters, and
optionally wherein the recording tag associated with the macromolecule analyte comprises a plurality of hairpin structures, and each hairpin structure comprises a stem that comprises one or more of the plurality of promoters.

8. The method of claim 1, wherein the recording tag associated with the macromolecule analyte comprises a plurality of DNA molecules attached to the solid support, and wherein each DNA molecule of the plurality of DNA molecules comprises a different promoter of the plurality of promoters.

9. The method of claim 1, which is a cell-free method.

10. The method of claim 1, wherein the RNAP portion A comprises an amino acid sequence having at least 30% sequence identity to any one of the amino acid sequences set forth in SEQ ID NO: 5 to SEQ ID NO: 9, and the RNAP portion B comprises an amino acid sequence having at least 30% sequence identity to any one of the amino acid sequences set forth in SEQ ID NO: 10 to SEQ ID NO: 43.

11. The method of claim 1, wherein the RNAP portion A and each of the RNAP portion Bs of the plurality of binding agents have no RNA polymerase activity by itself.

12. The method of claim 1, wherein the RNAP portion A does not bind to a promoter of the plurality of promoters; and wherein each of the RNAP portion Bs of the plurality of binding agents is specific for a promoter of the plurality of promoters, but does not initiate transcription without binding to the RNAP portion A.

13. The method of claim 1, wherein the interaction between the RNAP portion A and the RNAP portion B to form the first or second functional RNAP enzyme is increased relative to the interaction between the RNAP portion A and the RNAP portion B without joining to the first or second binding agent, and wherein binding between the binding agent and the macromolecule analyte promotes and/or stabilizes the association between the RNAP portion A and the RNAP portion B.

14. The method of claim 1, wherein the first functional RNAP together with the first promoter and the second functional RNAP together with the second promoter form orthogonal pairs.

15. The method of claim 1, wherein the macromolecule analyte comprises a polypeptide analyte.

16. The method of claim 15, wherein at step (d), at least a portion of an amino acid sequence of the polypeptide analyte is identified.

17. The method of claim 15, wherein at least the first binding agent and the second binding agent are capable of binding to an N-terminal amino acid (NTAA) of the polypeptide analyte, or are capable of binding to an N-terminal amino acid (NTAA) of the polypeptide analyte functionalized with a chemical moiety.

18. The method of claim 17, further comprising the following step: (0) modifying the NTAA of the polypeptide analyte with the chemical moiety to produce a functionalized NTAA of the polypeptide analyte, wherein the first binding agent is capable of binding to the modified NTAA of the polypeptide analyte, and step (0) is performed before step (a).

19. The method of claim 17, further comprising the following step: (bc) removing a portion of the polypeptide analyte, wherein the removed portion of the polypeptide analyte comprises the NTAA, or the NTAA functionalized with the chemical moiety, thereby yielding a newly exposed NTAA of the polypeptide analyte, and wherein step (bc) is performed after step (b) and before step (c).

20. The method of claim 19, wherein the portion of the polypeptide analyte removed at step (bc) comprises the NTAA functionalized with the chemical moiety, and the NTAA functionalized with the chemical moiety is removed from the polypeptide analyte by a modified cleavase enzyme.

21. A system for analyzing a plurality of macromolecule analytes, comprising:
a) the plurality of macromolecule analytes attached to a solid support, wherein each macromolecule analyte from the plurality of macromolecule analytes is associated with (i) an RNA polymerase (RNAP) portion A, and (ii) a recording tag comprising a plurality of promoters comprising at least a first promoter and a second promoter; and
b) a plurality of binding agents capable of binding to a macromolecule analyte of the plurality of macromolecule analytes and comprising at least a first binding agent and a second binding agent, wherein each binding agent of the plurality of binding agents comprises (i) an RNAP portion B, and (ii) a binding moiety, wherein the RNAP portion B of the first binding agent is different from any other RNAP portion Bs of binding agents of the plurality of binding agents, and wherein upon binding between a first motif of the macromolecule analyte and the binding moiety of the first binding agent, the RNAP portion A associated with the macromolecule analyte and the RNAP portion B of the first binding agent are brought into proximity to form a first functional RNAP, which initiates transcription from the first promoter to generate a first RNA transcript; and wherein
upon binding between a second motif of the macromolecule analyte and the binding moiety of the second binding agent, the RNAP portion A associated with the macromolecule analyte and the RNAP portion B of the second binding agent are brought into proximity to form a second functional RNAP, which initiates transcription from the second promoter to generate a second RNA transcript.

22. A method for analyzing a macromolecule analyte, comprising:
a) contacting the macromolecule analyte with a first binding agent,
wherein the macromolecule analyte is attached to a solid support and associated with (i) an RNA polymerase (RNAP) portion A, and (ii) a recording tag comprising a plurality of promoters,
wherein the first binding agent comprises (i) an RNAP portion B, and (ii) a binding moiety, and wherein upon binding between a first motif of the macromolecule analyte and the binding moiety of the first binding agent, the RNAP portion A associated with the macromolecule analyte and the RNAP portion B of the first binding agent are brought into proximity to form a first functional RNAP, which initiates transcription from a corresponding first promoter of the plurality of promoters to generate a first RNA transcript;
b) collecting the first RNA transcript;
c) removing the first binding agent from the macromolecule analyte, thereby removing the RNAP portion B from the recording tag and disrupting the first functional RNAP;
d) contacting the macromolecule analyte with a second binding agent,
wherein the second binding agent comprises (i) an RNAP portion B, and (ii) a binding moiety, and wherein upon binding between a second motif of the macromolecule analyte and the binding moiety of the second binding agent, the RNAP portion A associated with the macromolecule analyte and the RNAP portion B of the second binding agent are brought into proximity to form a second functional RNAP, which initiates transcription from a corresponding second promoter of the plurality of promoters to generate a second RNA transcript;
e) collecting the second RNA transcript; and
f) analyzing the first and second RNA transcripts, thereby analyzing the first and second motifs of the macromolecule analyte.


SEQUENCE LISTING (60 sequences)

SEQ ID NO: 1 - protein, 883 aa, Bacteriophage T7
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 2 - protein, 884 aa, Bacteriophage T3
MNIIENIEKNDFSEIELAAIPFNTLADHYGSALAKEQLALEHESYELGERRFLKMLERQAKAGEIADNAAAKPLLATLLPKLTTRIVEWLEEYASKKGRKPSAYAPLQLLKPEASAFITLKVILASLTSTNMTTIQAAAGMLGKAIEDEARFGRIRDLEAKHFKKHVEEQLNKRHGQVYKKAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVAITGGGYWANGRRPLALVRTHSKKGLMRYEDVYMPEVYKAVNLAQNTAWKINKKVLAVVNEIVNWKNCPVADIPSLERQELPPKPDDIDTNEAALKEWKKAAAGIYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVTHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLQKRLDMIFLGQFRLQPTINTLKDSGIDAHKQESGIAPNFVHSQDGSHLRMTVVYAHEKYGIESFALIHDSFGTIPADAGKLFKAVRETMVITYENNDVLADFYSQFADQLHETQLDKMPPLPKKGNLNLQDILKSDFAFA

SEQ ID NO: 3 - protein, 906 aa, Bacteriophage K11
MNALNIGRNDFSEIELAAIPYNILSEHYGDQAAREQLALEHEAYELGRQRFLKMLERQVKAGEFADNAAAKPLVLTLHPQLTKRIDDWKEEQANARGKKPRAYYPIKHGVASELAVSMGAEVLKEKRGVSSEAIALLTIKVVLGNAHRPLKGHNPAVSSQLGKALEDEARFGRIREQEAAYFKKNVADQLDKRVGHVYKKAFMQVVEADMISKGMLGGDNWASWKTDEQMHVGTKLLELLIEGTGLVEMTKNKMADGSDDVTSMQMVQLAPAFVELLSKRAGALAGISPMHQPCVVPPKPWVETVGGGYWSVGRRPLALVRTHSKKALRRYADVHMPEVYKAVNLAQNTPWKVNKKVLAVVNEIVNWKHCPVGDVPAIEREELPPRPDDIDTNEVARKAWRKEAAAVYRKDKARQSRRCRCEFMVAQANKFANHKAIWFPYNMDWRGRVYAVSMFNPQGNDMTKGSLTLAKGKPIGLDGFYWLKIHGANCAGVDKVPFPERIKFIEENEGNILASAADPLNNTWWTQQDSPFCFLAFCFEYAGVKHHGLNYNCSLPLAFDGSCSGIQHFSAMLRDSIGGRAVNLLPSDTVQDIYKIVADKVNEVLHQHAVNGSQTVVEQIADKETGEFHEKVTLGESVLAAQWLQYGVTRKVTKRSVMTLAYGSKESLVRQQVLEDTIQPAIDNGEGLMFTHPNQAAGYMAKLIWDAVTVTVVAAVEAMNWLKSAAKLLAAEVKDKKTKEVLRKRCAIHWVTPDGFPVWQEYRKQNQARLKLVFLGQANVKMTYNTGKDSEIDAHKQESGIAPNFVHSQDGSHLRMTVVHANEVYGIDSFALIHDSSGTIPADAGNLFKAVRETMVKTYEDNDVIADFYDQFADQLHESQLDKMPAVPAKGDLNLRDILESDFAFA

SEQ ID NO: 4 - protein, 874 aa, Bacteriophage SP6
MQDLHAIQLQLEEEMFNGGIRRFEADQQRQIAAGSESDTAWNRRLLSELIAPMAEGIQAYKEEYEGKKGRAPRALAFLQCVENEVAAYITMKVVMDMLNTDATLQAIAMSVAERIEDQVRFSKLEGHAAKYFEKVKKSLKASRTKSYRHAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMLRVRTCLMGDIKMSLQVETDIVDEAAMMGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 5 - protein, 179 aa, synthetic construct
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYK

SEQ ID NO: 6 - protein, 180 aa, synthetic construct
MNIIENIEKNDFSEIELAAIPFNTLADHYGSALAKEQLALEHESYELGERRFLKMLERQAKAGEIADNAAAKPLLATLLPKLTTRIVEWLEEYASKKGRKPSAYAPLQLLKPEASAFITLKVILASLTSTNMTTIQAAAGMLGKAIEDEARFGRIRDLEAKHFKKHVEEQLNKRHGQVYK

SEQ ID NO: 7 - protein, 199 aa, synthetic construct
MNALNIGRNDFSEIELAAIPYNILSEHYGDQAAREQLALEHEAYELGRQRFLKMLERQVKAGEFADNAAAKPLVLTLHPQLTKRIDDWKEEQANARGKKPRAYYPIKHGVASELAVSMGAEVLKEKRGVSSEAIALLTIKVVLGNAHRPLKGHNPAVSSQLGKALEDEARFGRIREQEAAYFKKNVADQLDKRVGHVYK

SEQ ID NO: 8 - protein, 148 aa, synthetic construct
MQDLHAIQLQLEEEMFNGGIRRFEADQQRQIAAGSESDTAWNRRLLSELIAPMAEGIQAYKEEYEGKKGRAPRALAFLQCVENEVAAYITMKVVMDMLNTDATLQAIAMSVAERIEDQVRFSKLEGHAAKYFEKVKKSLKASRTKSYR

SEQ ID NO: 9 - protein, 179 aa, synthetic construct
MNTINIAKNDFSDIELAAIPFNTLADHYGERSARGQLALEHESYEMGEARFRKMFERQLKAGEVADNAAAKPLITTLLPKMIARINDWFEEVKAKRGRRPTAFQFLKEIKPEAVAYITIKTSLACLTSADNTTVQAVASAIGRTIEDEARFGRIRDLEAKHFKKNVEEQLNKRVGHVYK

SEQ ID NO: 10 - protein, 704 aa, synthetic construct
KAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 11 - protein, 704 aa, synthetic construct
KAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVAITGGGYWANGRRPLALVRTHSKKGLMRYEDVYMPEVYKAVNLAQNTAWKINKKVLAVVNEIVNWKNCPVADIPSLERQELPPKPDDIDTNEAALKEWKKAAAGIYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVTHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLQKRLDMIFLGQFRLQPTINTLKDSGIDAHKQESGIAPNFVHSQDGSHLRMTVVYAHEKYGIESFALIHDSFGTIPADAGKLFKAVRETMVITYENNDVLADFYSQFADQLHETQLDKMPPLPKKGNLNLQDILKSDFAFA

SEQ ID NO: 12 - protein, 707 aa, synthetic construct
KAFMQVVEADMISKGMLGGDNWASWKTDEQMHVGTKLLELLIEGTGLVEMTKNKMADGSDDVTSMQMVQLAPAFVELLSKRAGALAGISPMHQPCVVPPKPWVETVGGGYWSVGRRPLALVRTHSKKALRRYADVHMPEVYKAVNLAQNTPWKVNKKVLAVVNEIVNWKHCPVGDVPAIEREELPPRPDDIDTNEVARKAWRKEAAAVYRKDKARQSRRCRCEFMVAQANKFANHKAIWFPYNMDWRGRVYAVSMFNPQGNDMTKGSLTLAKGKPIGLDGFYWLKIHGANCAGVDKVPFPERIKFIEENEGNILASAADPLNNTWWTQQDSPFCFLAFCFEYAGVKHHGLNYNCSLPLAFDGSCSGIQHFSAMLRDSIGGRAVNLLPSDTVQDIYKIVADKVNEVLHQHAVNGSQTVVEQIADKETGEFHEKVTLGESVLAAQWLQYGVTRKVTKRSVMTLAYGSKESLVRQQVLEDTIQPAIDNGEGLMFTHPNQAAGYMAKLIWDAVTVTVVAAVEAMNWLKSAAKLLAAEVKDKKTKEVLRKRCAIHWVTPDGFPVWQEYRKQNQARLKLVFLGQANVKMTYNTGKDSEIDAHKQESGIAPNFVHSQDGSHLRMTVVHANEVYGIDSFALIHDSSGTIPADAGNLFKAVRETMVKTYEDNDVIADFYDQFADQLHESQLDKMPAVPAKGDLNLRDILESDFAFA

SEQ ID NO: 13 - protein, 726 aa, synthetic construct
HAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMLRVRTCLMGDIKMSLQVETDIVDEAAMMGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 14 - protein, 704 aa, synthetic construct
KAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIKTRVHIMFLGQFEMQPTINTNKDSEIDARKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 15 - protein, 704 aa, synthetic construct
KAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLRFLGSFNLQPTVNTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 16 - protein, 704 aa, synthetic construct
KAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNIMFLGSFNLQPTVNTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 17 - protein, 704 aa, synthetic construct
KAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNIMFLGSFNLQPTVNTNKDSEIDARKQESGIAPNFVHSHDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 18 - protein, 704 aa, synthetic construct
KAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAAHWVTPDGFPVWQEYKKPIKTRISIMFLGQFTLKPTINTNKDSEIDARKQVSGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 19 - protein, 704 aa, synthetic construct
KAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQKRLDMIFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 20 - protein, 704 aa, synthetic construct
KAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQKRLDMIFLGQFRLQPTINTNKDSEIDARKQVSGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 21 - protein, 704 aa, synthetic construct
KAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVAITGGGYWANGRRPLALVRTHSKKGLMRYEDVYMPEVYKAVNLAQNTAWKINKKVLAVVNEIVNWKNCPVADIPSLERQELPPKPDDIDTNEAALKEWKKAAAGIYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVTHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLKKRVHIIFLGQFEMQPTINTLKDSGIDARKQESGIAPNFVHSQDGSHLRMTVVYAHEKYGIESFALIHDSFGTIPADAGKLFKAVRETMVITYENNDVLADFYSQFADQLHETQLDKMPPLPKKGNLNLQDILKSDFAFA

SEQ ID NO: 22 - protein, 704 aa, synthetic construct
KAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVAITGGGYWANGRRPLALVRTHSKKGLMRYEDVYMPEVYKAVNLAQNTAWKINKKVLAVVNEIVNWKNCPVADIPSLERQELPPKPDDIDTNEAALKEWKKAAAGIYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVTHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLQKRLDMRFLGSFNLQPTVNTLKDSGIDAHKQESGIAPNFVHSQDGSHLRMTVVYAHEKYGIESFALIHDSFGTIPADAGKLFKAVRETMVITYENNDVLADFYSQFADQLHETQLDKMPPLPKKGNLNLQDILKSDFAFA

SEQ ID NO: 23 - protein, 704 aa, synthetic construct
KAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVAITGGGYWANGRRPLALVRTHSKKGLMRYEDVYMPEVYKAVNLAQNTAWKINKKVLAVVNEIVNWKNCPVADIPSLERQELPPKPDDIDTNEAALKEWKKAAAGIYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVTHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLQKRLDIIFLGSFNLQPTVNTLKDSGIDAHKQESGIAPNFVHSQDGSHLRMTVVYAHEKYGIESFALIHDSFGTIPADAGKLFKAVRETMVITYENNDVLADFYSQFADQLHETQLDKMPPLPKKGNLNLQDILKSDFAFA

SEQ ID NO: 24 - protein, 704 aa, synthetic construct
KAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVAITGGGYWANGRRPLALVRTHSKKGLMRYEDVYMPEVYKAVNLAQNTAWKINKKVLAVVNEIVNWKNCPVADIPSLERQELPPKPDDIDTNEAALKEWKKAAAGIYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVTHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLQKRLDIIFLGSFNLQPTVNTLKDSGIDARKQESGIAPNFVHSHDGSHLRMTVVYAHEKYGIESFALIHDSFGTIPADAGKLFKAVRETMVITYENNDVLADFYSQFADQLHETQLDKMPPLPKKGNLNLQDILKSDFAFA

SEQ ID NO: 25 - protein, 704 aa, synthetic construct
KAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVAITGGGYWANGRRPLALVRTHSKKGLMRYEDVYMPEVYKAVNLAQNTAWKINKKVLAVVNEIVNWKNCPVADIPSLERQELPPKPDDIDTNEAALKEWKKAAAGIYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVTHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAAHWTTPDGFPVWQEYRKPLKKRISIIFLGQFTLKPTINTLKDSGIDARKQVSGIAPNFVHSQDGSHLRMTVVYAHEKYGIESFALIHDSFGTIPADAGKLFKAVRETMVITYENNDVLADFYSQFADQLHETQLDKMPPLPKKGNLNLQDILKSDFAFA

SEQ ID NO: 26 - protein, 704 aa, synthetic construct
KAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVAITGGGYWANGRRPLALVRTHSKKGLMRYEDVYMPEVYKAVNLAQNTAWKINKKVLAVVNEIVNWKNCPVADIPSLERQELPPKPDDIDTNEAALKEWKKAAAGIYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVTHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLQKRLDMIFLGQFRLQPTINTLKDSGIDARKQVSGIAPNFVHSQDGSHLRMTVVYAHEKYGIESFALIHDSFGTIPADAGKLFKAVRETMVITYENNDVLADFYSQFADQLHETQLDKMPPLPKKGNLNLQDILKSDFAFA

SEQ ID NO: 27 - protein, 707 aa, synthetic construct
KAFMQVVEADMISKGMLGGDNWASWKTDEQMHVGTKLLELLIEGTGLVEMTKNKMADGSDDVTSMQMVQLAPAFVELLSKRAGALAGISPMHQPCVVPPKPWVETVGGGYWSVGRRPLALVRTHSKKALRRYADVHMPEVYKAVNLAQNTPWKVNKKVLAVVNEIVNWKHCPVGDVPAIEREELPPRPDDIDTNEVARKAWRKEAAAVYRKDKARQSRRCRCEFMVAQANKFANHKAIWFPYNMDWRGRVYAVSMFNPQGNDMTKGSLTLAKGKPIGLDGFYWLKIHGANCAGVDKVPFPERIKFIEENEGNILASAADPLNNTWWTQQDSPFCFLAFCFEYAGVKHHGLNYNCSLPLAFDGSCSGIQHFSAMLRDSIGGRAVNLLPSDTVQDIYKIVADKVNEVLHQHAVNGSQTVVEQIADKETGEFHEKVTLGESVLAAQWLQYGVTRKVTKRSVMTLAYGSKESLVRQQVLEDTIQPAIDNGEGLMFTHPNQAAGYMAKLIWDAVTVTVVAAVEAMNWLKSAAKLLAAEVKDKKTKEVLRKRCAIHWVTPDGFPVWQEYRKQNKARVHIVFLGQAEMKMTYNTGKDSEIDARKQESGIAPNFVHSQDGSHLRMTVVHANEVYGIDSFALIHDSSGTIPADAGNLFKAVRETMVKTYEDNDVIADFYDQFADQLHESQLDKMPAVPAKGDLNLRDILESDFAFA

SEQ ID NO: 28 - protein, 707 aa, synthetic construct
KAFMQVVEADMISKGMLGGDNWASWKTDEQMHVGTKLLELLIEGTGLVEMTKNKMADGSDDVTSMQMVQLAPAFVELLSKRAGALAGISPMHQPCVVPPKPWVETVGGGYWSVGRRPLALVRTHSKKALRRYADVHMPEVYKAVNLAQNTPWKVNKKVLAVVNEIVNWKHCPVGDVPAIEREELPPRPDDIDTNEVARKAWRKEAAAVYRKDKARQSRRCRCEFMVAQANKFANHKAIWFPYNMDWRGRVYAVSMFNPQGNDMTKGSLTLAKGKPIGLDGFYWLKIHGANCAGVDKVPFPERIKFIEENEGNILASAADPLNNTWWTQQDSPFCFLAFCFEYAGVKHHGLNYNCSLPLAFDGSCSGIQHFSAMLRDSIGGRAVNLLPSDTVQDIYKIVADKVNEVLHQHAVNGSQTVVEQIADKETGEFHEKVTLGESVLAAQWLQYGVTRKVTKRSVMTLAYGSKESLVRQQVLEDTIQPAIDNGEGLMFTHPNQAAGYMAKLIWDAVTVTVVAAVEAMNWLKSAAKLLAAEVKDKKTKEVLRKRCAIHWVTPDGFPVWQEYRKQNQARLKLRFLGSANVKMTVNTGKDSEIDAHKQESGIAPNFVHSQDGSHLRMTVVHANEVYGIDSFALIHDSSGTIPADAGNLFKAVRETMVKTYEDNDVIADFYDQFADQLHESQLDKMPAVPAKGDLNLRDILESDFAFA

SEQ ID NO: 29 - protein, 707 aa, synthetic construct
KAFMQVVEADMISKGMLGGDNWASWKTDEQMHVGTKLLELLIEGTGLVEMTKNKMADGSDDVTSMQMVQLAPAFVELLSKRAGALAGISPMHQPCVVPPKPWVETVGGGYWSVGRRPLALVRTHSKKALRRYADVHMPEVYKAVNLAQNTPWKVNKKVLAVVNEIVNWKHCPVGDVPAIEREELPPRPDDIDTNEVARKAWRKEAAAVYRKDKARQSRRCRCEFMVAQANKFANHKAIWFPYNMDWRGRVYAVSMFNPQGNDMTKGSLTLAKGKPIGLDGFYWLKIHGANCAGVDKVPFPERIKFIEENEGNILASAADPLNNTWWTQQDSPFCFLAFCFEYAGVKHHGLNYNCSLPLAFDGSCSGIQHFSAMLRDSIGGRAVNLLPSDTVQDIYKIVADKVNEVLHQHAVNGSQTVVEQIADKETGEFHEKVTLGESVLAAQWLQYGVTRKVTKRSVMTLAYGSKESLVRQQVLEDTIQPAIDNGEGLMFTHPNQAAGYMAKLIWDAVTVTVVAAVEAMNWLKSAAKLLAAEVKDKKTKEVLRKRCAIHWVTPDGFPVWQEYRKQNQARLKIVFLGSANVKMTVNTGKDSEIDAHKQESGIAPNFVHSQDGSHLRMTVVHANEVYGIDSFALIHDSSGTIPADAGNLFKAVRETMVKTYEDNDVIADFYDQFADQLHESQLDKMPAVPAKGDLNLRDILESDFAFA

SEQ ID NO: 30 - protein, 707 aa, synthetic construct
KAFMQVVEADMISKGMLGGDNWASWKTDEQMHVGTKLLELLIEGTGLVEMTKNKMADGSDDVTSMQMVQLAPAFVELLSKRAGALAGISPMHQPCVVPPKPWVETVGGGYWSVGRRPLALVRTHSKKALRRYADVHMPEVYKAVNLAQNTPWKVNKKVLAVVNEIVNWKHCPVGDVPAIEREELPPRPDDIDTNEVARKAWRKEAAAVYRKDKARQSRRCRCEFMVAQANKFANHKAIWFPYNMDWRGRVYAVSMFNPQGNDMTKGSLTLAKGKPIGLDGFYWLKIHGANCAGVDKVPFPERIKFIEENEGNILASAADPLNNTWWTQQDSPFCFLAFCFEYAGVKHHGLNYNCSLPLAFDGSCSGIQHFSAMLRDSIGGRAVNLLPSDTVQDIYKIVADKVNEVLHQHAVNGSQTVVEQIADKETGEFHEKVTLGESVLAAQWLQYGVTRKVTKRSVMTLAYGSKESLVRQQVLEDTIQPAIDNGEGLMFTHPNQAAGYMAKLIWDAVTVTVVAAVEAMNWLKSAAKLLAAEVKDKKTKEVLRKRCAIHWVTPDGFPVWQEYRKQNQARLKIVFLGSANVKMTVNTGKDSEIDARKQESGIAPNFVHSHDGSHLRMTVVHANEVYGIDSFALIHDSSGTIPADAGNLFKAVRETMVKTYEDNDVIADFYDQFADQLHESQLDKMPAVPAKGDLNLRDILESDFAFA

SEQ ID NO: 31 - protein, 707 aa, synthetic construct
KAFMQVVEADMISKGMLGGDNWASWKTDEQMHVGTKLLELLIEGTGLVEMTKNKMADGSDDVTSMQMVQLAPAFVELLSKRAGALAGISPMHQPCVVPPKPWVETVGGGYWSVGRRPLALVRTHSKKALRRYADVHMPEVYKAVNLAQNTPWKVNKKVLAVVNEIVNWKHCPVGDVPAIEREELPPRPDDIDTNEVARKAWRKEAAAVYRKDKARQSRRCRCEFMVAQANKFANHKAIWFPYNMDWRGRVYAVSMFNPQGNDMTKGSLTLAKGKPIGLDGFYWLKIHGANCAGVDKVPFPERIKFIEENEGNILASAADPLNNTWWTQQDSPFCFLAFCFEYAGVKHHGLNYNCSLPLAFDGSCSGIQHFSAMLRDSIGGRAVNLLPSDTVQDIYKIVADKVNEVLHQHAVNGSQTVVEQIADKETGEFHEKVTLGESVLAAQWLQYGVTRKVTKRSVMTLAYGSKESLVRQQVLEDTIQPAIDNGEGLMFTHPNQAAGYMAKLIWDAVTVTVVAAVEAMNWLKSAAKLLAAEVKDKKTKEVLRKRCAAHWVTPDGFPVWQEYRKQNKARISIVFLGQATVKMTYNTGKDSEIDARKQVSGIAPNFVHSQDGSHLRMTVVHANEVYGIDSFALIHDSSGTIPADAGNLFKAVRETMVKTYEDNDVIADFYDQFADQLHESQLDKMPAVPAKGDLNLRDILESDFAFA

SEQ ID NO: 32 - protein, 707 aa, synthetic construct
KAFMQVVEADMISKGMLGGDNWASWKTDEQMHVGTKLLELLIEGTGLVEMTKNKMADGSDDVTSMQMVQLAPAFVELLSKRAGALAGISPMHQPCVVPPKPWVETVGGGYWSVGRRPLALVRTHSKKALRRYADVHMPEVYKAVNLAQNTPWKVNKKVLAVVNEIVNWKHCPVGDVPAIEREELPPRPDDIDTNEVARKAWRKEAAAVYRKDKARQSRRCRCEFMVAQANKFANHKAIWFPYNMDWRGRVYAVSMFNPQGNDMTKGSLTLAKGKPIGLDGFYWLKIHGANCAGVDKVPFPERIKFIEENEGNILASAADPLNNTWWTQQDSPFCFLAFCFEYAGVKHHGLNYNCSLPLAFDGSCSGIQHFSAMLRDSIGGRAVNLLPSDTVQDIYKIVADKVNEVLHQHAVNGSQTVVEQIADKETGEFHEKVTLGESVLAAQWLQYGVTRKVTKRSVMTLAYGSKESLVRQQVLEDTIQPAIDNGEGLMFTHPNQAAGYMAKLIWDAVTVTVVAAVEAMNWLKSAAKLLAAEVKDKKTKEVLRKRCAIHWVTPDGFPVWQEYRKQNQKRLDMIFLGQANVKMTYNTGKDSEIDAHKQESGIAPNFVHSQDGSHLRMTVVHANEVYGIDSFALIHDSSGTIPADAGNLFKAVRETMVKTYEDNDVIADFYDQFADQLHESQLDKMPAVPAKGDLNLRDILESDFAFA

SEQ ID NO: 33 - protein, 707 aa, synthetic construct
KAFMQVVEADMISKGMLGGDNWASWKTDEQMHVGTKLLELLIEGTGLVEMTKNKMADGSDDVTSMQMVQLAPAFVELLSKRAGALAGISPMHQPCVVPPKPWVETVGGGYWSVGRRPLALVRTHSKKALRRYADVHMPEVYKAVNLAQNTPWKVNKKVLAVVNEIVNWKHCPVGDVPAIEREELPPRPDDIDTNEVARKAWRKEAAAVYRKDKARQSRRCRCEFMVAQANKFANHKAIWFPYNMDWRGRVYAVSMFNPQGNDMTKGSLTLAKGKPIGLDGFYWLKIHGANCAGVDKVPFPERIKFIEENEGNILASAADPLNNTWWTQQDSPFCFLAFCFEYAGVKHHGLNYNCSLPLAFDGSCSGIQHFSAMLRDSIGGRAVNLLPSDTVQDIYKIVADKVNEVLHQHAVNGSQTVVEQIADKETGEFHEKVTLGESVLAAQWLQYGVTRKVTKRSVMTLAYGSKESLVRQQVLEDTIQPAIDNGEGLMFTHPNQAAGYMAKLIWDAVTVTVVAAVEAMNWLKSAAKLLAAEVKDKKTKEVLRKRCAIHWVTPDGFPVWQEYRKQNQKRLDMIFLGQANVKMTYNTGKDSEIDARKQVSGIAPNFVHSQDGSHLRMTVVHANEVYGIDSFALIHDSSGTIPADAGNLFKAVRETMVKTYEDNDVIADFYDQFADQLHESQLDKMPAVPAKGDLNLRDILESDFAFA

SEQ ID NO: 34 - protein, 726 aa, synthetic construct
HAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEKLRVHICLMGDIEMSLQVETDIVDERAMMGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 35 - protein, 726 aa, synthetic construct
HAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMLRVRTRLMGSINMSLQVETDIVDEAAMMGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 36 - protein, 726 aa, synthetic construct
HAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMLRVRICLMGSINMSLQVETDIVDEAAMMGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 37 - protein, 726 aa, synthetic construct
HAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMLRVRICLMGSINMSLQVETDIVDERAMMGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 38 - protein, 726 aa, synthetic construct
HAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGAMYTLPTGFILEQKIMATEKLRISICLMGDITMKLQVETDIVDERAMVGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 39 - protein, 726 aa, synthetic construct
HAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMKRVDMILMGDIKMSLQVETDIVDEAAMMGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 40 - protein, 726 aa, synthetic construct
HAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMKRVDMILMGDIKMSLQVETDIVDERAMVGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 41 - protein, 704 aa, synthetic construct
KAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGSFNLQPTVNTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 42 - protein, 704 aa, synthetic construct
KAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRIDCVILGTHRMALTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 43 - protein, 704 aa, synthetic construct
KAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRIDCVILGTRRMALTINTNKDSEINARKQVSGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 44 - DNA, 17 bp, synthetic construct
  source 1..17: mol_type = other DNA
taatacgactcactata

SEQ ID NO: 45 - DNA, 17 bp, synthetic construct
  source 1..17: mol_type = other DNA
taataccggtcactata

SEQ ID NO: 46 - DNA, 17 bp, synthetic construct
  source 1..17: mol_type = other DNA
taataactatcactata

SEQ ID NO: 47 - DNA, 17 bp, synthetic construct
  source 1..17: mol_type = other DNA
taatacctgacactata

SEQ ID NO: 48 - DNA, 17 bp, synthetic construct
  source 1..17: mol_type = other DNA
aattaaccctcactaaa

SEQ ID NO: 49 - DNA, 17 bp, synthetic construct
  source 1..17: mol_type = other DNA
atttaggtgacactata

SEQ ID NO: 50 - DNA, 17 bp, synthetic construct
  source 1..17: mol_type = other DNA
aattagggcacactata

SEQ ID NO: 51 - DNA, 17 bp, synthetic construct
  source 1..17: mol_type = other DNA
taataacccacactata

SEQ ID NO: 52 - protein, 178 aa, Rattus norvegicus
QDSTQNLIPAPPLISVPLQPGFWTERFQGRWFVVGLAGNAVQKERQSRFTMYSTIYELQEDNSYNVTSILVRGQGCRYWIRTFVPSSRPGQFTLGNIHSYPQIQSYDVQVADTDYDQFAMVFFQKTSENKQYFKVTLYGRTKGLSDELKERFVSFAKSLGLKDNNIVFSVPTDQCIDN

SEQ ID NO: 53 - protein, 164 aa, Homo sapiens
QVSVQPNFQQDKFLGRWFSAGLASNSSWLREKKAALSMAKSVVAPATDGGLNLTSTFLRKNQCETRTMLLQPAGSLGSYSYRSPHFGSTYSVSVVETDYDQYALLYSQGSKGPGEDFRMATLYSRTQTPRAELKEKFTAFSKAQGFTEDTIVFLPQTDKCMTEQ

SEQ ID NO: 54 - protein, 256 aa, synthetic construct
HWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASF

SEQ ID NO: 55 - protein, 256 aa, synthetic construct
HWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQHPDGLAVLGIFLKVGSAKPGLQKVVDVLDSIKTKGKSADFTNFDPRGLLPESLDYWTYPGSRTTPPLLESVTWIVLKEPISVSSEQVLKFRKLNFNGEGEPEELMVDNWRPAQPLKNRQIKASF

SEQ ID NO: 56 - protein, 256 aa, synthetic construct
HWGYGKHNGPEHWHKDFPIAKGERQSPVDIDTHTAKYDPSLKPLSVSYDQATSLRILNNGHAFEVEFDDSQDKAVLKGGPLDGTYRLVQFHFHWGSLDGQGSEHTVDKKKYAAELHLVHWNTKYGDYGKAMQQPDGLAVLGIFLKVGSAKPGLQKVVDVLDSIKTKGKSADFTNFDPRGLLPESLDYWTYPGSGTVPPLLESVTWIVLKEPISVSSEQVLKFRKLNFNGEGEPEELMVDNWRPAQPLKNRQIKASF

SEQ ID NO: 57 - protein, 256 aa, synthetic construct
HWGYGKHNGPEHWHKDFPIAKGERQSPVDIDTHTAKYDPSLKPLSVSYDQATSLRILNNGHAFLVEFDDSQDKAVLKGGPLDGTYRLKQFHFHWGSLDGQGSEHTVDKKKYAAELHLVHWNTKYGDYGKALQQPDGIAVLGIFLKVGSAKPGLQKVVDVLDSIKTKGKSADFTNFDPRGLLPESLDYWTYPGSRTIPPLLESVTWIVLKEPISVSSEQVLKFRKLNFNGEGEPEELMVDNWRPAQPLKNRQIKASF

SEQ ID NO: 58 - protein, 256 aa, synthetic construct
HWGYGKHNGPEHWHKDFPIAKGERQSPVDIDTHTAKYDPSLKPLSVSYDQATSLRILNDGHAFRVEFDDSQDKAVLKGGPLDGTYRLKQFHFHWGSLDGQGSEHTVDKKKYAAELHLVHWNTKYGDFGKAGQQPDGIAILGIFLKVGSAKPGLQKVVDVLDSIKTKGKSADFTNFDPRGLLPESLDYWTYPGSETIPPLLESVTWIVLKEPISVSSEQVLKFRKLNFNGEGEPEELMVDNWRPAQPLKNRQIKASF

SEQ ID NO: 59 - protein, 257 aa, synthetic construct
DWGYDDKNGPEQWSKLYPIANGNNQSPVDIKTSETKHDTSLKPISVSYNPATAKEIINVGHSFHVNFEDNDNRSVLKGGPFSDSYRLFQFHFHWGSTNEHGSEHTVDGVKYSAELHVAHWNSAKYSSLAEAASKADGLAVIGVLMKVGEANPKLQKVLDALQAIKTKGKRAPFTNFDPSTLLPSSLDFWTYPGSLTHPPLYESVTWIICKESISVSSEQLAQFRSLLSNVEGDNAVPMQHNNRPTQPLKGRTVRASF

SEQ ID NO: 60 - protein, 700 aa, Thermomonas hydrothermalis
DEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDNWMWPRHTGDFSFYRAYVGKDGKPAPYSKDNVPYRPKHWLKIADTPLGEGDFVMVAGYPGRTDRYALVAEFENTQNWLYPAISKAYKDQIALVEAAAKDNPEIAVKYAAALAGWNNTSKNFDGQLEGFKRNDVLAIKRREEAAVLEWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLDRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKETGVEPFDNPKALLEAVAAKRYAGLADARLGTVPVNFLADLDITGGNSGSPVLDANGRLVGLAFDGTLESVASNWVFDPVLTRMISVDQRYMRWIMQEVMPAPQLLEELGVPPRQ